SEQUENCE LISTING (10 sequences)

SEQ ID NO: 1 - DNA, 20 bp, synthetic construct
  source 1..20: mol_type = other DNA
tctggagacgcaggaagaga

SEQ ID NO: 2 - DNA, 20 bp, synthetic construct
  source 1..20: mol_type = other DNA
ctgtctccacatgcccagtt

SEQ ID NO: 3 - DNA, 720 bp, synthetic construct
  source 1..720: mol_type = other DNA
atggtgtctaagggcgaggaattgtttacaggtgtggtgcccatcctggtggagcttgatggcgatgtaaatggacacaaattctccgttagtggggaaggcgaaggggatgccacctacggtaagcttacgctgaaattcatctgcaccactggtaaactccccgtgccatggccaaccctggtcacgacccttacttatggggtgcagtgtttttcaaggtaccccgaccatatgaaacaacatgatttcttcaagtccgccatgccggaggggtacgtccaggagagaacaatctttttcaaagatgacgggaactacaagactcgcgcagaagtcaagtttgagggagacactctcgtaaaccgaattgaactgaaaggaattgactttaaggaagacggtaatatactgggccacaagctggagtataattataacagccataatgtgtatatcatggcagacaagcaaaagaacggcattaaagtgaacttcaagatccgtcacaatatcgaggatggcagcgtccagctggctgaccactaccagcagaacacacctattggagacggcccagttttactacctgacaaccactatctcagtacacagagcgccctctctaaggaccctaatgaaaagcgggatcatatggttttactggagtttgtcacagctgcgggaataaccttgggcatggatgagttgtacaaatga

SEQ ID NO: 4 - DNA, 1653 bp, synthetic construct
  source 1..1653: mol_type = other DNA
atggaagacgccaagaacattaagaagggccctgcacccttctacccactggaagacggtactgcaggggagcagctgcacaaggccatgaagcggtatgccctcgttcctggcactatcgccttcacagatgcccacatcgaagtagatatcacctatgctgagtactttgagatgagtgtgagactggcagaggcaatgaaacgttatggactgaacaccaaccatagaatcgtagtgtgctctgagaacagcttgcagttcttcatgcctgtcttaggagcactgttcataggcgtcgccgtggcaccagccaatgacatttacaatgagagggagctcctgaatagtatgggcataagccagccaacagtggttttcgtttccaagaaagggcttcaaaaaatcctgaatgtgcaaaagaagctccctatcatccagaagatcattataatggactcaaagactgattaccagggcttccagtccatgtatacatttgtcaccagccaccttccaccaggcttcaatgaatatgatttcgtgcctgagtcatttgacagggacaaaacaattgcactcattatgaattcttccgggtcaacaggtttgcccaagggggtggcgctaccacacagaacggcgtgtgtgcgctttagccatgctcgagaccccatcttcgggaatcagatcattcccgacactgccatcttgtctgtcgtcccttttcaccatggtttcggtatgttcaccacgttgggctacctgatctgtggtttccgggtagtactgatgtacaggtttgaagaagagctcttcctgcggagcctacaggactacaagatccagagcgcactgctggtgcccacccttttttcgttctttgccaaatccaccctgattgataaatatgacctatccaaccttcatgagatagcatctggaggtgctcctctgagtaaagaagtcggagaagctgtagccaagaggttccacctgccaggcattcgccaaggatatggcctgacagagactacaagtgccattttaataactccagagggagatgacaagcctggggctgtgggcaaagttgttccgttcttcgaagctaaggtggttgacctggacacaggaaaaaccctgggcgtcaaccagcgtggggaactctgcgtccgagggcccatgatcatgtctggctacgtgaacaaccccgaggccaccaatgccctcattgacaaggatggctggctccattcaggagacattgcctactgggacgaggatgaacacttttttattgtggacaggctcaagtcgcttatcaagtacaaaggctaccaggtggctcctgctgaattggaatccatcttacttcagcaccccaacatatttgatgcgggtgtggccggtctaccggatgatgatgcaggagagctgcccgctgctgttgttgtgctggagcatggtaagaccatgactgagaaggagattgtggactatgtagcgtctcaagtcacgaccgctaaaaaactaagagggggtgtggtctttgtggatgaggtcccaaaaggattgactgggaagctggatgctcgcaaaataagagaaatcctcatcaaagcaaagaagggagggaaaattgctgtctga

SEQ ID NO: 5 - DNA, 1032 bp, synthetic construct
  source 1..1032: mol_type = other DNA
atgtctaatctcctcactgtgcatcagaatcttccagctttaccggtagacgccacgtctgatgaagtgcgcaaaaatctcatggacatgttcagggaccggcaagccttcagtgagcacacatggaagatgttgttgtctgtgtgtcgctcctgggctgcctggtgcaaacttaacaacaggaagtggttccctgcagagcctgaggacgtcagagactatctgctctacttgcaagcacgaggactcgcggtaaagaccatccagcagcacctgggccagctgaacatgctgcacaggaggtctgggctgccccgaccaagtgactcaaatgctgtgtctctggtcatgagacgcatccgcaaggagaatgtggatgccggagaacgagccaagcaggctctggcttttgaacggacagactttgatcaggtgagatccctgatggagaactcagatagatgccaggacatccggaacctggcctttcttgggatagcctacaacaccttgctgaggatagcagaaattgccagaattcgggtcaaggacattagcaggacagatgggggcaggatgctcatccacattggccggactaaaacccttgtttcaactgcaggcgtggaaaaagccttgagcttaggtgtcaccaagctggtggagagatggatcagcgtctccggagttgcagacgacccaaataattatctcttctgtcgtgttcggaagaacggagttgcagcgccctcggctaccagccaactaagcacgagagctctggagggcatttttgaggccactcatcgcctgatctatggagcaaaagatgactccgggcagagatacctggcatggagtggtcatagtgctcgtgtcggtgctgcaagagatatggcccgggctggggtttccatacctgaaatcatgcaggctggtggctggacaaacgtgaacattgtgatgaactacatcaggaatctagattctgagacaggagccatggtgcgattactggaagatggcgattga

SEQ ID NO: 6 - DNA, 185 bp, synthetic construct
  source 1..185: mol_type = other DNA
tgataatagcaaacaccattgtcacactccagctggagcctcggtggccatgcttcttgccccttgggcccaaacaccattgtcacactccatccccccagcccctcctccccttcctgcacccgtaccccccaaacaccattgtcacactccagtggtctttgaataaagtctgagtgggcggc

SEQ ID NO: 7 - DNA, 4818 bp, synthetic construct
  source 1..4818: mol_type = other DNA
atgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctgaccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatggtgaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcattatcccccaccagatccacctgggagagctgcacgccattctgcggcggcagggcgatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggctgagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggcggccacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagggcaacagcgataagctgatcgccagaaagaaggactgggaccctaagaagtacggcggcttcaacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgtgctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcaaccggaagcaatacaacacgaccaaagaggtgctggacgccaccctgatccgtcagagcatcaccggcctgtacgagacacggatcgacctgtctcagctgggaggtgactctggcggctcaaaaagaaccgccgacggcagcgaattcgagcccaagaagaagaggaaagtctaa

SEQ ID NO: 8 - RNA, 20 nt, synthetic construct
  source 1..20: mol_type = other RNA
ttctccacaggagtcaggtg

SEQ ID NO: 9 - DNA, 18 bp, synthetic construct
  source 1..18: mol_type = other DNA
gactcctgtggagaagtc

SEQ ID NO: 10 - DNA, 18 bp, synthetic construct
  source 1..18: mol_type = other DNA
gactcctgcggagaagtc

(19) United States
(12) Patent Application Publication
(10) Pub. No.: US 2026/0308555 A1
(43) Pub. Date: Oct. 8, 2026

(54) Hematopoietic Stem Cell (HSC) Targeted Gene Editing and Methods of Use Thereof

(71) Applicants: The Trustees of the University of Pennsylvania, Philadelphia, PA (US); The Children's Hospital of Philadelphia, Philadelphia, PA (US)

(72) Inventors: Hamideh Parhiz, Gladwyne, PA (US); Tyler Papp, Philadelphia, PA (US); Stefano Rivella, Philadelphia, PA (US); Laura Breda, Philadelphia, PA (US); Michael Triebwasser, Philadelphia, PA (US)

(21) Appl. No.: 19/480,444

(22) PCT Filed: May 2, 2024
(86) PCT No.: PCT/US2024/027460

§ 371 Date: Oct. 31, 2025

Related U.S. Application Data
(60) Provisional application No. 63/499,580, filed on May 2, 2023.

(51) Int. Cl. C12N 9/22; A61P 7/00; C12N 15/11; C12N 15/88

(52) U.S. Cl. CPC C12N 9/226; A61P 7/00; C12N 15/11; C12N 15/88; C12N 2310/20

(57) ABSTRACT

The present invention relates to CD117 targeted LNP molecules for delivery of gene editing molecules to hematopoietic stem cells (HSCs) and methods of use thereof for base editing of genomic DNA for the treatment of a disease or disorder.

### CROSS-REFERENCE TO RELATED APPLICATIONS

This application claims the benefit of U.S. Provisional Patent Application No. 63/499,580, filed May 2, 2023, which is incorporated by reference herein in its entirety.

### STATEMENT REGARDING FEDERALLY SPONSORED RESEARCH

This invention was made with government support under AI045008 and HL007150 awarded by the National Institutes of Health. The government has certain rights in the invention.

### INCORPORATION BY REFERENCE OF A SEQUENCE LISTING SUBMITTED AS AN XML FILE

The present application hereby incorporates by reference the entire contents of the XML file named “046483-6267-00WO_SequenceListing.xml” which was created on Apr. 30, 2024, and is 18,261 bytes in size.

### BACKGROUND OF THE INVENTION

Hematopoietic stem cells (HSCs) divide throughout life, allowing them to give rise to the blood and immune system due to their self-renewal ability. Their multipotency enables the formation of myeloid (erythroid, megakaryocytic, and myeloid-immune) and lymphoid progenitors. HSCs are dependent on stromal-derived factors, including stem cell factor (SCF), which binds to the receptor c-Kit (CD117). CD117 is expressed on both short- and long-term HSCs and some hematopoietic progenitors (Kent, D., et al., 2008, Clin Cancer Res, 14:1926-1930). Replacement of diseased HSCs with healthy HSCs can cure non-malignant hematopoietic disorders (NMHD), such as hemoglobinopathies and immunodeficiencies. NMHD can be cured by allogeneic HSCT, but not all patients have a suitable immunologic match to minimize graft versus host disease (GVHD), a morbid, and potentially fatal, complication. Gene therapy can eliminate the risk of GVHD and cure NMHD by using autologous HSCs with the genetic defect corrected, either by gene addition or editing. Current hematopoietic gene therapy requires isolation of HSC from the body and ex vivo lentiviral transduction for gene addition or electroporation with purified reagents for genome editing. Gene modified HSC are then infused back to the patient. HSC elimination, i.e. conditioning, with chemotherapy or radiation is required to prepare a patient for HSCT. These conditioning procedures carry significant acute and chronic systemic toxicities, including infertility and secondary malignancies due to accumulated DNA damage. Some NMHD are due to DNA repair pathway mutations, such as radiosensitive severe combined immunodeficiency (SCID) or Fanconi anemia. These patients do not tolerate existing conditioning regimens due to excessive toxicity with alkylating chemotherapy or radiation, as well as increased rates of malignancy long-term.

Thus, there is a need in the art for improved targeted therapeutics for the treatment of diseases and disorders. The present invention addresses this need.

### SUMMARY OF THE INVENTION

In some embodiments, the invention relates to a composition for targeted delivery of gene editing agents to a target hematopoietic stem cell (HSC), the composition comprising a first delivery vehicle comprising an mRNA molecule encoding a Cas9 base editor and a second delivery vehicle comprising a short guide RNA (sgRNA), wherein each of the first and second delivery vehicle comprises a targeting moiety specific for CD117.

In some embodiments, each of the first and second delivery vehicles comprises a lipid nanoparticle (LNP).

In some embodiments, gene editing agents are encapsulated within the LNPs.

In some embodiments, the mRNA molecule encoding a Cas9 base editor encodes an adenine base editor (ABE) or a cytidine base editor (CBE). In some embodiments, the mRNA encoding the ABE is transcribed from a nucleic acid molecule comprising SEQ ID NO:7.

In some embodiments, the mRNA molecule is an isolated nucleoside-modified mRNA molecule. In some embodiments, at least one isolated nucleoside-modified RNA comprises pseudouridine or 1-methyl-pseudouridine.

In some embodiments, the targeting moiety of each of the first and second delivery vehicle comprises a CD117 antibody.

In some embodiments, the sgRNA is administered in excess of the Cas9 base editor.

In some embodiments, the invention relates to a method of treating a disease or disorder in a subject in need thereof, the method comprising administering a composition of composition for targeted delivery of gene editing agents to a target hematopoietic stem cell (HSC), the composition comprising a first delivery vehicle comprising an mRNA molecule encoding a Cas9 base editor and a second delivery vehicle comprising a short guide RNA (sgRNA), wherein each of the first and second delivery vehicle comprises a targeting moiety specific for binding to the HSC to the subject.

In some embodiments, the disease or disorder is a blood monogenic disorder, genetic defect, bone marrow genetic defect, cancer, platelet disorder, immunodeficiency, metabolic disease or autoimmune disease.

In some embodiments, the disease or disorder is a sickle cell disease. In some embodiments, the sickle cell disease is sickle cell anemia. In some embodiments, the sgRNA targets a hemoglobin subunit beta (HBB) gene. In some embodiments, the sgRNA comprises the nucleotide sequence of SEQ ID NO:8.

In some embodiments, the composition is administered by intravenous (IV), intraosseous infusion (IO), intraperitoneal (IP), intradermal, subcutaneous, inhalation, intranasal, or intramuscular delivery.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1, comprising FIG. 1A through FIG. 1G, depicts representative in vitro targeting of whole bone marrow or hematopoietic progenitors (Lin⁻) cells incubated with LNPs encapsulating luciferase (CD117/LNP-Luc) or Cre recombinase (CD117/LNP-Cre) mRNAs. FIG. 1A depicts representative luciferase activity normalized by total protein in whole bone marrow cells incubated with varying doses (indicated on x-axis) of targeted or control LNP-Luc for 18 hours in vitro (N=3). FIG. 1B depicts representative LNP-Luc treatment of Lineage negative (Lin⁻) bone marrow cells (N=3). FIG. 1C depicts representative assessment of ZsGreen⁺ reporter induction after 6-hour CD117/LNP-Cre treatment and 12-hour culture in Ai6 bone marrow (BM) cells triggered by removal of loxP flanked stop cassette by Cre. FIG. 1D depicts representative assessment of ZsGreen⁺ reporter induction after 6-hour CD117/LNP-Cre treatment and 12 hour culture in a Lin⁻Sca1⁺cKit⁺ (LKS) subset of Lin⁻ BM cells triggered by removal of loxP flanked stop cassette by Cre. FIG. 1E depicts representative assessment of ZsGreen⁺ reporter induction after 18-hour CD117/LNP-Cre treatment in Ai6 bone marrow (BM) cells triggered by removal of loxP flanked stop cassette by Cre. FIG. 1F depicts representative assessment of ZsGreen⁺ reporter induction after 18-hour CD117/LNP-Cre treatment in a Lin⁻Sca1⁺cKit⁺ (LKS) subset of Lin⁻ BM cells triggered by removal of loxP flanked stop cassette by Cre. FIG. 1G depicts representative assessment of ZsGreen⁺ reporter induction after 18-hour CD117/LNP-Cre treatment and 72-hour culture in Ai6 bone marrow (BM) cells triggered by removal of loxP flanked stop cassette by Cre. No difference between CD117/LNP-Cre editing in Lin⁻ cells treated with 0.1 and 0.5 or 0.5 and 1 μg; p>0.05, unpaired, two-sided t-test. All data represent mean and SD of 3 biological replicas. P values are from Dunnett's multiple comparison after two-way ANOVA (p<0.05). **p<0.01, **p<0.001, ****p<0.0001.

FIG. 2, comprising FIG. 2A through FIG. 2D, depicts representative delivery to antigen positive cells in whole bone marrow and viability in LNP-Cre treated bone marrow cells. FIG. 2A depicts representative luciferase activity normalized to protein in cell lysate and to the frequency of antigen positive cells in whole bone marrow (WBM) cells (anti-CD45 [85%], antiCD117 [2.8%], or unnormalized for control IgG/LNP. FIG. 2B depicts representative viability of Ai6 WBM cells after 6-hour treatment, and 12-hour culture, with in vitro exposure to increasing doses of LNP (up to 1 μg) and assessed by AO/PI staining. FIG. 2C depicts representative viability of Ai6 WBM cells after 18-hour treatment with in vitro exposure to increasing doses of LNP (up to 1 μg) and assessed by AO/PI staining. FIG. 2D depicts representative viability of Ai6 WBM cells after 18-hour treatment, and 12 hour culture, with in vitro exposure to increasing doses of LNP (up to 1 μg) and assessed by AO/PI staining. Shown are mean and SEM (N=3). P-value calculated with Dunnett's multiple comparison test after 2-way ANOVA (p<0.05). *p<0.05, **p<0.01, ***p<0.001, ****p<0.0001.

FIG. 3, comprising FIG. 3A through FIG. 3G, depicts representative results demonstrating CD117/LNP-Cre treatment ex vivo leads to superior tdTomato marking upon transplantation. FIG. 3A depicts representative percent tdTomato marking in myeloid (Gr1+) cells measured at 16 weeks post HSCT in lethally irradiated recipient congenic CD45.1 mice receiving Ai14 bone marrow treated ex vivo with 0.1 μg (n=5) and 1 μg (n=4) of control IgG/LNP-Cre, with 0.1 μg (n=5) and 1 μg (n=5) of CD117/LNP-Cre formulations. FIG. 3B depicts representative percent tdTomato marking in lymphoid (CD3+, left and B220+, right) cells measured at 16 weeks post HSCT in lethally irradiated recipient congenic CD45.1 mice receiving Ail4 bone marrow treated ex vivo with 0.1 μg (n=5) and 1 μg (n=4) of control IgG/LNP-Cre, with 0.1 μg (n=5) and 1 μg (n=5) of CD117/LNP-Cre formulations. FIG. 3C depicts representative kinetic analysis of erythroid editing measured up to 16 weeks post HSCT. Mean and SEM shown. FIG. 3D depicts representative tdTomato marking in in the bone marrow (BM) and BM subsets: c-Kit⁺ (Lin⁻c-Kit⁺), LSK (Lin⁻c-Kit⁺Sca1⁺), and LT-HSC (LSK CD150⁺CD48⁻). FIG. 3E depicts a representative colony forming unit assay from Ai14 bone marrow treated ex vivo with 0.1 μg or 1 μg of control IgG/LNP-Cre or CD117/LNP-Cre formulations or untreated. FIG. 3F depicts representative semi-quantitative PCR of bone marrow genomic DNA isolates from the groups in FIG. 1A through FIG. 1C at 4-months post-BMT. FIG. 3F depicts representative semi-quantitative PCR of spleen genomic DNA isolates from the groups in FIG. 1A through FIG. 1C at 4-months post-BMT. **271 bp Cre-recombinase edited gDNA region, *1142 bp unedited region are indicated. For FIG. 3A and FIG. 3B, mean and SEM are shown. P-values are Tukey's multiple comparison test after one-way ANOVA (p<0.05). ***p<0.001, ****p<0.0001.

FIG. 4, comprising FIG. 4A through FIG. 4F, depicts representative results demonstrating ex vivo CD117-LNP/Cre treated HSC retain multi-lineage engraftment. FIG. 4A depicts representative peripheral blood donor chimerism in lethally irradiated congenic (CD45.1) recipients of ex vivo treated of bone marrow cells from Ai14 donor mice (CD45.2) with CD117/LNP-Cre (left) or Control IgG/LNP-Cre (right) across a 100-fold range in dose. FIG. 4B depicts representative tdTomato+ cell frequency in red blood cells (RBC), white blood cells (CD45.2⁺), and granulocytes (Gr1⁺) cells from 8-16 weeks post-transplant after ex vivo treatment of Ai14 bone marrow cells with CD117/LNP-Cre. FIG. 4C depicts representative tdTomato+ cell frequency in red blood cells (RBC), white blood cells (CD45.2+), and granulocytes (Gr1+) cells from 8-16 weeks post-transplant after ex vivo treatment of Ai14 bone marrow cells with control IgG/LNP-Cre. FIG. 4D depicts representative tdTomato⁺ cell frequency in peripheral blood myeloid (Gr1+) and lymphoid cells (CD3+ [T-cells], B220+ [B-cells]) and in bone marrow (BM) subsets (c-Kit, Lin⁻c-Kit⁺ subset, LSK, Lin⁻c-Kit⁺Sca1⁺, SLAM, LSK CD150⁺CD48⁻) at 4 months after 0.01 or 0.05 mg of CD117/LNP-Cre or control IgG/LNP-Cre. FIG. 4E depicts a representative colony forming unit assay from bone marrow at 4 months after transplantation with ex vivo treated Ai14 BM at the doses shown. FIG. 4F depicts representative percentages of total CFU that are tdTomato+ 16 weeks post-transplant with ex vivo treated BM by quantification of the panels in FIG. 4E; number of colonies for Control IgG 1 μg N=81, 0.1 μg N=98, 0.05 μg N=76, 0.01 μg N=79 and CD117/LNP-Cre 1 μg N=77, 0.1 μg N=94, 0.05 μg N=96, 0.01 μg N=92.

FIG. 5, comprising FIG. 5A through FIG. 5E, depicts representative results demonstrating that ex vivo CD117/LNP-Cre edited HSC persist upon secondary transplantation. FIG. 5A depicts representative tdTomato⁺ cell frequency in peripheral red blood cells four months after transplantation with bone marrow from primary chimeras of ex vivo CD117 or control IgG/LNP-Cre (0.01 mg mRNA) treated bone marrow. FIG. 5A depicts representative tdTomato⁺ cell frequency in peripheral blood myeloid cells (Gr1+) four months after transplantation with bone marrow from primary chimeras of ex vivo CD117 or control IgG/LNP-Cre (0.01 mg mRNA) treated bone marrow. FIG. 5A depicts representative tdTomato⁺ cell frequency in peripheral blood lymphoid cells (CD3+ [T-cells], B220+ [B-cells]) four months after transplantation with bone marrow from primary chimeras of ex vivo CD117 or control IgG/LNP-Cre (0.01 mg mRNA) treated bone marrow. FIG. 5D depicts representative donor chimerism in peripheral blood at 4 months in secondary transplants. FIG. 5E depicts representatitve frequency of gene edited cells in whole bone marrow (BM) and bone marrow subsets (c-Kit, Lin⁻c-Kit⁺ subset, LSK, Lin⁻c-Kit⁺Sca1⁺, SLAM, LSK CD150⁺CD48⁻) in secondary chimeras at 16 weeks posttransplant.

FIG. 6, comprising FIG. 6A through FIG. 6L, CD117/LNP-Cre formulations lead to over 50% tdTomato marking in LT-HSC after in vivo injection. FIG. 6A depicts representative biodistribution of i.v. injection of 1 μg of targeted LNP-mRNA expression in vivo by luminescence imaging at 24 hours. FIG. 6B depicts a representative sample set of dissected mouse organs were analyzed 5 min after the administration of D-luciferin. tdTomato⁺ cell frequency in peripheral blood myeloid (Gr1+) cells at 4 months after 5 μg of CD117/LNP-Cre. FIG. 6C depicts a representative sample set of dissected mouse organs were analyzed 5 min after the administration of D-luciferin. tdTomato⁺ cell frequency in peripheral blood lymphoid cells (CD3+ [T-cells], B220+ [B-cells]) at 4 months after 5 μg of CD117/LNP-Cre. FIG. 6B depicts a representative sample set of dissected mouse organs were analyzed 5 min after the administration of D-luciferin. tdTomato⁺ cell frequency in bone marrow (BM) subsets (c-Kit, Lin⁻c-Kit⁺ subset, LSK, Lin⁻c-Kit⁺Sca1⁺, SLAM/LT-HSC, LSK CD150⁺CD48⁻) at 4 months after 5 μg of CD117/LNP-Cre. FIG. 6E depicts representative tdTomato⁺ cell frequency in peripheral blood myeloid cells at 4 months after 5 or 1 μg of CD117/LNP-Cre. FIG. 6E depicts representative tdTomato⁺ cell frequency in peripheral lymphoid cells at 4 months after 5 or 1 μg of CD117/LNP-Cre. FIG. 6E depicts representative tdTomato⁺ cell frequency in bone marrow subsets at 4 months after 5 or 1 μg of CD117/LNP-Cre. FIG. 6H depicts representative edited RBC frequency over time in Ai9 mice treated in vivo with 5 μg of CD117/LNP-Cre or control IgG/LNP-Cre (N=5 both groups). FIG. 6I depicts representative edited RBC frequency over time in Ai9 mice treated in vivo with 1 or 5 μg of CD117/LNP-Cre (N=5 and 7, respectively). FIG. 6J depicts a representative colony forming unit assay from bone marrow at 4 months after in vivo treatment with 5 μg control-IgG/LNP-Cre (top), no treatment (middle), or 5 μg CD117/LNP-Cre (bottom). FIG. 6K depicts representative semi-quantitative PCR of bone marrow genomic DNA isolates from the groups in A-C at 4 months post BMT. FIG. 6L depicts representative semi-quantitative PCR of spleen genomic DNA isolates from the groups in A-C at 4 months post BMT. **271 bp Cre-recombinase edited gDNA region, *1142 bp unedited region are indicated. For FIG. 6B through FIG. 6G, mean and SEM of the shown P-values are reported from paired t-test. ***p<0.001.

FIG. 7, comprising FIG. 7A through FIG. 7E, depicts representative in vivo editing after CD117/LNP treatment. FIG. 7A depicts representative percentage of total CFU that are tdTomato+ at 4 months post IV treatment. Number of colonies for control IgG 5 μg N=182, CD117/LNP-Cre 5 μg N=129, and untreated N=265; two independent wells counted. FIG. 7B depicts representative gene editing frequency in non-hematopoietic organs/cells of the liver 16 weeks post in vivo treatment with CD117/LNP-Cre (1 μg and 5 μg) and control IgG/LNP-Cre (5 μg) assessment by flow cytometry. FIG. 7C depicts representative gene editing frequency in non-hematopoietic organs/cells of the lung 16 weeks post in vivo treatment with CD117/LNP-Cre (1 μg and 5 μg) and control IgG/LNP-Cre (5 μg) assessment by flow cytometry. FIG. 7D depicts representative frequency of cKit⁺ cells in the lung and frequency of gene editing in those cKit⁺ lung cells 6d after in vivo CD117/LNP-Cre treatment (3.5 μg) N=5. FIG. 7E depicts representative gene editing frequency in non-hematopoietic organs/cells of the testis 16 weeks post in vivo treatment with CD117/LNP-Cre (5 μg and 1 μg) and control IgG/LNP-Cre (5 μg) assessment by flow cytometry. For FIG. 7B through FIG. 7E, data are shown as mean and standard deviation (N=5 to 7). For FIG. 7B and FIG. 7C, p-value calculated using Tukey's multiple comparison test after one-way ANOVA (p<0.05), ****P<0.0001; comparisons to control liver not shown. For FIGS. 7D and 7E, p-value calculated using Dunn's multiple comparison test after one-way ANOVA (p<0.05).

FIG. 8, comprising FIG. 8A through FIG. 8E, depicts representative results demonstrating CD117/LNP-Cre edited HSC persist upon primary transplantation of BM from in vivo treated mice. FIG. 8A depicts representative tdTomato⁺ cell frequency in peripheral blood red blood cells 4 months after transplantation with bone marrow from Ai9 mice injected in vivo with control IgG/LNP-Cre or CD117/LNPCre formulations. FIG. 8B depicts representative tdTomato⁺ cell frequency in peripheral myeloid cells (Gr1+) 4 months after transplantation with bone marrow from Ai9 mice injected in vivo with control IgG/LNP-Cre or CD117/LNPCre formulations. FIG. 8C depicts representative tdTomato⁺ cell frequency in peripheral blood lymphoid cells (CD3+ [T-cells], B220+ [B-cells]) 4 months after transplantation with bone marrow from Ai9 mice injected in vivo with control IgG/LNP-Cre or CD117/LNPCre formulations. FIG. 7D depicts representative donor chimerism in the primary chimeras at 16 weeks posttransplant. FIG. 8E depicts representative frequency of gene edited cells in whole bone marrow (BM) and bone marrow subsets (c-Kit, Lin⁻c-Kit⁺ subset, LSK, Lin⁻c-Kit⁺Sca1⁺, SLAM, LSK CD150⁺CD48⁻) in primary chimeras at 16 weeks posttransplant. For FIG. 8A through FIG. 8E, data is shown as mean and standard deviation, P-value calculated by Mann-Whitney test, **p<0.01 ***p<0.001.

FIG. 9, comprising FIG. 9A through FIG. 9D, depicts representative analyses of genome editing, cell viability and proliferation in human erythroid cells treated with CD117/LNP-ABE and CD117/LNP-sgRNA. FIG. 9A depicts representative sequences of C editing of control unedited (top) and edited (bottom) genomic DNA extracted from SCD cells after treatment with anti-human CD117/LNP formulations carrying an adenine base editor and a sgRNA aimed at converting the pathogenic codon 6 (highlighted in blue, GAG,) to non-pathogenic variant (->GCG) named HBB^{G}-Makassar. FIG. 9B depicts quantification of C editing in as in FIG. 9A. FIG. 9C depicts representative quantification of viability (AOPI staining) from untreated (n=4) and treated (n=7) early erythroid progenitors cultured independently. FIG. 9D depicts proliferation rate of specimens in FIG. 9C calculated by measuring the cell count fold increase from DO to D7/8 in differentiation media.

FIG. 10, comprising FIG. 10A through FIG. 10D, depicts representative base editing of the E6V sickle cell mutation with Human CD117 targeted LNP. FIG. 10A depicts representative reverse-phase (RP) HPLC of in vitro differentiated sickle cell disease affected erythroid progenitors after treatment with anti-human CD117 (hCD117)/LNP-NRCH cas9 ABE-8e mRNA and hCD117/LNP gRNA. Base editing yields non-pathogenic Hbb^{G-Makassar} (b^{G}), which elutes before Hbb^{S} (pathogenic, b^{S}) and the α-globin protein (a). % shown is b^{G}/(b^{G}+b^{S})*100. Two doses of LNP are shown. FIG. 10B depicts representative images of sickling of in vitro differentiated erythroid progenitors under hypoxic conditions at the treatments in (A). Arrowheads indicate sickled morphology. Scale bar 20 microns. FIG. 10C depicts representative sickled cells from unedited (N=10) and edited (N=30, varying mRNA doses) sickling assays. FIG. 10D depicts representative correlation of % b^{G} by RP-HPLC (protein) to base edited allele frequency (DNA).

FIG. 11, comprising FIG. 11A through FIG. 11C, depicts representative imaging of mice and livers upon i.v. administration of CD117/LNP-Luc versus CD117/LNP-Luc-miRt formulations. FIG. 11A depicts representative biodistribution upon i.v. injection of 1 μg of targeted LNP-mRNA expression in mice in vivo by luminescence imaging at 24 hours. FIG. 11B depicts representative biodistribution in livers dissected from animals of FIG. 11A analyzed 5 min after the administration of D-luciferin. FIG. 11C depicts representative quantification of bioluminescence.

### DETAILED DESCRIPTION

The present invention relates to compositions comprising CD117 targeted LNP molecules comprising at least one RNA molecule for genomic editing of Hematopoietic stem cells (HSCs). In some embodiments, the composition relates to a combination of a first CD117-targed LNP comprising an mRNA molecule encoding a base-editing molecule and a second CD117-targed LNP comprising a guide RNA for targeting of the base editing molecule to a target base. In some embodiments, the target base is a disease-associated single nucleotide polymorphism (SNP), and the base editing method of the invention serves to revert the disease-associated mutation to a “wild-type,” or non-disease associated, base.

The present invention also relates to methods of use of the compositions described herein for genetic editing of HSCs as well as methods of treating diseases or disorders in subjects including, but not limited to, monogenic disorders, non-hematopoietic diseases and bone marrow genetic defects. Exemplary monogenic disorders include, but are not limited to, non-malignant hematopoietic disorder (NMHD) such as hemoglobinopathies, congenital anemias or thrombocytopenias, and immunodeficiencies. Exemplary non-hematopoietic diseases include, but are not limited to, cystic fibrosis, metabolic disorders, and myopathies. Exemplary bone marrow genetic defects include, but are not limited to, leukemia, aplastic anemia, myeloproliferative disorders, an inherited bone marrow failure syndrome (IBMFS) such as Fanconi anemia, dyskeratosis congenital, Shwachman-Diamond syndrome, Diamond-Blackfan anemia, severe congenital neutropenia, a primary immunodeficiency such as X1-SCID and Wiskott-Aldrich syndrome, an erythroid disorder such as sickle cell disease (SCD), pyruvate kinase deficiency, or a lysosomal storage diseases such as Fabry disease and Pompe disease.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “adjuvant” as used herein means an agent that modifies or boosts the strength and longevity of a desired therapeutic response, and/or broadens the therapeutic response to a concomitantly administered agent.

The term “antibody,” as used herein, refers to an immunoglobulin molecule, which specifically binds with an antigen or epitope. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic-specificity determining variable regions of an intact antibody. Examples of antibody fragments include, but are not limited to, Fab, Fab′, F(ab′) 2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. k and 1 light chains refer to the two major antibody light chain isotypes.

By the term “synthetic antibody” as used herein, is meant an antibody, which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned) or other technology, which is available and well known in the art.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

The term “physiologically effective dosage” refers to an amount of an agent that produces a measurable biologic or physiologic effect in the recipient subject that is related to the activity of the agent(s). The physiologically effective dosage will vary depending on the compound, the age, weight, etc., of the subject being administered the agent, and the biologic or physiologic effect being measured.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or CDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) RNA, and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleosides (nucleobase bound to ribose or deoxyribose sugar via N-glycosidic linkage) are used. “A” refers to adenosine, “C” refers to cytidine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. In addition, the nucleotide sequence may contain modified nucleosides that are capable of being translation by translational machinery in a cell. For example, an mRNA where all of the uridines have been replaced with pseudouridine, 1-methyl pseudouridine, or another modified nucleoside.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA or RNA sequences are contiguous and, where necessary to join two protein coding regions, in the same reading frame.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In certain instances, the polynucleotide or nucleic acid of the invention is a “nucleoside-modified nucleic acid,” which refers to a nucleic acid comprising at least one modified nucleoside. A “modified nucleoside” refers to a nucleoside with a modification. For example, over one hundred different nucleoside modifications have been identified in RNA (Rozenski, et al., 1999, The RNA Modification Database: 1999 update. Nucl Acids Res 27:196-197).

In certain embodiments, “pseudouridine” refers, in another embodiment, to m^{1a} cp³Y (1-methyl-3-(3-amino-3-carboxypropyl) pseudouridine. In another embodiment, the term refers to m¹Y (1-methylpseudouridine). In another embodiment, the term refers to Ym (2′-O-methylpseudouridine. In another embodiment, the term refers to m⁵D (5-methyldihydrouridine). In another embodiment, the term refers to m³Y (3-methylpseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. Each possibility represents a separate embodiment of the present invention.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence. For example, the promoter that is recognized by bacteriophage RNA polymerase and is used to generate the mRNA by in vitro transcription.

By the term “specifically binds,” as used herein with respect to an affinity ligand, in particular, an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “therapeutic” as used herein means a treatment and/or prophylaxis. A therapeutic effect is obtained by suppression, diminution, remission, or eradication of at least one sign or symptom of a disease or disorder.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Alkyl” refers to a straight or branched hydrocarbon chain radical consisting solely of carbon and hydrogen atoms, which is saturated or unsaturated (i.e., contains one or more double and/or triple bonds), having from one to twenty-four carbon atoms (C₁-C₂₄ alkyl), one to twelve carbon atoms (C₁-C₁₂ alkyl), one to eight carbon atoms (C₁-C₈alkyl) or one to six carbon atoms (C₁-C₆ alkyl) and which is attached to the rest of the molecule by a single bond, e.g., methyl, ethyl, n propyl, 1-methylethyl (iso propyl), n butyl, n pentyl, 1,1 dimethylethyl (t butyl), 3 methylhexyl, 2 methylhexyl, ethenyl, prop 1 enyl, but-1-enyl, pent-1-enyl, penta-1,4-dienyl, ethynyl, propynyl, butynyl, pentynyl, hexynyl, and the like. Unless specifically stated otherwise, an alkyl group is optionally substituted.

“Alkylene” or “alkylene chain” refers to a straight or branched divalent hydrocarbon chain linking the rest of the molecule to a radical group, consisting solely of carbon and hydrogen, which is saturated or unsaturated (i.e., contains one or more double (alkenylene) and/or triple bonds (alkynylene)), and having, for example, from one to twenty-four carbon atoms (C₁-C₂₄ alkylene), one to fifteen carbon atoms (C₁-C₁₅ alkylene), one to twelve carbon atoms (C₁-C₁₂ alkylene), one to eight carbon atoms (C₁-C₈ alkylene), one to six carbon atoms (C₁-C₆ alkylene), two to four carbon atoms (C₂-C₄ alkylene), one to two carbon atoms (C₁-C₂ alkylene), e.g., methylene, ethylene, propylene, n-butylene, ethenylene, propenylene, n-butenylene, propynylene, n-butynylene, and the like. The alkylene chain is attached to the rest of the molecule through a single or double bond and to the radical group through a single or double bond. The points of attachment of the alkylene chain to the rest of the molecule and to the radical group can be through one carbon or any two carbons within the chain. Unless stated otherwise specifically in the specification, an alkylene chain may be optionally substituted.

“Cycloalkyl” or “carbocyclic ring” refers to a stable non aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7 dimethyl bicyclo[2.2.1]heptanyl, and the like. Unless specifically stated otherwise, a cycloalkyl group is optionally substituted.

“Cycloalkylene” is a divalent cycloalkyl group. Unless otherwise stated specifically in the specification, a cycloalkylene group may be optionally substituted.

“Heterocyclyl” or “heterocyclic ring” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless specifically stated otherwise, a heterocyclyl group may be optionally substituted.

The term “substituted” used herein means any of the above groups (e.g., alkyl, cycloalkyl or heterocyclyl) wherein at least one hydrogen atom is replaced by a bond to a non-hydrogen atoms such as, but not limited to: a halogen atom such as F, Cl, Br, and I; oxo groups (═O); hydroxyl groups (—OH); alkoxy groups (—OR^{a}, where R^{a} is C₁-C₁₂ alkyl or cycloalkyl); carboxyl groups (—OC(═O)R^{a} or —C(═O)OR^{a}, where R^{a} is H, C₁-C₁₂ alkyl or cycloalkyl); amine groups (—NR^{a}R^{b}, where Rₐ and R^{b} are each independently H, C₁-C₁₂ alkyl or cycloalkyl); C₁-C₁₂ alkyl groups; and cycloalkyl groups. In some embodiments the substituent is a C₁-C₁₂ alkyl group. In other embodiments, the substituent is a cycloalkyl group. In other embodiments, the substituent is a halo group, such as fluoro. In other embodiments, the substituent is a oxo group. In other embodiments, the substituent is a hydroxyl group. In other embodiments, the substituent is an alkoxy group. In other embodiments, the substituent is a carboxyl group. In other embodiments, the substituent is an amine group.

“Optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

### Agents

In some embodiments, the CD117-LNP comprises an mRNA molecule encoding a base editing protein. Exemplary base editing proteins that can be encoded by the mRNA molecule and delivered to HSC according to the methods of the invention include, but are not limited to, an adenine base editor (ABE), a cytidine base editor (CBE), a CRISPR based nucleic acid editor (e.g., Cas9), and other nucleic acid editing proteins or protein domains, e.g., deaminase domains, polymerase domains, and/or base excision enzymes.

In some embodiments, the compositions of the invention provide a CD117-LNP comprising an mRNA molecule encoding a base editor, wherein the base editor when in association with a guide RNA (gRNA) specifically edits a target base. In some embodiments, the target base is a disease-associated base. Therefore, in some embodiments, the compositions of the invention provide a combination of a CD117-LNP comprising an mRNA molecule encoding a base editor and a CD117-LNP comprising a gRNA for targeted base editing.

mRNA molecules include nucleotide oligomers containing modified backbones or non-natural inter-nucleoside linkages. Oligomers having modified backbones include those that retain a phosphorus atom in the backbone and those that do not have a phosphorus atom in the backbone. For the purposes of this disclosure, modified oligonucleotides that do not have a phosphorus atom in their internucleoside backbone are also considered to be nucleotide oligomers. Nucleotide oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriest-ers, and boranophosphates. Various salts, mixed salts and free acid forms are also included.

### In Vitro Transcribed RNA

In some embodiments, the composition of the invention comprises in vitro transcribed (IVT) RNA. In some embodiments, the composition of the invention comprises IVT RNA encoding a therapeutic protein. In some embodiments, the composition of the invention comprises IVT RNA encoding a plurality of therapeutic proteins.

In some embodiments, an IVT RNA can be introduced to a cell as a form of transient transfection. The RNA is produced by in vitro transcription using a plasmid DNA template generated synthetically. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. In some embodiments, the desired template for in vitro transcription is a therapeutic protein, as described elsewhere herein.

In some embodiments, the DNA to be used for PCR contains an open reading frame. The DNA can be from a naturally occurring DNA sequence from the genome of an organism. In some embodiments, the DNA is a full-length gene of interest of a portion of a gene. The gene can include some or all of the 5′ and/or 3′ untranslated regions (UTRs). The gene can include exons and introns. In some embodiments, the DNA to be used for PCR is a human gene. In another embodiment, the DNA to be used for PCR is a human gene including the 5′ and 3′ UTRs. In another embodiment, the DNA to be used for PCR is a gene from a pathogenic or commensal organism, including bacteria, viruses, parasites, and fungi. In another embodiment, the DNA to be used for PCR is from a pathogenic or commensal organism, including bacteria, viruses, parasites, and fungi, including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

Genes that can be used as sources of DNA for PCR include genes that encode a gene editing protein or a variant thereof. In some embodiments, the gene editing protein is a CRISPR based nucleic acid editor (e.g., a Cas9 adenine base editor (ABE) or a Cas9 cytidine base editor (CBE), or other nucleic acid editing proteins or protein domains, e.g., deaminase domains, polymerase domains, and/or base excision enzymes. In various embodiments, a plasmid is used to generate a template for in vitro transcription of RNA which is used for transfection.

Chemical structures with the ability to promote stability and/or translation efficiency may also be used. In some embodiments, the RNA has 5′ and 3′ UTRs. In some embodiments, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In some embodiments, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the RNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In some embodiments, the promoter is a T7 RNA polymerase promoter, as described elsewhere herein. Other useful promoters include, but are not limited to, T3 and SP6 RNA polymerase promoters. Consensus nucleotide sequences for T7, T3 and SP6 promoters are known in the art.

In some embodiments, the RNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized RNA which is effective in eukaryotic transfection when it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which can be ameliorated through the use of recombination incompetent bacterial cells for plasmid propagation.

Poly(A) tails of RNAs can be further extended following in vitro transcription with the use of a poly(A) polymerase, such as E. coli polyA polymerase (E-PAP) or yeast polyA polymerase. In some embodiments, increasing the length of a poly(A) tail from 100 nucleotides to between 300 and 400 nucleotides results in about a two-fold increase in the translation efficiency of the RNA. Additionally, the attachment of different chemical groups to the 3′ end can increase RNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In some embodiments, RNAs produced by the methods to include a 5′ cap1 structure. Such cap1 structure can be generated using Vaccinia capping enzyme and 2′-O-methyltransferase enzymes (CellScript, Madison, WI). Alternatively, 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

### Nucleoside-Modified RNA

In some embodiments, the composition of the present invention comprises a nucleoside-modified nucleic acid. In some embodiments, the composition of the invention comprises a nucleoside-modified RNA encoding a protein for genetic modification. In some embodiments, the composition of the invention comprises a nucleoside-modified RNA encoding a protein for base editing. In some embodiments, the composition of the invention comprises a gRNA molecule specific for binding to a target nucleic acid molecule.

For example, In some embodiments, the composition comprises one or more nucleoside-modified RNA. In some embodiments, the composition comprises a nucleoside-modified mRNA encoding a protein for genetic modification. Nucleoside-modified mRNA have particular advantages over non-modified mRNA, including for example, increased stability, low or absent innate immunogenicity, and enhanced translation. Nucleoside-modified mRNA useful in the present invention is further described in U.S. Pat. No. 8,278,036, which is incorporated by reference herein in its entirety.

In certain instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects (Kromminga and Schellekens, 2005, Ann NY Acad Sci 1050:257-265).

In certain embodiments, the nucleoside-modified RNA comprises the naturally occurring modified-nucleoside pseudouridine. In certain embodiments, inclusion of pseudouridine makes the mRNA more stable, non-immunogenic, and highly translatable (Kariko et al., 2008, Mol Ther 16:1833-1840; Anderson et al., 2010, Nucleic Acids Res 38:5884-5892; Anderson et al., 2011, Nucleic Acids Research 39:9329-9338; Kariko et al., 2011, Nucleic Acids Research 39:e142; Kariko et al., 2012, Mol Ther 20:948-953; Kariko et al., 2005, Immunity 23:165-175).

It has been demonstrated that the presence of modified nucleosides, including pseudouridines in RNA suppress their innate immunogenicity (Kariko et al., 2005, Immunity 23:165-175). Further, protein-encoding, in vitro-transcribed RNA containing pseudouridine can be translated more efficiently than RNA containing no or other modified nucleosides (Kariko et al., 2008, Mol Ther 16:1833-1840). Subsequently, it is shown that the presence of pseudouridine improves the stability of RNA (Anderson et al., 2011, Nucleic Acids Research 39:9329-9338) and abates both activation of PKR and inhibition of translation (Anderson et al., 2010, Nucleic Acids Res 38:5884-5892). A preparative HPLC purification procedure has been established that was critical to obtain pseudouridine-containing RNA that has superior translational potential and no innate immunogenicity (Kariko et al., 2011, Nucleic Acids Research 39:e142). Administering HPLC-purified, pseudourine-containing RNA coding for erythropoietin into mice and macaques resulted in a significant increase of serum EPO levels (Kariko et al., 2012, Mol Ther 20:948-953), thus confirming that pseudouridine-containing mRNA is suitable for in vivo protein therapy.

The present invention encompasses RNA, oligoribonucleotide, and polyribonucleotide molecules comprising pseudouridine or a modified nucleoside. In certain embodiments, the composition comprises an isolated nucleic acid, wherein the nucleic acid comprises a pseudouridine or a modified nucleoside. In certain embodiments, the composition comprises a vector, comprising an isolated nucleic acid, wherein the nucleic acid comprises a pseudouridine or a modified nucleoside.

In some embodiments, the nucleoside-modified RNA of the invention is IVT RNA, as described elsewhere herein. For example, in certain embodiments, the nucleoside-modified RNA is synthesized by T7 phage RNA polymerase. In another embodiment, the nucleoside-modified mRNA is synthesized by SP6 phage RNA polymerase. In another embodiment, the nucleoside-modified RNA is synthesized by T3 phage RNA polymerase.

In some embodiments, the modified nucleoside is m^{1a} cp³Ψ (1-methyl-3-(3-amino-3-carboxypropyl) pseudouridine. In another embodiment, the modified nucleoside is m¹Ψ (1-methylpseudouridine). In another embodiment, the modified nucleoside is Ψm (2′-O-methylpseudouridine. In another embodiment, the modified nucleoside is m⁵D (5-methyldihydrouridine). In another embodiment, the modified nucleoside is m³Ψ (3-methylpseudouridine). In another embodiment, the modified nucleoside is a pseudouridine moiety that is not further modified. In another embodiment, the modified nucleoside is a monophosphate, diphosphate, or triphosphate of any of the above pseudouridines. In another embodiment, the modified nucleoside is any other pseudouridine-like nucleoside known in the art.

In another embodiment, the nucleoside that is modified in the nucleoside-modified RNA the present invention is uridine (U). In another embodiment, the modified nucleoside is cytidine (C). In another embodiment, the modified nucleoside is adenosine (A). In another embodiment, the modified nucleoside is guanosine (G).

In another embodiment, the modified nucleoside of the present invention is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is Ψ (pseudouridine). In another embodiment, the modified nucleoside is Um (2′-O-methyluridine).

In other embodiments, the modified nucleoside is m¹A (1-methyladenosine); m²A (2-methyladenosine); Am (2′-O-methyladenosine); ms²m⁶A (2-methylthio-N⁶-methyladenosine); i⁶A (N⁶-isopentenyladenosine); ms²i⁶A (2-methylthio-N⁶isopentenyladenosine); io⁶A (N⁶-(cis-hydroxyisopentenyl) adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A (N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²₂G (N²,N²-dimethylguanosine); m²Gm (N²,2′-O-dimethylguanosine); m²₂Gm (N²,N²,2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl) uridine); ho⁵U (5-hydroxyuridine); moSU (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵s²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶₂A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N²,2′-O-dimethyladenosine); m⁶₂Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine).

In another embodiment, a nucleoside-modified RNA of the present invention comprises a combination of 2 or more of the above modifications. In another embodiment, the nucleoside-modified RNA comprises a combination of 3 or more of the above modifications. In another embodiment, the nucleoside-modified RNA comprises a combination of more than 3 of the above modifications.

In another embodiment, between 0.1% and 100% of the residues in the nucleoside-modified of the present invention are modified (e.g. either by the presence of pseudouridine or a modified nucleoside base). In another embodiment, 0.1% of the residues are modified. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 8%. In another embodiment, the fraction is less than 10%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, 0.1% of the residues of a given nucleoside (i.e., uridine, cytidine, guanosine, or adenosine) are modified. In another embodiment, the fraction of the given nucleotide that is modified is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction of the given nucleotide that is modified is less than 8%. In another embodiment, the fraction is less than 10%. In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, a nucleoside-modified RNA of the present invention is translated in the cell more efficiently than an unmodified RNA molecule with the same sequence. In another embodiment, the nucleoside-modified RNA exhibits enhanced ability to be translated by a target cell. In another embodiment, translation is enhanced by a factor of 2-fold relative to its unmodified counterpart. In another embodiment, translation is enhanced by a 3-fold factor. In another embodiment, translation is enhanced by a 5-fold factor. In another embodiment, translation is enhanced by a 7-fold factor. In another embodiment, translation is enhanced by a 10-fold factor. In another embodiment, translation is enhanced by a 15-fold factor. In another embodiment, translation is enhanced by a 20-fold factor. In another embodiment, translation is enhanced by a 50-fold factor. In another embodiment, translation is enhanced by a 100-fold factor. In another embodiment, translation is enhanced by a 200-fold factor. In another embodiment, translation is enhanced by a 500-fold factor. In another embodiment, translation is enhanced by a 1000-fold factor. In another embodiment, translation is enhanced by a 2000-fold factor. In another embodiment, the factor is 10-1000-fold. In another embodiment, the factor is 10-100-fold. In another embodiment, the factor is 10-200-fold. In another embodiment, the factor is 10-300-fold. In another embodiment, the factor is 10-500-fold. In another embodiment, the factor is 20-1000-fold. In another embodiment, the factor is 30-1000-fold. In another embodiment, the factor is 50-1000-fold. In another embodiment, the factor is 100-1000-fold. In another embodiment, the factor is 200-1000-fold. In another embodiment, translation is enhanced by any other significant amount or range of amounts.

### Delivery Vehicle

In some embodiments, the delivery vehicle is a colloidal dispersion system, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

The use of lipid formulations is contemplated for the introduction of the at least one agent into a host cell (in vitro, ex vivo or in vivo). In another aspect, the at least one agent may be associated with a lipid. The at least one agent associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/nucleic acid or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Chol”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5:505-10). However, compositions that have different structures in solution than the normal vesicular structure are also encompassed. For example, the lipids may assume a micellar structure or merely exist as nonuniform aggregates of lipid molecules. Also contemplated are lipofectamine-agent complexes.

In some embodiments, delivery of the at least one agent comprises any suitable delivery method, including exemplary delivery methods described elsewhere herein. In certain embodiments, delivery of the at least one agent to a subject comprises mixing the at least one agent with a transfection reagent prior to the step of contacting. In another embodiment, a method of the present invention further comprises administering the at least one agent together with the transfection reagent. In another embodiment, the transfection reagent is a cationic lipid reagent.

In another embodiment, the transfection reagent is a lipid-based transfection reagent. In another embodiment, the transfection reagent is a protein-based transfection reagent. In another embodiment, the transfection reagent is a polyethyleneimine based transfection reagent. In another embodiment, the transfection reagent is calcium phosphate. In another embodiment, the transfection reagent is Lipofectin®, Lipofectamine®, or TransIT®. In another embodiment, the transfection reagent is any other transfection reagent known in the art.

In another embodiment, the transfection reagent forms a liposome. Liposomes, in another embodiment, increase intracellular stability, increase uptake efficiency and improve biological activity. In another embodiment, liposomes are hollow spherical vesicles composed of lipids arranged in a similar fashion as those lipids which make up the cell membrane. In some embodiments, the liposomes comprise an internal aqueous space for entrapping water-soluble compounds. In another embodiment, liposomes can deliver the at least one agent to cells in an active form.

In some embodiments, the composition comprises a lipid nanoparticle (LNP) and at least one agent.

The term “lipid nanoparticle” refers to a particle having at least one dimension on the order of nanometers (e.g., 1-1,000 nm) which includes one or more lipids. In various embodiments, the particle includes a lipid of Formula (I), (II) or (III). In some embodiments, lipid nanoparticles are included in a formulation comprising at least one agent as described herein. In some embodiments, such lipid nanoparticles comprise a cationic lipid (e.g., a lipid of Formula (I), (II) or (III)) and one or more excipient selected from neutral lipids, charged lipids, steroids and polymer conjugated lipids (e.g., a pegylated lipid such as a pegylated lipid of structure (IV), such as compound IVa). In some embodiments, the at least one agent is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the lipid nanoparticles have a mean diameter of about 83 nm. In some embodiments, the lipid nanoparticles have a mean diameter of about 102 nm. In some embodiments, the lipid nanoparticles have a mean diameter of about 103 nm. In some embodiments, the lipid nanoparticles are substantially non-toxic. In certain embodiments, the at least one agent, when present in the lipid nanoparticles, is resistant in aqueous solution to degradation by intra- or intercellular enzymes.

The LNP may comprise any lipid capable of forming a particle to which the at least one agent is attached, or in which the at least one agent is encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

In some embodiments, the LNP comprises one or more cationic lipids, and one or more stabilizing lipids. Stabilizing lipids include neutral lipids and pegylated lipids.

In some embodiments, the LNP comprises a cationic lipid. As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In certain embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy) propyl)-N,N,N-trimethylammonium chloride (DOTMA); N,N-distearyl-N,N-dimethylammonium bromide (DDAB); N-(2,3-dioleoyloxy) propyl)-N,N,N-trimethylammonium chloride (DOTAP); 3-(N—(N′,N′-dimethylaminoethane)-carbamoyl) cholesterol (DC-Chol), N-(1-(2,3-dioleoyloxy) propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy) propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy) propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), N,N-dimethyl-2,3-bis(((9Z,12Z, 15Z)-octadeca-9,12,15-trien-1-yl)oxy) propan-1-amine (DLenDMA).

In some embodiments, the cationic lipid is an amino lipid. Suitable amino lipids useful in the invention include those described in WO 2012/016184, incorporated herein by reference in its entirety. Representative amino lipids include, but are not limited to, 1,2-dilinoleyoxy-3-(dimethylamino) acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino) propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino) ethoxypropane (DLin-EG-DMA), and 2,2-dilinoleyl-4-dimethylaminomethyl-[1,3]-dioxolane (DLin-K-DMA).

Suitable amino lipids include those having the formula:

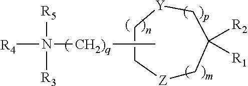

wherein R₁ and R₂ are either the same or different and independently optionally substituted C₁₀-C₂₄ alkyl, optionally substituted C₁₀-C₂₄ alkenyl, optionally substituted C₁₀-C₂₄ alkynyl, or optionally substituted C₁₀-C₂₄ acyl;
R₃ and R₄ are either the same or different and independently optionally substituted C₁-C₆ alkyl, optionally substituted C₂-C₆ alkenyl, or optionally substituted C₂-C₆ alkynyl or R₃ and R₄ may join to form an optionally substituted heterocyclic ring of 4 to 6 carbon atoms and 1 or 2 heteroatoms chosen from nitrogen and oxygen;
R₅ is either absent or present and when present is hydrogen or C₁-C₆ alkyl;
m, n, and p are either the same or different and independently either 0 or 1 with the proviso that m, n, and p are not simultaneously 0;
q is 0, 1, 2, 3, or 4; and
Y and Z are either the same or different and independently O, S, or NH.

In some embodiments, R₁ and R₂ are each linoleyl, and the amino lipid is a dilinoleyl amino lipid. In some embodiments, the amino lipid is a dilinoleyl amino lipid.

A representative useful dilinoleyl amino lipid has the formula:

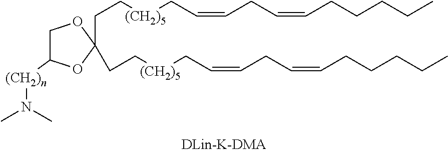

wherein n is 0, 1, 2, 3, or 4.

In some embodiments, the cationic lipid is a DLin-K-DMA. In some embodiments, the cationic lipid is DLin-KC2-DMA (DLin-K-DMA above, wherein n is 2).

In some embodiments, the cationic lipid component of the LNPs has the structure of Formula (I):

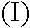

or a pharmaceutically acceptable salt, tautomer, prodrug or stereoisomer thereof, wherein:
L¹ and L² are each independently —O(C═O)—, —(C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl;
R⁸ and R⁹ are each independently C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24;
b and c are each independently an integer from 1 to 24; and
e is 1 or 2.

In certain embodiments of Formula (I), at least one of R^{1a}, R^{2a}, R^{3a} or R^{4a} is C₁-C₁₂ alkyl, or at least one of L¹ or L² is —O(C═O)— or —(C═O)O—. In other embodiments, R^{1a} and R^{1b} are not isopropyl when a is 6 or n-butyl when a is 8.

In still further embodiments of Formula (I), at least one of R^{1a}, R^{2a}, R^{3a} or R^{4a} is C₁-C₁₂ alkyl, or at least one of L¹ or L² is —O(C═O)— or —(C═O)O—; and
R^{1a} and R^{1b} are not isopropyl when a is 6 or n-butyl when a is 8.

In other embodiments of Formula (I), R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;

In certain embodiments of Formula (I), any one of L¹ or L² may be —O(C═O)— or a carbon-carbon double bond. L¹ and L² may each be —O(C═O)— or may each be a carbon-carbon double bond.

In some embodiments of Formula (I), one of L¹ or L² is —O(C═O)—. In other embodiments, both L¹ and L² are —O(C═O)—.

In some embodiments of Formula (I), one of L¹ or L² is —(C═O)O—. In other embodiments, both L¹ and L² are —(C═O)O—.

In some other embodiments of Formula (I), one of L¹ or L² is a carbon-carbon double bond. In other embodiments, both L¹ and L² are a carbon-carbon double bond.

In still other embodiments of Formula (I), one of L¹ or L² is —O(C═O)— and the other of L¹ or L² is —(C═O)O—. In more embodiments, one of L¹ or L² is —O(C═O)— and the other of L¹ or L² is a carbon-carbon double bond. In yet more embodiments, one of L¹ or L² is —(C═O)O— and the other of L¹ or L² is a carbon-carbon double bond.

It is understood that “carbon-carbon” double bond, as used throughout the specification, refers to one of the following structures:

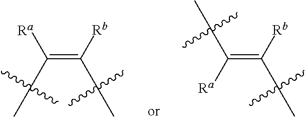

wherein R^{a} and R^{b} are, at each occurrence, independently H or a substituent. For example, in some embodiments R^{a} and R^{b} are, at each occurrence, independently H, C₁-C₁₂ alkyl or cycloalkyl, for example H or C₁-C₁₂ alkyl.

In other embodiments, the lipid compounds of Formula (I) have the following structure (Ia):

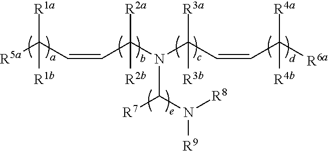

In other embodiments, the lipid compounds of Formula (I) have the following structure (Ib):

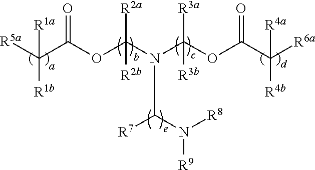

In yet other embodiments, the lipid compounds of Formula (I) have the following structure (Ic):

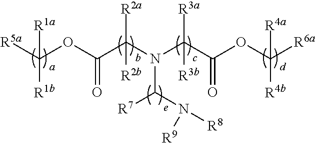

In certain embodiments of the lipid compound of Formula (I), a, b, c and d are each independently an integer from 2 to 12 or an integer from 4 to 12. In other embodiments, a, b, c and d are each independently an integer from 8 to 12 or 5 to 9. In some certain embodiments, a is 0. In some embodiments, a is 1. In other embodiments, a is 2. In more embodiments, a is 3. In yet other embodiments, a is 4. In some embodiments, a is 5. In other embodiments, a is 6. In more embodiments, a is 7. In yet other embodiments, a is 8. In some embodiments, a is 9. In other embodiments, a is 10. In more embodiments, a is 11. In yet other embodiments, a is 12. In some embodiments, a is 13. In other embodiments, a is 14. In more embodiments, a is 15. In yet other embodiments, a is 16.

In some other embodiments of Formula (I), b is 1. In other embodiments, b is 2. In more embodiments, b is 3. In yet other embodiments, b is 4. In some embodiments, b is 5. In other embodiments, b is 6. In more embodiments, b is 7. In yet other embodiments, b is 8. In some embodiments, b is 9. In other embodiments, b is 10. In more embodiments, b is 11. In yet other embodiments, b is 12. In some embodiments, b is 13. In other embodiments, b is 14. In more embodiments, b is 15. In yet other embodiments, b is 16.

In some more embodiments of Formula (I), c is 1. In other embodiments, c is 2. In more embodiments, c is 3. In yet other embodiments, c is 4. In some embodiments, c is 5. In other embodiments, c is 6. In more embodiments, c is 7. In yet other embodiments, c is 8. In some embodiments, c is 9. In other embodiments, c is 10. In more embodiments, c is 11. In yet other embodiments, c is 12. In some embodiments, c is 13. In other embodiments, c is 14. In more embodiments, c is 15. In yet other embodiments, c is 16.

In some certain other embodiments of Formula (I), d is 0. In some embodiments, dis 1. In other embodiments, d is 2. In more embodiments, d is 3. In yet other embodiments, dis 4. In some embodiments, d is 5. In other embodiments, d is 6. In more embodiments, dis 7. In yet other embodiments, d is 8. In some embodiments, d is 9. In other embodiments, d is 10. In more embodiments, d is 11. In yet other embodiments, dis 12. In some embodiments, dis 13. In other embodiments, dis 14. In more embodiments, dis 15. In yet other embodiments, dis 16.

In some other various embodiments of Formula (I), a and d are the same. In some other embodiments, b and c are the same. In some other specific embodiments, a and d are the same and b and c are the same.

The sum of a and b and the sum of c and d in Formula (I) are factors which may be varied to obtain a lipid of Formula (I) having the desired properties. In some embodiments, a and b are chosen such that their sum is an integer ranging from 14 to 24. In other embodiments, c and d are chosen such that their sum is an integer ranging from 14 to 24. In further embodiment, the sum of a and b and the sum of c and d are the same. For example, in some embodiments the sum of a and b and the sum of c and d are both the same integer which may range from 14 to 24. In still more embodiments, a. b, c and d are selected such the sum of a and b and the sum of c and dis 12 or greater.

In some embodiments of Formula (I), e is 1. In other embodiments, e is 2.

The substituents at R^{1a}, R^{2a}, R^{3a} and R^{4a} of Formula (I) are not particularly limited. In certain embodiments R^{1a}, R^{2a}, R^{3a} and R^{4a} are H at each occurrence. In certain other embodiments at least one of R^{1a}, R^{2a}, R^{3a} and R^{4a} is C₁-C₁₂ alkyl. In certain other embodiments at least one of R^{1a}, R^{2a}, R^{3a} and R^{4a} is C₁-C₈ alkyl. In certain other embodiments at least one of R^{1a}, R^{2a}, R^{3a} and R^{4a} is C₁-C₆ alkyl. In some of the foregoing embodiments, the C₁-C₈alkyl is methyl, ethyl, n-propyl, iso-propyl, n-butyl, iso-butyl, tert-butyl, n-hexyl or n-octyl.

In certain embodiments of Formula (I), R^{1a}, R^{1b}, R^{4a} and R^{4b} are C₁-C₁₂ alkyl at each occurrence.

In further embodiments of Formula (I), at least one of R^{1b}, R^{2b}, R^{3b} and R^{4b} is H or R^{1b}, R^{2b}, R^{3b} and R^{4b} are H at each occurrence.

In certain embodiments of Formula (I), R^{1b} together with the carbon atom to which it is bound is taken together with an adjacent R^{1b} and the carbon atom to which it is bound to form a carbon-carbon double bond. In other embodiments of the foregoing R^{4b} together with the carbon atom to which it is bound is taken together with an adjacent R^{4b} and the carbon atom to which it is bound to form a carbon-carbon double bond.

The substituents at R⁵ and R⁶ of Formula (I) are not particularly limited in the foregoing embodiments. In certain embodiments one or both of R⁵ or R⁶ is methyl. In certain other embodiments one or both of R⁵ or R⁶ is cycloalkyl for example cyclohexyl. In these embodiments the cycloalkyl may be substituted or not substituted. In certain other embodiments the cycloalkyl is substituted with C₁-C₁₂ alkyl, for example tert-butyl.

The substituents at R⁷ are not particularly limited in the foregoing embodiments of Formula (I). In certain embodiments at least one R⁷ is H. In some other embodiments, R⁷ is H at each occurrence. In certain other embodiments R⁷ is C₁-C₁₂ alkyl.

In certain other of the foregoing embodiments of Formula (I), one of R⁸ or R⁹ is methyl. In other embodiments, both R⁸ and R⁹ are methyl.

In some different embodiments of Formula (I), R⁸ and R⁹, together with the nitrogen atom to which they are attached, form a 5, 6 or 7-membered heterocyclic 5 ring. In some embodiments of the foregoing, R⁸ and R⁹, together with the nitrogen atom to which they are attached, form a 5-membered heterocyclic ring, for example a pyrrolidinyl ring.

In various different embodiments, exemplary lipid of Formula (I) can include

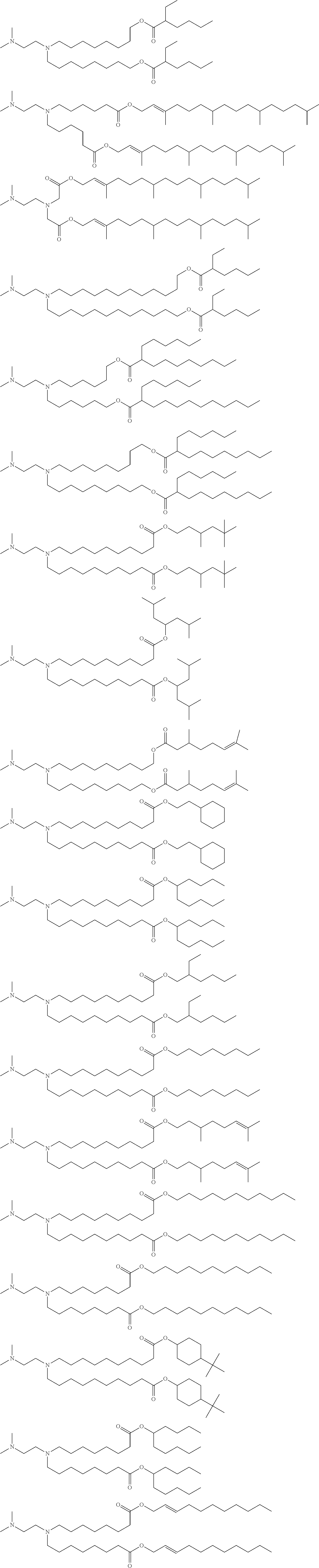

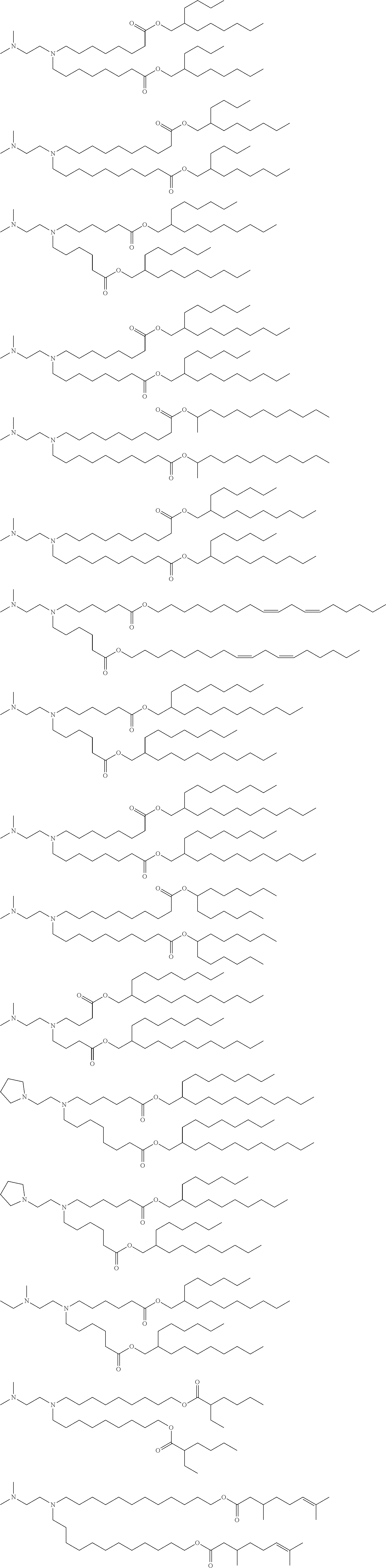

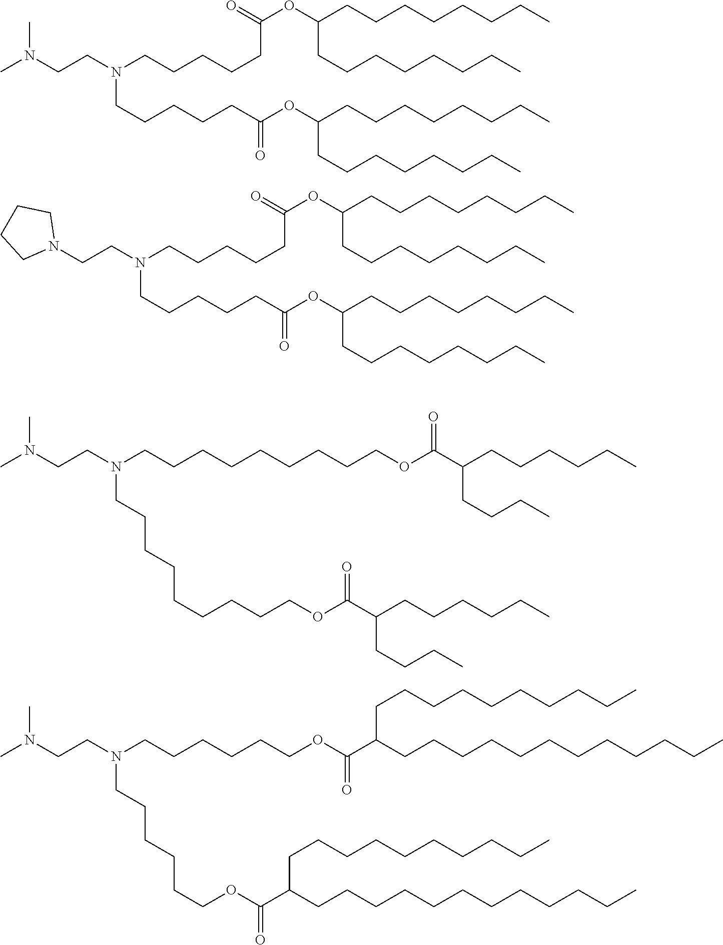

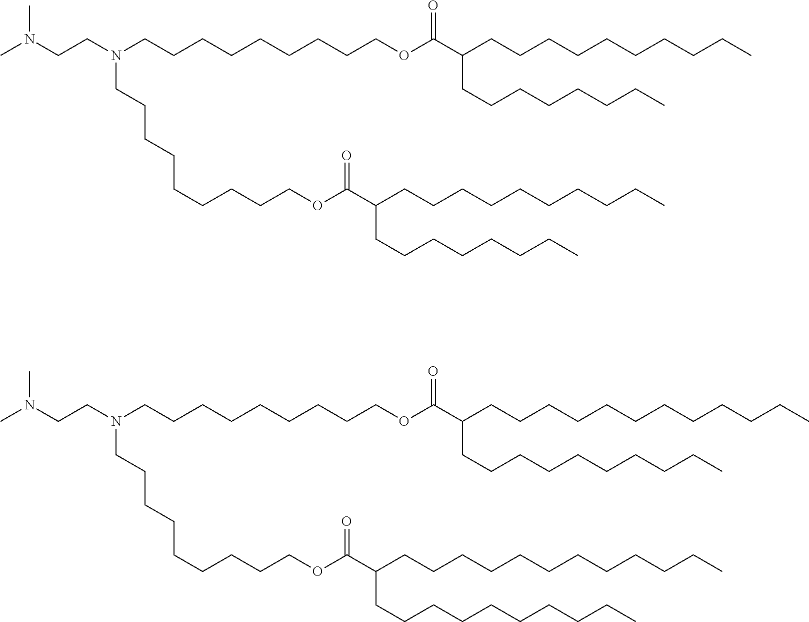

In some embodiments, the LNPs comprise a lipid of Formula (I), at least one agent, and one or more excipients selected from neutral lipids, steroids and pegylated lipids. In some embodiments the lipid of Formula (I) is compound I-5. In some embodiments the lipid of Formula (I) is compound I-6.

In some other embodiments, the cationic lipid component of the LNPs has the structure of Formula (II):

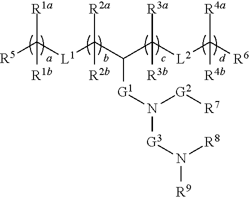

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}, —OC(═O)NR^{a}—, —NR^{a}C(═O)O—, or a direct bond;
G¹ is C₁-C₂ alkylene, —(C═O)—, —O(C═O)—, —SC(═O)—, —NR^{a}C(═O)— or a direct bond;
G² is —C(═O)—, —(C═O)O—, —C(═O)S—, —C(═O)NR^{a} or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either: (a) H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and
x is 0, 1 or 2.

In some embodiments of Formula (II), L¹ and L² are each independently —O(C═O)—, —(C═O)O— or a direct bond. In other embodiments, G¹ and G² are each independently —(C—O)— or a direct bond. In some different embodiments, L¹ and L² are each independently —O(C—O)—, —(C═O)O— or a direct bond; and G¹ and G² are each independently-(C═O)— or a direct bond.

In some different embodiments of Formula (II), L¹ and L² are each independently-C(═O)—, —O—, —S(O)ₓ—, —S—S—, —C(═O)S—, —SC(═O)—, —NR^{a}—, —NRC(═O)—, —C(═O)NR^{a}—, —NR^{a}C(═O)NR^{a}, —OC(═O)NR^{a}—, —NR^{a}C(═O)O—, —NR^{a}S(O)ₓNR^{a}—, —NR^{a}S(O)ₓ— or —S(O)ₓNR^{a}—.

In other of the foregoing embodiments of Formula (II), the lipid compound has one of the following structures (IIA) or (IIB):

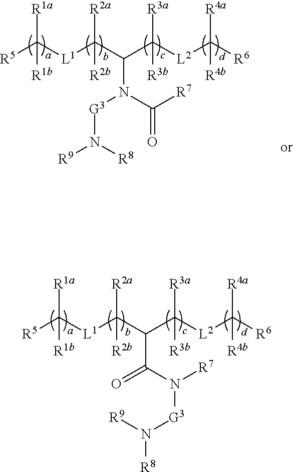

In some embodiments of Formula (II), the lipid compound has structure (IIA). In other embodiments, the lipid compound has structure (IIB).

In any of the foregoing embodiments of Formula (II), one of L¹ or L² is —O(C═O)—. For example, in some embodiments each of L¹ and L² are —O(C—O)—.

In some different embodiments of Formula (II), one of L¹ or L² is —(C═O)O—. For example, in some embodiments each of L¹ and L² is —(C═O)O—.

In different embodiments of Formula (II), one of L¹ or L² is a direct bond. As used herein, a “direct bond” means the group (e.g., L¹ or L²) is absent. For example, in some embodiments each of L¹ and L² is a direct bond.

In other different embodiments of Formula (II), for at least one occurrence of R^{1a} and R^{1b}, R^{1a} is H or C₁-C₁₂ alkyl, and R^{1b} together with the carbon atom to which it is bound is taken together with an adjacent R^{1b} and the carbon atom to which it is bound to form a carbon-carbon double bond.

In still other different embodiments of Formula (II), for at least one occurrence of R^{4a} and R^{4b}, R^{4a} is H or C₁-C₁₂ alkyl, and R^{4b} together with the carbon atom to which it is bound is taken together with an adjacent R^{4b} and the carbon atom to which it is bound to form a carbon-carbon double bond.

In more embodiments of Formula (II), for at least one occurrence of R^{2a} and R^{2b}, R^{2a} is H or C₁-C₁₂ alkyl, and R^{2b} together with the carbon atom to which it is bound is taken together with an adjacent R^{2b} and the carbon atom to which it is bound to form a carbon-carbon double bond.

In other different embodiments of Formula (II), for at least one occurrence of R^{3a} and R^{3b}, R^{3a} is H or C₁-C₁₂ alkyl, and R^{3b} together with the carbon atom to which it is bound is taken together with an adjacent R^{3b} and the carbon atom to which it is bound to form a carbon-carbon double bond.

In various other embodiments of Formula (II), the lipid compound has one of the following structures (IIC) or (IID):

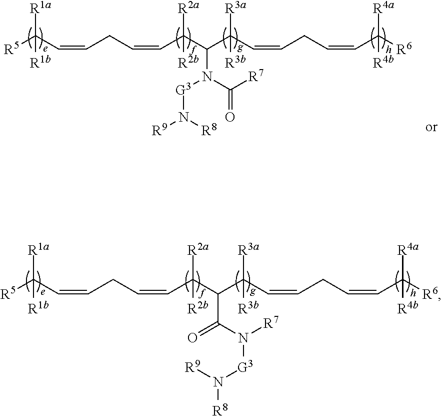

wherein e, f, g and h are each independently an integer from 1 to 12.

In some embodiments of Formula (II), the lipid compound has structure (IIC). In other embodiments, the lipid compound has structure (IID).

In various embodiments of structures (IIC) or (IID), e, f, g and h are each independently an integer from 4 to 10.

In certain embodiments of Formula (II), a, b, c and d are each independently an integer from 2 to 12 or an integer from 4 to 12. In other embodiments, a, b, c and d are each independently an integer from 8 to 12 or 5 to 9. In some certain embodiments, a is 0. In some embodiments, a is 1. In other embodiments, a is 2. In more embodiments, a is 3. In yet other embodiments, a is 4. In some embodiments, a is 5. In other embodiments, a is 6. In more embodiments, a is 7. In yet other embodiments, a is 8. In some embodiments, a is 9. In other embodiments, a is 10. In more embodiments, a is 11. In yet other embodiments, a is 12. In some embodiments, a is 13. In other embodiments, a is 14. In more embodiments, a is 15. In yet other embodiments, a is 16.

In some embodiments of Formula (II), b is 1. In other embodiments, b is 2. In more embodiments, b is 3. In yet other embodiments, b is 4. In some embodiments, b is 5. In other embodiments, b is 6. In more embodiments, b is 7. In yet other embodiments, b is 8. In some embodiments, b is 9. In other embodiments, b is 10. In more embodiments, b is 11. In yet other embodiments, b is 12. In some embodiments, b is 13. In other embodiments, b is 14. In more embodiments, b is 15. In yet other embodiments, b is 16.

In some embodiments of Formula (II), c is 1. In other embodiments, c is 2. In more embodiments, c is 3. In yet other embodiments, c is 4. In some embodiments, c is 5. In other embodiments, c is 6. In more embodiments, c is 7. In yet other embodiments, c is 8. In some embodiments, c is 9. In other embodiments, c is 10. In more embodiments, c is 11. In yet other embodiments, c is 12. In some embodiments, c is 13. In other embodiments, c is 14. In more embodiments, c is 15. In yet other embodiments, c is 16.

In some certain embodiments of Formula (II), d is 0. In some embodiments, d is 1. In other embodiments, dis 2. In more embodiments, d is 3. In yet other embodiments, dis 4. In some embodiments, d is 5. In other embodiments, d is 6. In more embodiments, dis 7. In yet other embodiments, d is 8. In some embodiments, d is 9. In other embodiments, dis 10. In more embodiments, d is 11. In yet other embodiments, dis 12. In some embodiments, dis 13. In other embodiments, dis 14. In more embodiments, dis 15. In yet other embodiments, dis 16.

In some embodiments of Formula (II), e is 1. In other embodiments, e is 2. In more embodiments, e is 3. In yet other embodiments, e is 4. In some embodiments, e is 5. In other embodiments, e is 6. In more embodiments, e is 7. In yet other embodiments, e is 8. In some embodiments, e is 9. In other embodiments, e is 10. In more embodiments, e is 11. In yet other embodiments, e is 12.

In some embodiments of Formula (II), fis 1. In other embodiments, fis 2. In more embodiments, fis 3. In yet other embodiments, fis 4. In some embodiments, fis 5. In other embodiments, fis 6. In more embodiments, fis 7. In yet other embodiments, f is 8. In some embodiments, fis 9. In other embodiments, fis 10. In more embodiments, f is 11. In yet other embodiments, fis 12.

In some embodiments of Formula (II), g is 1. In other embodiments, g is 2. In more embodiments, g is 3. In yet other embodiments, g is 4. In some embodiments, g is 5. In other embodiments, g is 6. In more embodiments, g is 7. In yet other embodiments, g is 8. In some embodiments, g is 9. In other embodiments, g is 10. In more embodiments, g is 11. In yet other embodiments, g is 12.

In some embodiments of Formula (II), h is 1. In other embodiments, e is 2. In more embodiments, h is 3. In yet other embodiments, h is 4. In some embodiments, e is 5. In other embodiments, h is 6. In more embodiments, h is 7. In yet other embodiments, h is 8. In some embodiments, h is 9. In other embodiments, h is 10. In more embodiments, h is 11. In yet other embodiments, h is 12.

In some other various embodiments of Formula (II), a and d are the same. In some other embodiments, b and c are the same. In some other specific embodiments and a and d are the same and b and c are the same.

The sum of a and b and the sum of c and d of Formula (II) are factors which may be varied to obtain a lipid having the desired properties. In some embodiments, a and b are chosen such that their sum is an integer ranging from 14 to 24. In other embodiments, c and d are chosen such that their sum is an integer ranging from 14 to 24. In further embodiment, the sum of a and b and the sum of c and d are the same. For example, in some embodiments the sum of a and b and the sum of c and d are both the same integer which may range from 14 to 24. In still more embodiments, a. b, c and d are selected such that the sum of a and b and the sum of c and dis 12 or greater.

The substituents at R^{1a}, R^{2a}, R^{3a} and R^{4a} of Formula (II) are not particularly limited. In some embodiments, at least one of R^{1a}, R^{2a}, R^{3a} and R^{4a} is H. In certain embodiments R^{1a}, R^{2a}, R^{3a} and R^{4a} are H at each occurrence. In certain other embodiments at least one of R^{1a}, R^{2a}, R^{3a} and R^{4a} is C₁-C₁₂ alkyl. In certain other embodiments at least one of R^{1a}, R^{2a}, R^{3a} and R^{4a} is C₁-C₈alkyl. In certain other embodiments at least one of R^{1a}, R^{2a}, R^{3a} and R^{4a} is C₁-C₆ alkyl. In some of the foregoing embodiments, the C₁-C₈alkyl is methyl, ethyl, n-propyl, iso-propyl, n-butyl, iso-butyl, tert-butyl, n-hexyl or n-octyl.

In certain embodiments of Formula (II), R^{1a}, R^{1b}, R^{4a} and R^{4b} are C₁-C₁₂ alkyl at each occurrence.

In further embodiments of Formula (II), at least one of R^{1b}, R^{2b}, R^{3b} and R^{4b} is H or R^{1b}, R^{2b}, R^{3b} and R^{4b} are H at each occurrence.

In certain embodiments of Formula (II), R^{1b} together with the carbon atom to which it is bound is taken together with an adjacent R^{1b} and the carbon atom to which it is bound to form a carbon-carbon double bond. In other embodiments of the foregoing R^{4b} together with the carbon atom to which it is bound is taken together with an adjacent R^{4b} and the carbon atom to which it is bound to form a carbon-carbon double bond.

The substituents at R⁵ and R⁶ of Formula (II) are not particularly limited in the foregoing embodiments. In certain embodiments one of R⁵ or R⁶ is methyl. In other embodiments each of R⁵ or R⁶ is methyl.

The substituents at R⁷ of Formula (II) are not particularly limited in the foregoing embodiments. In certain embodiments R⁷ is C₆-C₁₆ alkyl. In some other embodiments, R⁷ is C₆-C₉ alkyl. In some of these embodiments, R⁷ is substituted with —(C═O)OR^{b}, —O(C═O)R^{b}, —C(═O)R^{b}, —OR^{b}, —S(O)ₓR^{b}, —S—SR^{b}, —C(═O) SR^{b}, —SC(═O)R^{b}, —NR^{a}R^{b}, —NR^{a}C(═O)R^{b}, —C(═O)NR^{a}R^{b}, —NR^{a}C(═O)NR^{a}R^{b}, —OC(═O)NR^{a}R^{b}, —NR^{a}C(═O)OR^{b}, —NR^{a}S(O)ₓNR^{a}R^{b}, —NR^{a}S(O)ₓR^{b} or —S(O)ₓNR^{a}R^{b}, wherein: R^{a} is H or C₁-C₁₂ alkyl; R^{b} is C₁-C₁₅ alkyl; and x is 0, 1 or 2. For example, in some embodiments R⁷ is substituted with —(C═O)OR^{b} or —O(C—O)R^{b}.

In various of the foregoing embodiments of Formula (II), R^{b} is branched C₁-C₁₅ alkyl. For example, in some embodiments R^{b} has one of the following structures:

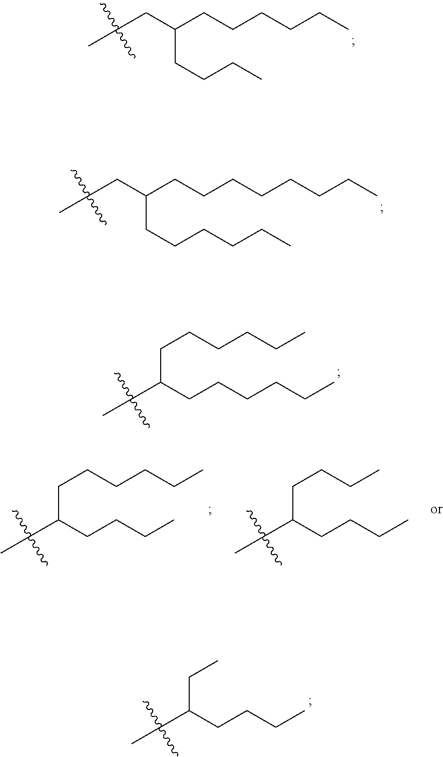

In certain other of the foregoing embodiments of Formula (II), one of R⁸ or R⁹ is methyl. In other embodiments, both R⁸ and R⁹ are methyl.

In some different embodiments of Formula (II), R⁸ and R⁹, together with the nitrogen atom to which they are attached, form a 5, 6 or 7-membered heterocyclic ring. In some embodiments of the foregoing, R⁸ and R⁹, together with the nitrogen atom to which they are attached, form a 5-membered heterocyclic ring, for example a pyrrolidinyl ring. In some different embodiments of the foregoing, R⁸ and R⁹, together with the nitrogen atom to which they are attached, form a 6-membered heterocyclic ring, for example a piperazinyl ring.

In still other embodiments of the foregoing lipids of Formula (II), G³ is C₂-C₄ alkylene, for example C₃ alkylene.

In various different embodiments, the lipid compound has one of the following structures:

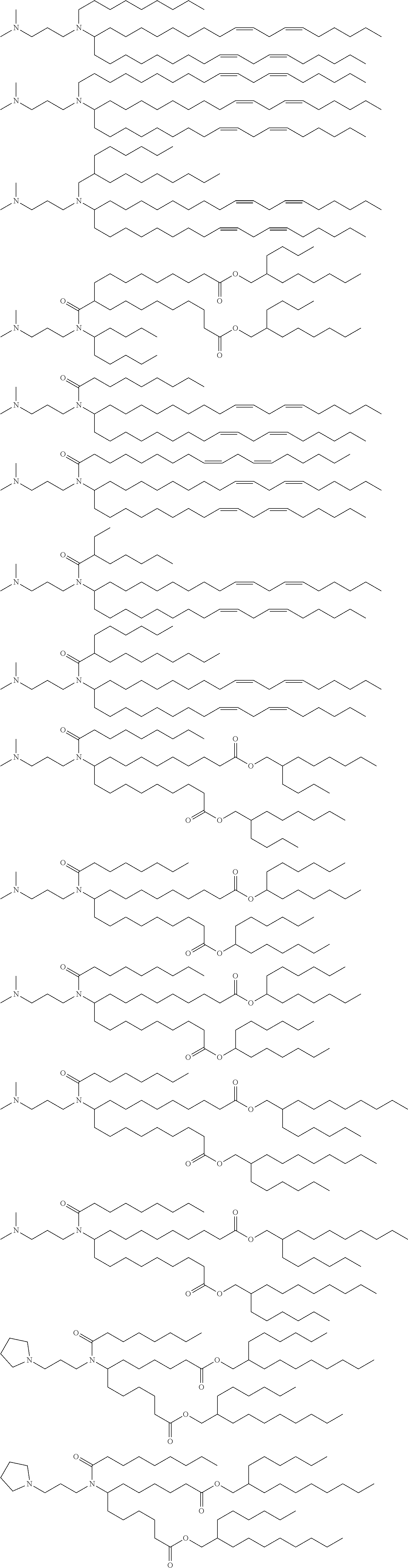

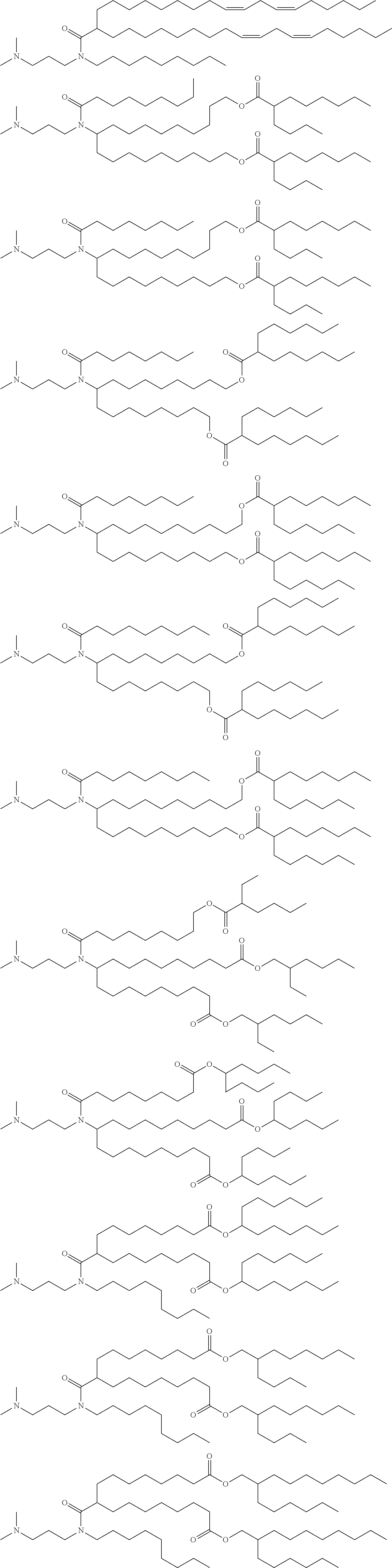

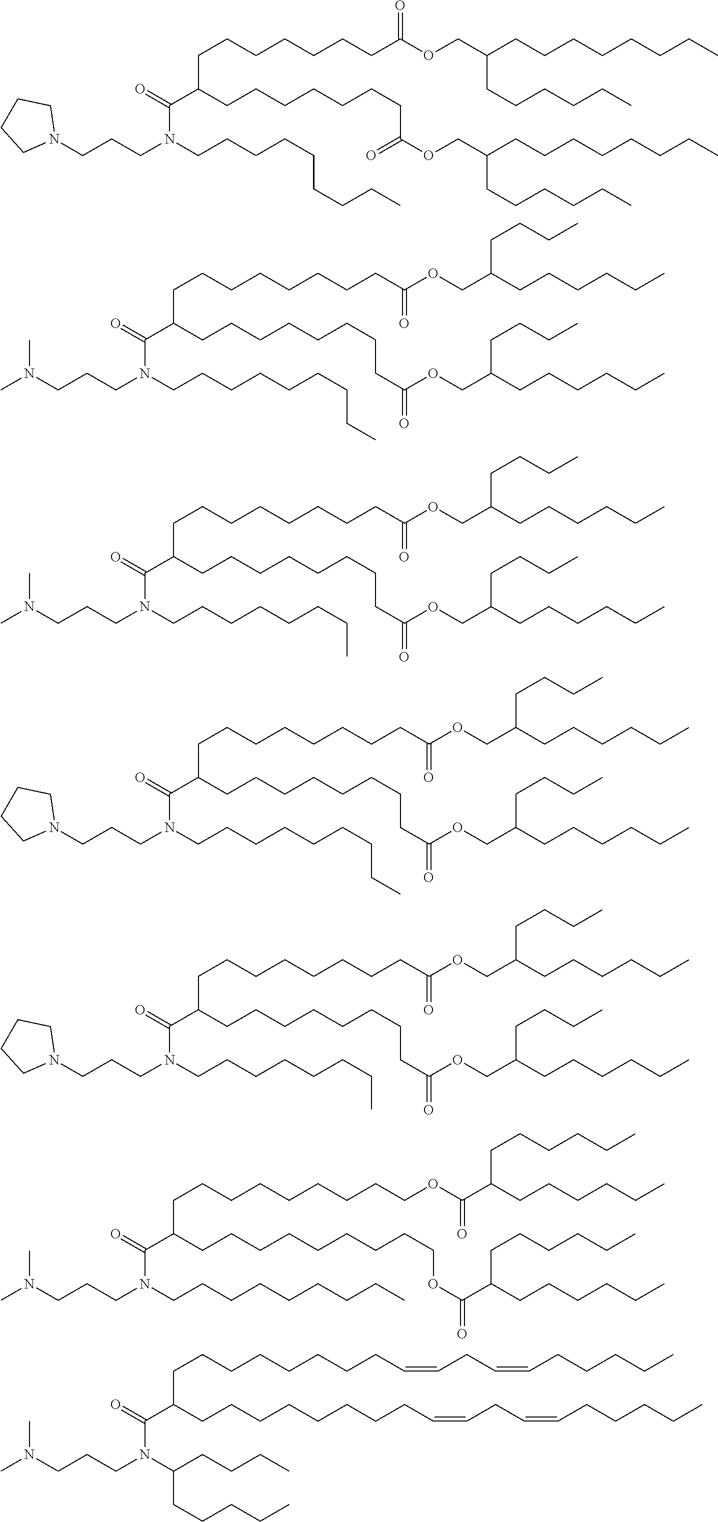

In some embodiments, the LNPs comprise a lipid of Formula (II), at least one agent, and one or more excipient selected from neutral lipids, steroids and pegylated lipids. In some embodiments, the lipid of Formula (II) is compound II-9. In some embodiments, the lipid of Formula (II) is compound II-10. In some embodiments, the lipid of Formula (II) is compound II-11. In some embodiments, the lipid of Formula (II) is compound II-12. In some embodiments, the lipid of Formula (II) is compound II-32.

In some other embodiments, the cationic lipid component of the LNPs has the structure of Formula (III):

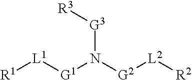

or a pharmaceutically acceptable salt, tautomer, prodrug or stereoisomer thereof, wherein:
one of L¹ or L² is —O(C—O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵° C. (═O)R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

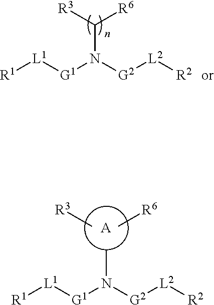

wherein:
A is a 3 to 8-membered cycloalkyl or cycloalkylene ring;
R⁶ is, at each occurrence, independently H, OH or C₁-C₂₄ alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

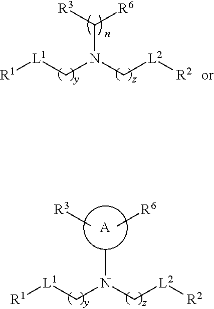

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently-(C═O)O— or —O(C═O)—. For example, in some embodiments each of L¹ and L² is —(C═O)O—.

In some different embodiments of Formula (III), the lipid has one of the following structures (IIIE) or (IIIF):

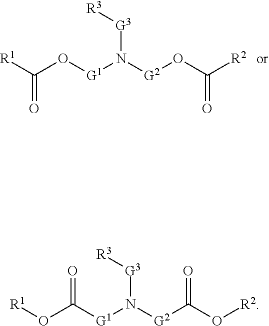

In some of the foregoing embodiments of Formula (III), the lipid has one of the following structures (IIIG), (IIIH), (IIII), or (IIIJ):

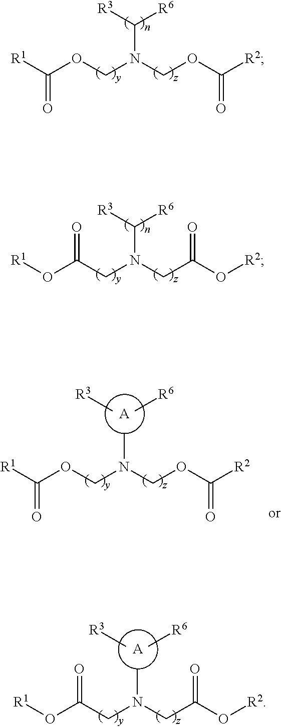

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G³ is substituted. In various different embodiments, G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

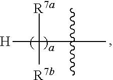

wherein:
R^{7a} and R^{7b} are, at each occurrence, independently H or C₁-C₁₂ alkyl; and
a is an integer from 2 to 12, wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

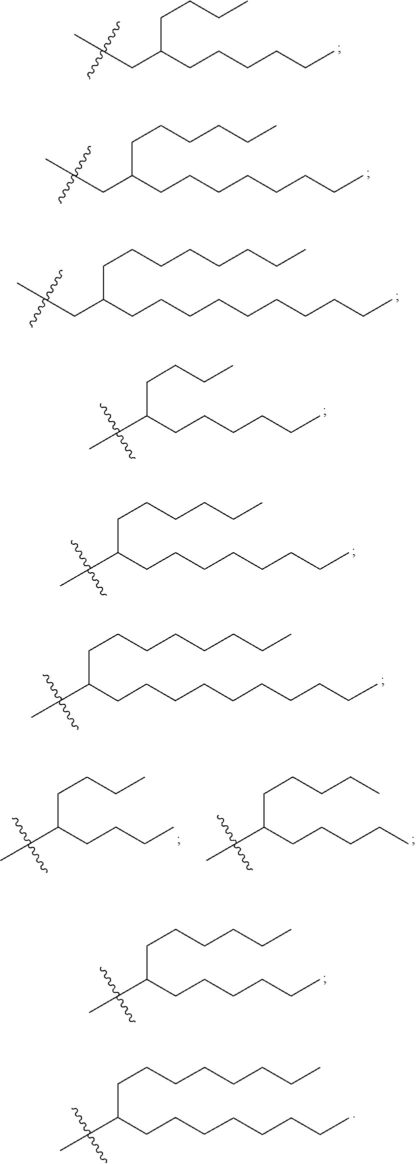

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In various different embodiments, the cationic lipid of Formula (III) has one of the following structures:

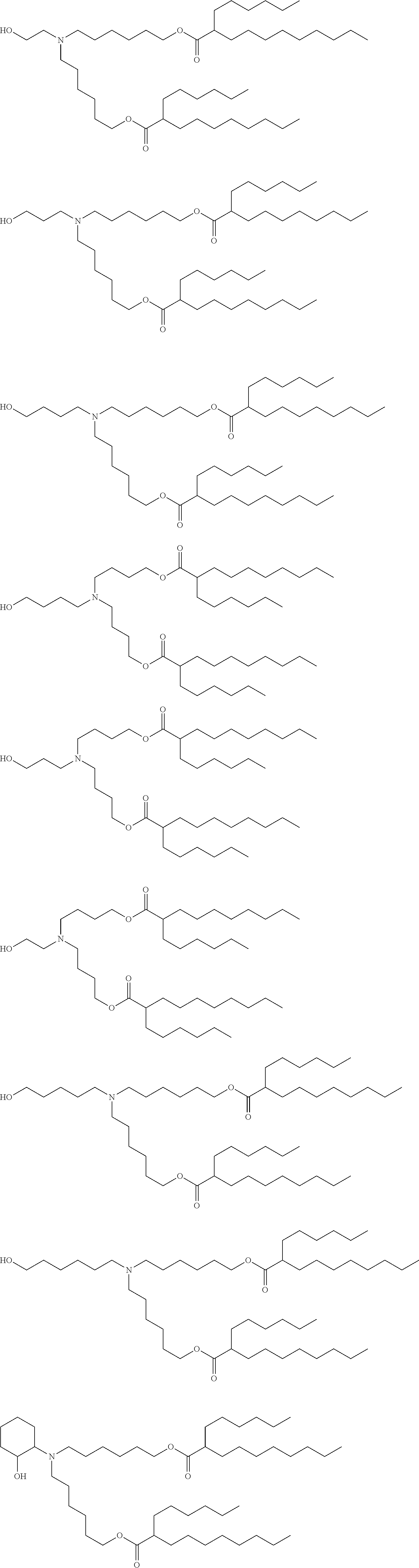

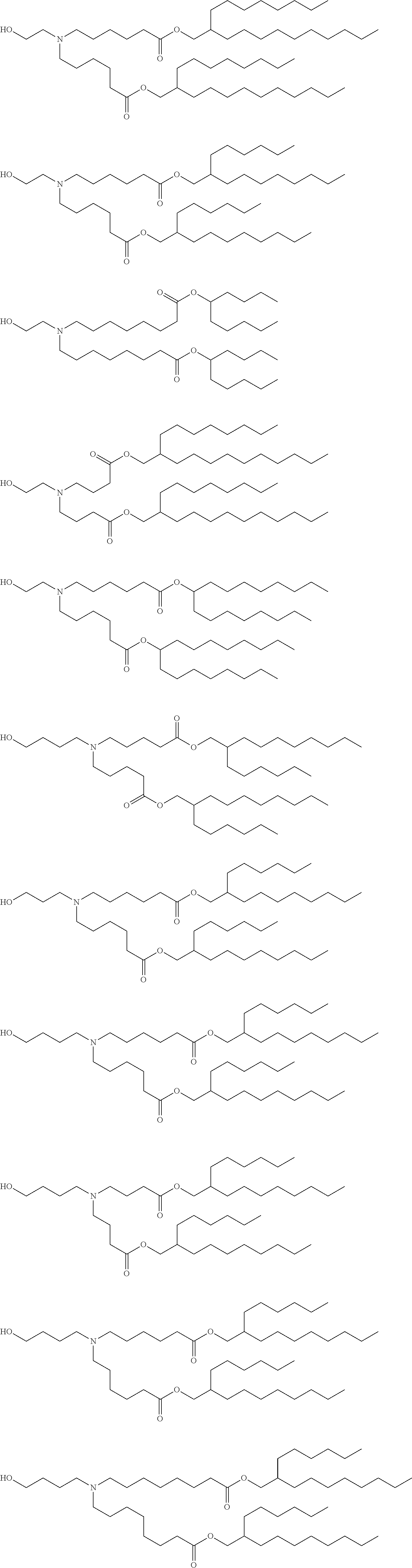

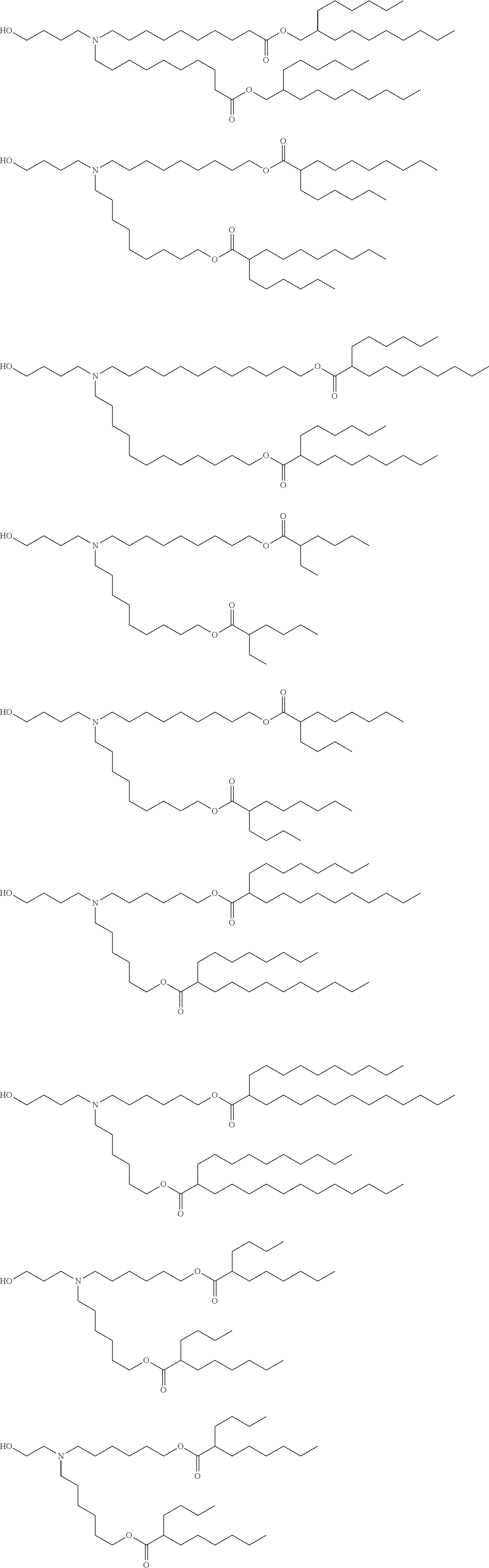

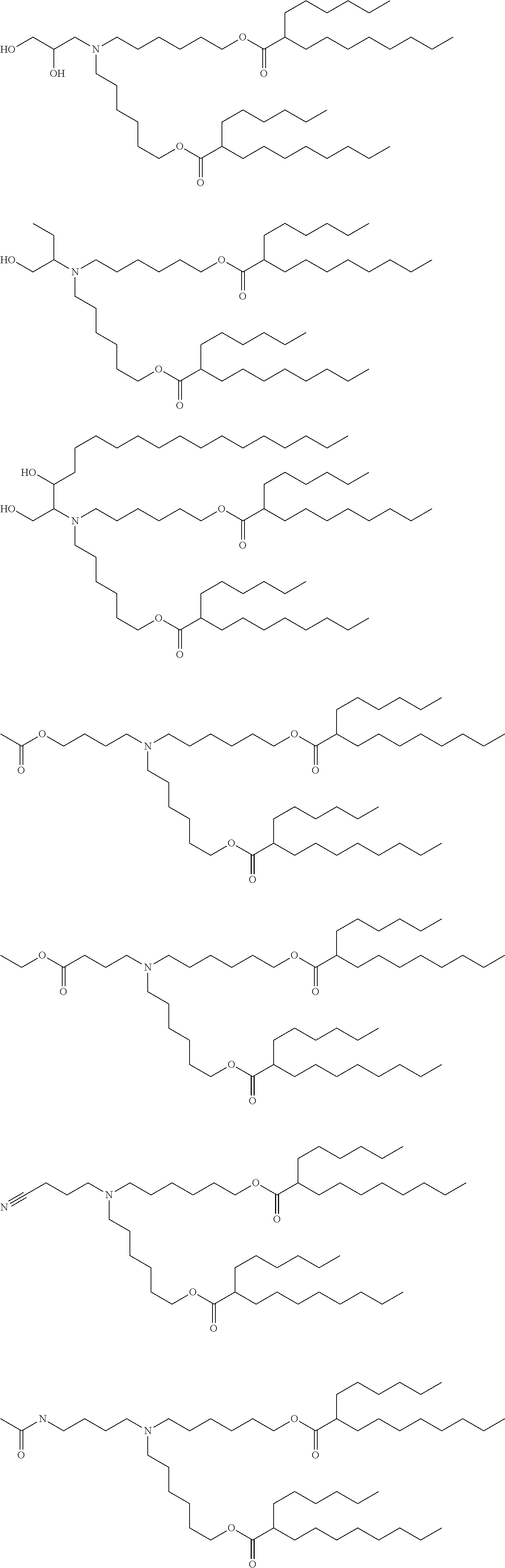

In some embodiments, the LNPs comprise a lipid of Formula (III), at least one agent, and one or more excipient selected from neutral lipids, steroids and pegylated lipids. In some embodiments, the lipid of Formula (III) is compound III-3. In some embodiments, the lipid of Formula (III) is compound III-7.

In certain embodiments, the cationic lipid is present in the LNP in an amount from about 30 to about 95 mole percent. In some embodiments, the cationic lipid is present in the LNP in an amount from about 30 to about 70 mole percent. In some embodiments, the cationic lipid is present in the LNP in an amount from about 40 to about 60 mole percent. In some embodiments, the cationic lipid is present in the LNP in an amount of about 50 mole percent. In some embodiments, the LNP comprises only cationic lipids.

In certain embodiments, the LNP comprises one or more additional lipids which stabilize the formation of particles during their formation.

Suitable stabilizing lipids include neutral lipids and anionic lipids.

The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE). In some embodiments, the neutral lipid is 1,2-distearoyl-sn-glycero-3-phosphocholine (DSPC).

In some embodiments, the LNPs comprise a neutral lipid selected from DSPC, DPPC, DMPC, DOPC, POPC, DOPE and SM. In various embodiments, the molar ratio of the cationic lipid (e.g., lipid of Formula (I)) to the neutral lipid ranges from about 2:1 to about 8:1.

In various embodiments, the LNPs further comprise a steroid or steroid analogue. A “steroid” is a compound comprising the following carbon skeleton:

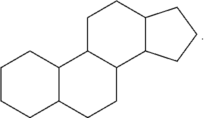 [SMILES: C1CCC2C(C1)CCC1C3CCCC3CCC21]

In certain embodiments, the steroid or steroid analogue is cholesterol. In some of these embodiments, the molar ratio of the cationic lipid (e.g., lipid of Formula (I)) to cholesterol ranges from about 2:1 to 1:1.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

In certain embodiments, the LNP comprises glycolipids (e.g., monosialoganglioside GM₁). In certain embodiments, the LNP comprises a sterol, such as cholesterol.

In some embodiments, the LNPs comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG) and the like.

In certain embodiments, the LNP comprises an additional, stabilizing-lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In some embodiments, the polyethylene glycol-lipid is N-[(methoxy poly(ethylene glycol) 2000) carbamyl]-1,2-dimyristyloxlpropyl-3-amine (PEG-c-DMA). In some embodiments, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy) propyl-1-O-(□-methoxy (polyethoxy)ethyl) butanedioate (PEG-S-DMG), a pegylated ceramide (PEG-cer), or a PEG dialkoxypropylcarbamate such as □-methoxy (polyethoxy)ethyl-N-(2,3-di(tetradecanoxy) propyl) carbamate or 2,3-di(tetradecanoxy) propyl-N-(□-methoxy (polyethoxy)ethyl) carbamate. In various embodiments, the molar ratio of the cationic lipid to the pegylated lipid ranges from about 100:1 to about 25:1.

In some embodiments, the LNPs comprise a pegylated lipid having the following structure (IV):

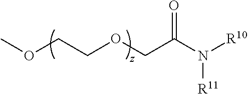

or a pharmaceutically acceptable salt, tautomer or stereoisomer thereof, wherein:
R¹⁰ and R¹¹ are each independently a straight or branched, saturated or unsaturated alkyl chain containing from 10 to 30 carbon atoms, wherein the alkyl chain is optionally interrupted by one or more ester bonds; and
z has mean value ranging from 30 to 60.

In some of the foregoing embodiments of the pegylated lipid (IV), R¹⁰ and R¹¹ are not both n-octadecyl when z is 42. In some other embodiments, R¹⁰ and R¹¹ are each independently a straight or branched, saturated or unsaturated alkyl chain containing from 10 to 18 carbon atoms. In some embodiments, R¹⁰ and R¹¹ are each independently a straight or branched, saturated or unsaturated alkyl chain containing from 12 to 16 carbon atoms. In some embodiments, R¹⁰ and R¹¹ are each independently a straight or branched, saturated or unsaturated alkyl chain containing 12 carbon atoms. In some embodiments, R¹⁰ and R¹¹ are each independently a straight or branched, saturated or unsaturated alkyl chain containing 14 carbon atoms. In other embodiments, R¹⁰ and R¹¹ are each independently a straight or branched, saturated or unsaturated alkyl chain containing 16 carbon atoms. In still more embodiments, R¹⁰ and R¹¹ are each independently a straight or branched, saturated or unsaturated alkyl chain containing 18 carbon atoms. In still other embodiments, R¹⁰ is a straight or branched, saturated or unsaturated alkyl chain containing 12 carbon atoms and R¹¹ is a straight or branched, saturated or unsaturated alkyl chain containing 14 carbon atoms.

In various embodiments, z spans a range that is selected such that the PEG portion of (II) has an average molecular weight of about 400 to about 6000 g/mol. In some embodiments, the average z is about 45.

In other embodiments, the pegylated lipid has one of the following structures:

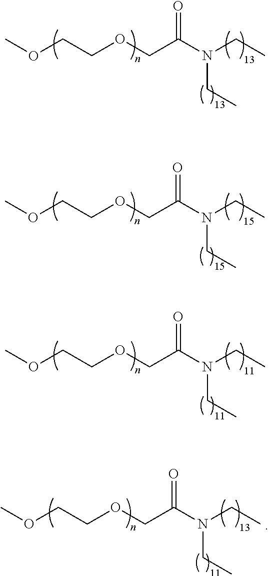

wherein n is an integer selected such that the average molecular weight of the pegylated lipid is about 2500 g/mol.

In certain embodiments, the additional lipid is present in the LNP in an amount from about 1 to about 10 mole percent. In some embodiments, the additional lipid is present in the LNP in an amount from about 1 to about 5 mole percent. In some embodiments, the additional lipid is present in the LNP in about 1 mole percent or about 1.5 mole percent.

In some embodiments, the LNPs comprise a lipid of Formula (I), a nucleoside-modified RNA, a neutral lipid, a steroid and a pegylated lipid. In some embodiments the lipid of Formula (I) is compound I-6. In different embodiments, the neutral lipid is DSPC. In other embodiments, the steroid is cholesterol. In still different embodiments, the pegylated lipid is compound IVa.

In certain embodiments, the LNP comprises one or more targeting moieties that targets the LNP to a stem cell or stem cell population. For example, In some embodiments, the targeting domain is a ligand which directs the LNP to a receptor found on a stem cell surface.

Exemplary LNPs and their manufacture are described in the art, for example in U.S. Patent Application Publication No. US20120276209, Semple et al., 2010, Nat Biotechnol., 28(2):172-176; Akinc et al., 2010, Mol Ther., 18(7): 1357-1364; Basha et al., 2011, Mol Ther, 19(12): 2186-2200; Leung et al., 2012, J Phys Chem C Nanomater Interfaces, 116(34): 18440-18450; Lee et al., 2012, Int J Cancer, 131(5): E781-90; Belliveau et al., 2012, Mol Ther nucleic Acids, 1:e37; Jayaraman et al., 2012, Angew Chem Int Ed Engl., 51(34): 8529-8533; Mui et al., 2013, Mol Ther Nucleic Acids. 2, e139; Maier et al., 2013, Mol Ther., 21(8): 1570-1578; and Tam et al., 2013, Nanomedicine, 9(5): 665-74, each of which are incorporated by reference in their entirety.

The following Reaction Schemes illustrate methods to make lipids of Formula (I), (II) or (III).

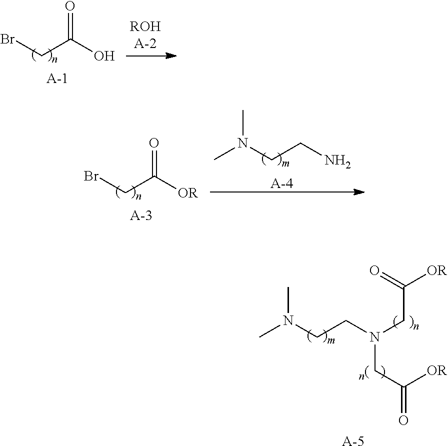

Embodiments of the lipid of Formula (I) (e.g., compound A-5) can be prepared according to General Reaction Scheme 1 (“Method A”), wherein R is a saturated or unsaturated C₁-C₂₄ alkyl or saturated or unsaturated cycloalkyl, m is 0 or 1 and n is an integer from 1 to 24. Referring to General Reaction Scheme 1, compounds of structure A-1 can be purchased from commercial sources or prepared according to methods familiar to one of ordinary skill in the art. A mixture of A-1, A-2 and DMAP is treated with DCC to give the bromide A-3. A mixture of the bromide A-3, a base (e.g., N,N-diisopropylethylamine) and the N,N-dimethyldiamine A-4 is heated at a temperature and time sufficient to produce A-5 after any necessarily workup and or purification step.

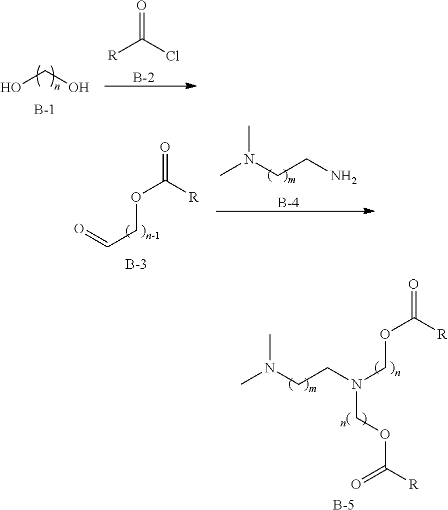

Other embodiments of the compound of Formula (I) (e.g., compound B-5) can be prepared according to General Reaction Scheme 2 (“Method B”), wherein R is a saturated or unsaturated C₁-C₂₄ alkyl or saturated or unsaturated cycloalkyl, m is 0 or 1 and n is an integer from 1 to 24. As shown in General Reaction Scheme 2, compounds of structure B-1 can be purchased from commercial sources or prepared according to methods familiar to one of ordinary skill in the art. A solution of B-1 (1 equivalent) is treated with acid chloride B-2 (1 equivalent) and a base (e.g., triethylamine). The crude product is treated with an oxidizing agent (e.g., pyridinum chlorochromate) and intermediate product B-3 is recovered. A solution of crude B-3, an acid (e.g., acetic acid), and N,N-dimethylaminoamine B-4 is then treated with a reducing agent (e.g., sodium triacetoxyborohydride) to obtain B-5 after any necessary work up and/or purification.

It should be noted that although starting materials A-1 and B-1 are depicted above as including only saturated methylene carbons, starting materials which include carbon-carbon double bonds may also be employed for preparation of compounds which include carbon-carbon double bonds.

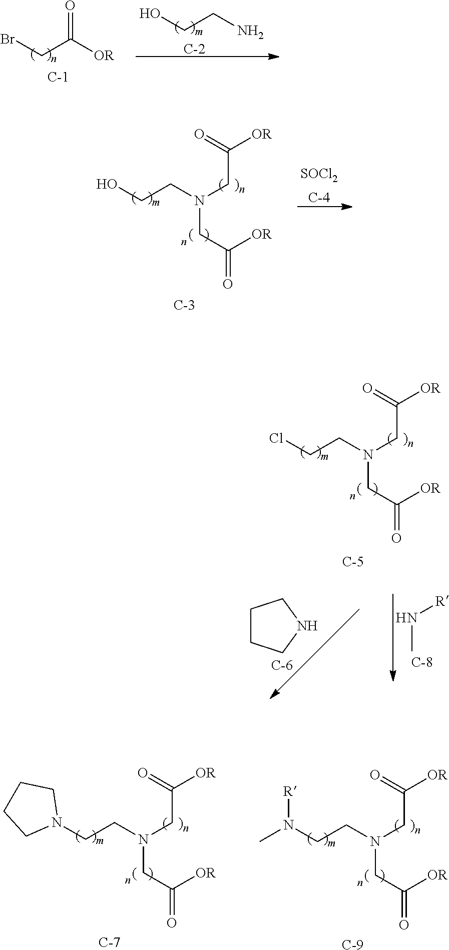

Different embodiments of the lipid of Formula (I) (e.g., compound C-7 or C₉) can be prepared according to General Reaction Scheme 3 (“Method C”), wherein R is a saturated or unsaturated C₁-C₂₄ alkyl or saturated or unsaturated cycloalkyl, m is 0 or 1 and n is an integer from 1 to 24. Referring to General Reaction Scheme 3, compounds of structure C-1 can be purchased from commercial sources or prepared according to methods familiar to one of ordinary skill in the art.

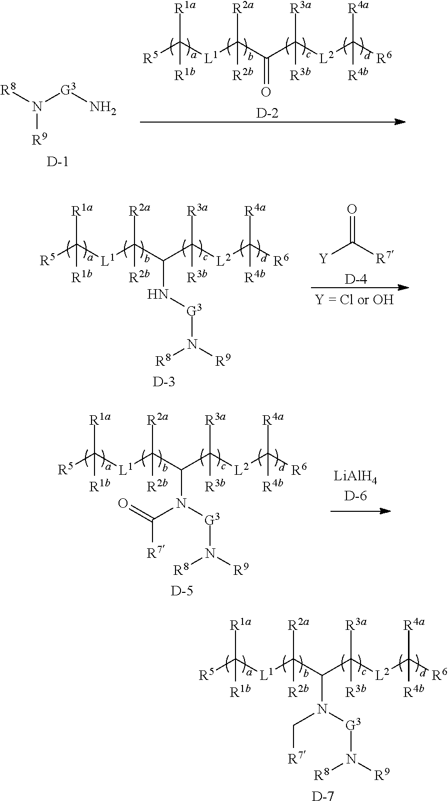

Embodiments of the compound of Formula (II) (e.g., compounds D-5 and D-7) can be prepared according to General Reaction Scheme 4 (“Method D”), wherein R^{1a}, R^{1b}, R^{2a}, R^{2b}, R^{3a}, R^{3b}, R^{4a}, R^{4b}, R⁵, R⁶, R⁸, R⁹, L¹, L², G¹, G², G³, a, b, c and d are as defined herein, and R⁷ represents R⁷ or a C₃-C₁₉ alkyl. Referring to General Reaction Scheme 1, compounds of structure D-1 and D-2 can be purchased from commercial sources or prepared according to methods familiar to one of ordinary skill in the art. A solution of D-1 and D-2 is treated with a reducing agent (e.g., sodium triacetoxyborohydride) to obtain D-3 after any necessary work up. A solution of D-3 and a base (e.g. trimethylamine, DMAP) is treated with acyl chloride D-4 (or carboxylic acid and DCC) to obtain D-5 after any necessary work up and/or purification. D-5 can be reduced with LiAlH4 D-6 to give D-7 after any necessary work up and/or purification.

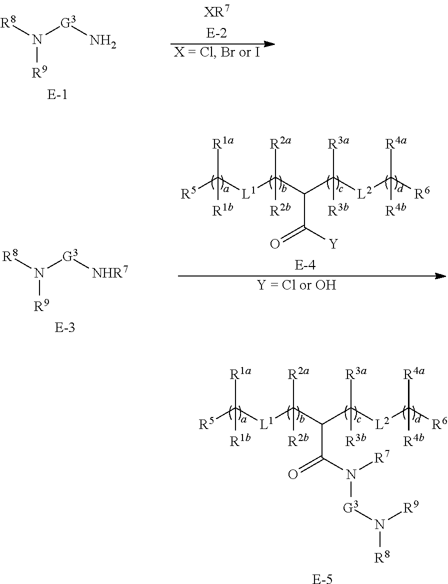

Embodiments of the lipid of Formula (II) (e.g., compound E-5) can be prepared according to General Reaction Scheme 5 (“Method E”), wherein R^{1a}, R^{1b}, R^{2a}, R^{2b}, R^{3a}, R^{3b}, R^{4a}, R^{4b}, R⁵, R⁶, R⁷, R⁸, R⁹, L¹, L², G³, a, b, c and d are as defined herein. Referring to General Reaction Scheme 2, compounds of structure E-1 and E-2 can be purchased from commercial sources or prepared according to methods familiar to one of ordinary skill in the art. A mixture of E-1 (in excess), E-2 and a base (e.g., potassium carbonate) is heated to obtain E-3 after any necessary work up. A solution of E-3 and a base (e.g. trimethylamine, DMAP) is treated with acyl chloride E-4 (or carboxylic acid and DCC) to obtain E-5 after any necessary work up and/or purification.

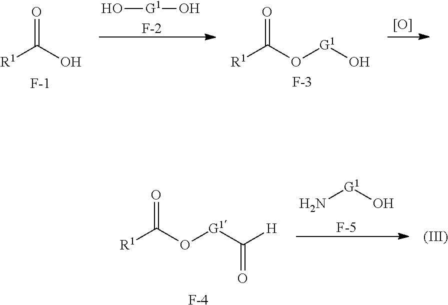

General Reaction Scheme 6 provides an exemplary method (Method F) for preparation of Lipids of Formula (III). G¹, G³, R¹ and R³ in General Reaction Scheme 6 are as defined herein for Formula (III), and G¹′ refers to a one-carbon shorter homologue of G¹. Compounds of structure F-1 are purchased or prepared according to methods known in the art. Reaction of F-1 with diol F-2 under appropriate condensation conditions (e.g., DCC) yields ester/alcohol F-3, which can then be oxidized (e.g., PCC) to aldehyde F-4. Reaction of F-4 with amine F-5 under reductive amination conditions yields a lipid of Formula (III).

It should be noted that various alternative strategies for preparation of lipids of Formula (III) are available to those of ordinary skill in the art. For example, other lipids of Formula (III) wherein L¹ and L² are other than ester can be prepared according to analogous methods using the appropriate starting material. Further, General Reaction Scheme 6 depicts preparation of a lipids of Formula (III), wherein G¹ and G² are the same; however, this is not a required aspect of the invention and modifications to the above reaction scheme are possible to yield compounds wherein G¹ and G² are different. It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include-C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

### Targeting Domain

In some embodiments, the targeting domain specifically binds to CD117. The targeting domain may comprise a nucleic acid, peptide, antibody, small molecule, organic molecule, inorganic molecule, glycan, sugar, hormone, and the like that targets the particle to a site in particular need of the therapeutic agent. In certain embodiments, the particle comprises multivalent targeting, wherein the particle comprises multiple targeting mechanisms described herein.

In some embodiments, the targeting domain may be co-polymerized with the composition comprising the delivery vehicle. In some embodiments, the targeting domain may be covalently attached to the composition comprising the delivery vehicle, such as through a chemical reaction between the targeting domain and the composition comprising the delivery vehicle. In some embodiments, the targeting domain is an additive in the delivery vehicle. Targeting domains of the instant invention include, but are not limited to, antibodies, antibody fragments, proteins, peptides, and nucleic acids.

In some embodiments, the composition comprises a targeting domain that directs the delivery vehicle to CD117. In some embodiments, the targeting domain is an affinity ligand which specifically binds to CD117.

In one aspect, the present invention relates to composition comprising a delivery vehicle conjugated to a CD117 targeting domain. In certain embodiments, the targeting domain binds to CD117 expressed on the surface of a target stem cell, thereby directing the composition to the target stem cell.

### Peptides

In some embodiments, the targeting domain of the invention comprises a peptide. In certain embodiments, the peptide targeting domain specifically binds to marker of a cell type of interest. In some embodiments, the targeting domain directs the vehicle to an endothelial cell, an immune cell, a stem cell, or another specific cell type of interest. For example, In some embodiments, the targeting domain directs the vehicle to a CD117 expressing stem cell.

The peptide of the present invention may be made using chemical methods. For example, peptides can be synthesized by solid phase techniques (Roberge J Y et al (1995) Science 269:202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the peptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one peptide to a sequence of a second peptide. Variants are defined to include peptide sequences different from the original sequence. In some embodiments, variants are different from the original sequence in less than 40% of residues per segment of interest. In some embodiments, variants are different from the original sequence in less than 25% of residues per segment of interest. In some embodiments, variants are different by less than 10% of residues per segment of interest. In some embodiments, variants are different from the original protein sequence in just a few residues per segment of interest and at the same time sufficiently homologous to the original sequence to preserve the functionality of the original sequence. The present invention includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. In some embodiments, the identity between two amino acid sequences is determined by using the BLASTP algorithm [BLAST Manual, Altschul, S., et al., NCBI NLM NIH Bethesda, Md. 20894, Altschul, S., et al., J. Mol. Biol. 215:403-410 (1990)].

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation.

### Nucleic Acids

In some embodiments, the targeting domain of the invention comprises an isolated nucleic acid, including for example a DNA oligonucleotide and a RNA oligonucleotide. In certain embodiments, the nucleic acid targeting domain specifically binds to CD117. For example, In some embodiments, the nucleic acid comprises a nucleotide sequence that specifically binds to CD117.

The nucleotide sequences of a nucleic acid targeting domain can alternatively comprise sequence variations with respect to the original nucleotide sequences, for example, substitutions, insertions and/or deletions of one or more nucleotides, with the condition that the resulting nucleic acid functions as the original and specifically binds to CD117.

In the sense used in this description, a nucleotide sequence is “substantially homologous” to any of the nucleotide sequences describe herein when its nucleotide sequence has a degree of identity with respect to the nucleotide sequence of at least 60%. In some embodiments, the degree of identity is at least 70%. In some embodiments, the degree of identity is at least 85%. In some embodiments, the degree of identity is at least 95%. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art. In some embodiments, the identity between two amino acid sequences is determined by using the BLASTN algorithm [BLAST Manual, Altschul, S., et al., NCBI NLM NIH Bethesda, Md. 20894, Altschul, S., et al., J. Mol. Biol. 215:403-410 (1990)].

### Antibodies

In some embodiments, the targeting domain of the invention comprises an antibody, or antibody fragment. In certain embodiments, the antibody targeting domain specifically binds to CD117. Such antibodies include polyclonal antibodies, monoclonal antibodies, Fab and single chain Fv (scFv) fragments thereof, bispecific antibodies, heteroconjugates, human and humanized antibodies.

The antibodies may be intact monoclonal or polyclonal antibodies, and immunologically active fragments (e.g., a Fab or (Fab) 2 fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmokinetic activity may be generated in a bacterial expression system. Single chain Fv fragments show low immunogenicity.

### Conjugation

In various embodiments of the invention, the delivery vehicle (e.g., LNP) is conjugated to the CD117 targeting domain. Exemplary methods of conjugation can include, but are not limited to, covalent bonds, electrostatic interactions, and hydrophobic (“van der Waals”) interactions. In some embodiments, the conjugation is a reversible conjugation, such that the delivery vehicle can be disassociated from the targeting domain upon exposure to certain conditions or chemical agents. In another embodiment, the conjugation is an irreversible conjugation, such that under normal conditions the delivery vehicle does not dissociate the targeting domain.

In some embodiments, the conjugation comprises a covalent bond between an activated polymer conjugated lipid and the targeting domain. The term “activated polymer conjugated lipid” refers to a molecule comprising a lipid portion and a polymer portion that has been activated via functionalization of a polymer conjugated lipid with a first coupling group. In some embodiments, the activated polymer conjugated lipid comprises a first coupling group capable of reacting with a second coupling group. In some embodiments, the activated polymer conjugated lipid is an activated pegylated lipid. In some embodiments, the first coupling group is bound to the lipid portion of the pegylated lipid. In another embodiment, the first coupling group is bound to the polyethylene glycol portion of the pegylated lipid. In some embodiments, the second functional group is covalently attached to the targeting domain.

The first coupling group and second coupling group can be any functional groups known to those of skill in the art to together form a covalent bond, for example under mild reaction conditions or physiological conditions. In some embodiments, the first coupling group or second coupling group are selected from the group consisting of maleimides, N-hydroxysuccinimide (NHS) esters, carbodiimides, hydrazide, pentafluorophenyl (PFP) esters, phosphines, hydroxymethyl phosphines, psoralen, imidoesters, pyridyl disulfide, isocyanates, vinyl sulfones, alpha-haloacetyls, aryl azides, acyl azides, alkyl azides, diazirines, benzophenone, epoxides, carbonates, anhydrides, sulfonyl chlorides, cyclooctyne, aldehydes, and sulfhydryl groups. In some embodiments, the first coupling group or second coupling group is selected from the group consisiting of free amines (—NH₂), free sulfhydryl groups (—SH), free hydroxide groups (—OH), carboxylates, hydrazides, and alkoxyamines. In some embodiments, the first coupling group is a functional group that is reactive toward sulfhydryl groups, such as maleimide, pyridyl disulfide, or a haloacetyl. In some embodiments, the first coupling group is a maleimide.

In some embodiments, the second coupling group is a sulfhydryl group. The sulfhydryl group can be installed on the targeting domain using any method known to those of skill in the art. In some embodiments, the sulfhydryl group is present on a free cysteine residue. In some embodiments, the sulfhydryl group is revealed via reduction of a disulfide on the targeting domain, such as through reaction with 2-mercaptoethylamine. In some embodiments, the sulfhydryl group is installed via a chemical reaction, such as the reaction between a free amine and 2-iminothilane or N-succinimidyl S-acetylthioacetate (SATA).

In some embodiments, the polymer conjugated lipid and the targeting domain are functionalized with groups used in “click” chemistry. Bioorthogonal “click” chemistry comprises the reaction between a functional group with a 1,3-dipole, such as an azide, a nitrile oxide, a nitrone, an isocyanide, and the link, with an alkene or an alkyne dipolarophiles. Exemplary dipolarophiles include any strained cycloalkenes and cycloalkynes known to those of skill in the art, including, but not limited to, cyclooctynes, dibenzocyclooctynes, monofluorinated cyclcooctynes, difluorinated cyclooctynes, and biarylazacyclooctynone

### Combinations

In some embodiments, the composition of the present invention comprises a combination of agents described herein (e.g., a combination of a CD117-LNP comprising a mRNA encoding a protein for genetic editing and a CD117-LNP comprising a gRNA molecule). In certain embodiments, a composition comprising a combination of agents described herein has an additive effect, wherein the overall effect of the combination is approximately equal to the sum of the effects of each individual agent. In other embodiments, a composition comprising a combination of agents described herein has a synergistic effect, wherein the overall effect of the combination is greater than the sum of the effects of each individual agent.

A composition comprising a combination of agents comprises individual agents in any suitable ratio. For example, In some embodiments, the composition comprises a 1:1 ratio of two individual agents. However, the combination is not limited to any particular ratio. Rather any ratio that is shown to be effective is encompassed.

### Therapeutic Methods

In some embodiments, the invention provides methods for genetic editing of patient HSCs. In some embodiments, the method allows for the treatment of a monogenic HSC disease or disorder without depletion/ablation of the patient's HSC population, and with minimal or no hospitalization.

Exemplary diseases or disorders that can be treated using the methods of the invention include, but are not limited to, blood monogenic disorders, genetic defects, bone marrow genetic defects, cancers, platelet disorders, immunodeficiencies, non-hematologic diseases (e.g., cystic fibrosis), metabolic disease and autoimmune diseases.

Exemplary bone marrow genetic defects include, but are not limited to, leukemia, aplastic anemia, myeloproliferative disorders, an inherited bone marrow failure syndrome (IBMFS) such as Fanconi anemia, dyskeratosis congenital, Shwachman-Diamond syndrome, Diamond-Blackfan anemia, severe congenital neutropenia, a primary immunodeficiency such as X1-SCID and Wiskott-Aldrich syndrome, an erythroid disorder such as sickle cell disease (SCD), pyruvate kinase deficiency, or a lysosomal storage diseases such as Fabry disease and Pompe disease. In some embodiments, the SCD is sickle cell anemia.

Exemplary inflammatory conditions and autoimmune diseases include, but are not limited to, rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopeni purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis-juvenile, discoid lupus, essential mixed cryoglobulinemia, fibromyalgia-fibromyositis, Grave's disease, Guillain-Barre, Hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, Wegener's granulomatosi, and other organ-specific inflammatory disorders.

To practice the methods of the invention; the skilled artisan would understand, based on the disclosure provided herein, how to formulate and administer the appropriate composition to a subject. The present invention is not limited to any particular method of administration or treatment regimen.

In some embodiments, the method comprises delivery of one or more CD117 targeted LNP delivery vehicles, comprising at least one mRNA encoding a base editor and a guide RNA for site-specific editing of a target nucleobase, wherein each delivery vehicle is conjugated to a CD117 targeting domain.

It will be appreciated by one of skill in the art, when armed with the present disclosure including the methods detailed herein, that the invention is not limited to treatment of diseases or disorders that are already established. Particularly, the disease or disorder need not have manifested to the point of detriment to the subject; indeed, the disease or disorder need not be detected in a subject before treatment is administered. That is, significant signs or symptoms of diseases or disorders do not have to occur before the present invention may provide benefit. Therefore, the present invention includes a method for preventing diseases or disorders, in that a composition, as discussed previously elsewhere herein, can be administered to a subject prior to the onset of diseases or disorders, thereby preventing diseases or disorders.

One of skill in the art, when armed with the disclosure herein, would appreciate that the prevention of a disease or disorder, encompasses administering to a subject a composition as a preventative measure against the development of, or progression of, a disease or disorder.

One of skill in the art will appreciate that the compositions of the invention can be administered singly or in any combination. Further, the compositions of the invention can be administered singly or in any combination in a temporal sense, in that they may be administered concurrently, or before, and/or after each other. One of ordinary skill in the art will appreciate, based on the disclosure provided herein, that the compositions of the invention can be used to prevent or to treat a disease or disorder, and that a composition can be used alone or in any combination with another composition to affect a therapeutic result. In various embodiments, any of the compositions of the invention described herein can be administered alone or in combination with other modulators of other molecules associated with diseases or disorders.

In some embodiments, the invention includes a method comprising administering a combination of compositions described herein. In certain embodiments, the method has an additive effect, wherein the overall effect of the administering a combination of compositions is approximately equal to the sum of the effects of administering each individual inhibitor. In other embodiments, the method has a synergistic effect, wherein the overall effect of administering a combination of compositions is greater than the sum of the effects of administering each individual composition.

The method comprises administering a combination of composition in any suitable ratio. For example, In some embodiments, the method comprises administering two individual compositions at a 1:1 ratio. However, the method is not limited to any particular ratio. Rather any ratio that is shown to be effective is encompassed.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

In addition to the active ingredient, a pharmaceutical composition of the invention may further comprise one or more additional adjuvants. Exemplary adjuvants include, but are not limited to, aluminum-based adjuvant and monophosphoryl lipid A.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In some embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers. In some embodiments, the diameter is in a range from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. In some embodiments, such powders comprise particles wherein at least 98% of the particles by weight have a diameter greater than 0.5 nanometers and at least 95% of the particles by number have a diameter less than 7 nanometers. In some embodiments, at least 95% of the particles by weight have a diameter greater than 1 nanometer and at least 90% of the particles by number have a diameter less than 6 nanometers. In some embodiments, dry powder compositions include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent. In some embodiments, the propellant comprise particles with a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In some embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: In Vivo Modification of Hematopoietic Stem Cells by Targeted Lipid Nanoparticles Encapsulating mRNA

A novel flexible methodology that can modify HSC in vivo was developed utilizing HSC-targeted lipid nanoparticles (LNP) encapsulating mRNA. This method utilizes antibodies against CD117 conjugated to LNP (CD117/LNP-mRNA). CD117 is internalized after binding of SCF, which facilitates or augments LNP internalization (Yee, N. S., et al., 1994, J Biol Chem, 269:31991-31998). Nucleoside-modified and purified mRNA is non-immunogenic, stable, extensible, and can be used to express virtually any protein of interest (Weissman, D. and Kariko, K., 2015, Mol Ther, 23:1416-1417; Kariko, K., et al., 2005, Immunity, 23:165-175; Kariko, K., et al., 2011, Nucleic Acids Res, 29:e142). Lipid nanoparticles (LNP) are thus far the most promising delivery systems to fulfill the therapeutic potential of mRNA molecules (Cullis, P. R. and Jope, M. J., 2017, Mol Ther, 25:1467-1475; Samaridou, E., et al., 2020, Adv Drug Deliv Rev, 154-155:37-63). These LNP contain ionizable lipids (positively charged at pH<6.4), which aid in packaging the mRNA and endosomal escape. Such LNP were first approved in 2018 for siRNA, but became widely utilized in 2020, due to the LNP-mRNA platform for Moderna and Pfizer COVID-19 vaccines (Akinc, A., et al., 2010, Mol Ther, 18:1357-1364). The LNP-mRNA in these FDA-approved vaccines drive antigen expression, but do not actively target specific cells or organs.

The data presented herein demonstrates that LNP loaded with diverse mRNA cargos can access HSC in the BM niche in situ, with a single systemic injection. Delivery efficacy to long-term HSC in the BM niche is greatly increased by conjugation of a targeting moiety (anti-CD117 antibody). Here, data is presented showing that LNP loaded with a Cre mRNA cargo can induce durable genome edits in long-term HSC ex vivo and in vivo at, or above, the level required for cure of most NMHD. This approach can be translated to primary human cells, where it was able to achieve high rates of therapeutic base editing in hematopoietic cells from individuals with sickle cell disease. Additionally, it was demonstrated for the first time that a genetic medicine, targeted LNP-mRNA, can leverage the understanding of HSC biology (Mcll pathway dependence) to effect cellular state change in vivo with physiologic effects. This system was utilized to deplete HSC in vivo without genotoxic conditioning regimens that often result in pulmonary, liver, and reproductive toxicity (Campbell, C. J., et al., 2010, Blood, 116:1433-1442; Leiper, A. D., 2002, Br J Haematol, 118:23-43; Cohen, A., et al., 2008, Bone Marrow Transplant, 41(S2):S43-S48). Although this conditioning approach requires additional refinement to reduce toxicity, such as modifications to restrict LNP tropism and/or further limit gene expression in unintended cells, this has the capacity to replace current myeloablation approaches. These findings can potentially revolutionize gene therapy in two ways. First, the cure of monogenic disorders, including NMHD (hemoglobinopathies, congenital anemias or thrombocytopenias, and immunodeficiencies) and non-hematopoietic diseases (cystic fibrosis, metabolic disorders, myopathies) with a simple i.v. infusion of targeted genetic medicines. Second, effecting cell-type specific state changes in vivo with minimal risk will allow previously impossible manipulations of physiology. These novel delivery systems will translate the promise of decades of concerted genetic and biomedical research to cure a wide array of human diseases.

The materials and methods are now described.

### RNA Synthesis and Preparation of Targeted LNP-mRNA

Gene sequences for Firefly Luciferase (luc2), Cre Recombinase (cre), and enhanced Green Fluorescent Protein (eGFP) were sourced from SnapGene (www-snapgene-com/resources) software plasmids pGL4.10[luc2], pCMV6-Entry-Cre, and pEGFP, respectively, then codon-optimized. The ABE sequence (ABE8e-NRCH) and HBB^{S}-targeting sgRNA sequences were replicated from Newby et al. and the sgRNA was purchased from Synthego (Redwood City, CA, USA) (Newby, G. A., et al., 2021, Nature, 595:295-302). All sequences are in Table 1. Each gene coding sequence was cloned into an IVT-mRNA production template plasmid carrying a T7 promoter, 5′ and 3′ UTR elements, Kozak consensus sequence, and 101 poly(A) tail. DNA synthesis, cloning and industrial grade endotoxin-free plasmid preparation service was provided by GenScript (Piscataway, NJ, USA). IVT-mRNA was produced using linearized IVT template plasmid and the MEGAScript T7 kit (Thermo Fisher Scientific, AMB 13345, Waltham, MA, USA) and formulated with nucleoside-modified m1Ψ-5′-triphosphate (TriLink, N-1081, San Diego, CA) instead of UTP. 5′ Capping of the IVT-mRNAs were performed co-transcriptionally using the trinucleotide cap1 analog, CleanCap® Reagent AG (3′ OMe) (TriLink, N-7413, San Diego, CA, USA). Single-stranded IVT-mRNA was purified by cellulose purification, as previously described (Baiersdorfer, M., et al., 2019, Mol Ther Nucleic Acids, 15:26-35). All mRNAs were analyzed by agarose gel electrophoresis and were stored at −20° C. Cellulose purified m1Ψ-containing RNAs were encapsulated in LNP using a self-assembly process as previously described, briefly an ethanolic lipid mixture of ionizable cationic lipid, phosphatidylcholine, cholesterol, and polyethylene glycol-lipid was rapidly mixed with an aqueous solution containing the mRNA at acidic pH (Maier, M. A., et al., 2013, Mol Ther, 21:1570-1578). The RNA-loaded particles were characterized by dynamic light scattering using a Zetasizer Nano ZS (Malvern Instruments, Malvern, UK) and a Ribogreen assay. The mean hydrodynamic diameter of these LNP-mRNAs was approximately 80 nm with a polydispersity index of 0.02-0.06 and an encapsulation efficiency of ~95%. LNP used in this study are proprietary to Acuitas Therapeutics (Vancouver, BC, Canada). The ionizable cationic lipid and LNP composition are described in U.S. Pat. No. 10,221,127. To prepare antibody-targeted LNP-mRNA, LNP-mRNA were conjugated with purified rat anti-mouse CD117 (c-kit), clone 2B8 (BioLegend, 93235, San Diego, CA, USA) or mouse anti-human CD117 (c-kit), clone 104D2 (BioLegend, 95747, San Diego, CA, USA), mouse anti-mouse CD45.2, clone 104 (Biolegend, 92176, San Diego, CA, USA), and control isotype-matched IgG (Thermo Fisher Scientific, Rat IgG Isotype 31933, Mouse IgG Isotype 10400C, Waltham, MA, USA) via SATA-maleimide chemistry, as described previously (Parhiz, H., et al., 2018, J Control Release, 291:106-115). Briefly, LNP was modified with maleimide functioning groups (DSPE-PEG-mal) by a post-insertion technique. The antibody was functionalized with SATA (N-succinimidyl S-acetylthioacetate, 26102) from Thermo Fisher (Burlington, MA, USA) to introduce sulfhydryl groups allowing conjugation to maleimide. SATA was deprotected using 0.5 M hydroxylamine followed by removal of the unreacted components by Zeba spin desalting columns (Thermo Fisher Scientific, 89890, Waltham, MA, USA). The reactive sulfhydryl group on the antibody was then conjugated to maleimide moieties using thioether conjugation chemistry. Purification was carried out using Sepharose CL-4B gel filtration columns (MilliporeSigma, GE17-0150-01, Burlington, MA, USA). mRNA content was calculated by performing a modified Quant-iT RiboGreen RNA assay (Thermo Fisher Scientific, R11490, Waltham, MA, USA). After addition of the targeting ligand, all the targeted and non-targeted LNP preparations were kept at 4° C. and were used within three days of preparation.

### In Vitro Cell Transfection Studies and Luciferase Assay

LNP carrying reporter luciferase IVT-mRNA were added at increasing concentrations to the cells and incubated for 24 h. Plates were then washed with PBS, lysed in luciferase cell culture lysis reagent (Promega, E1531, Madison, WI, USA). The cell lysate was mixed with Firefly Luciferase Assay System substrate (Promega, E1500, Madison, WI, USA) and measured on a MiniLumat LB 9506 luminometer (Berthold/EG&G; Wallac, Bad Wildbad, Germany).

### Bioluminescence Imaging

C57BL/6J mice were i.v. injected with control IgG/LNP-Luc, CD117/LNP-Luc or CD117/LNP-Luc-miRts formulations. At 5 h post-injection, bioluminescence imaging was carried out as described previously using an IVIS Spectrum imaging system (Caliper Life Sciences, Waltham, MA, USA) (Kariko, K., et al., 2011, Nucleic Acids Res, 29:e142). D-luciferin sodium salt (Regis Technologies, 1-360243-200, Morton Grove, IL, USA) dissolved in PBS was administered to mice intraperitoneally at a dose of 150 mg/kg. After 5 min, the mice were euthanized; desired tissues were harvested, washed with PBS, and immediately placed on the imaging platform. Harvested femurs were slightly crushed by spatula to expose the bone marrow for imaging. Tissue luminescence was measured on the IVIS imaging system using an exposure time of 5 s or longer to ensure that the signal obtained was within operative detection range.

### Human Subjects

Specimens from patients with sickle cell disease were collected. Deidentified apheresis waste product from patients with SCD and CD34⁺ progenitor cells were isolated with the MACS MicroBead kit (Miltenyi).

### In Vitro Cell Treatment

Bone marrow cells were isolated from femurs of animals, after removal of muscle and connective tissues, by mechanical crushing, which maximizes cell recovery. BM cells were resuspended in 4% FBS (SH3007103) from Hyclone (Logan, UT) and PBS (10010023) from Gibco (Waltham, MA) and RBC were lysed using ACK lysis buffer (A1049201) from Gibco (Waltham, MA) at room temperature, according to manufacturer protocol, filtered through a 40 mm sterile strainer (431750) from Corning (Glendale, AZ) and washed with 4% FBS PBS solution. After lysis, cells were counted and assessed for viability by AOPI staining (CS2-0106), using a Cellometer Auto 2000 cell viability counter (Nexcelom Bioscience, Lawrence, MA) and seeded at a 1.5×10⁶/mL concentration in Stemspan SFEM (096000) from Stem Cell Technologies (Vancouver, BC, Canada) supplemented with 50 ng/mL mSCF (250-03), 6 ng/ml mIL-3 (213-13), and 10 ng/ml mIL-6 (216-16), all supplied by Peprotech (Cranbury, NJ), LNP formulations were added at the time of seeding and for up to 18 hours, depending upon assay.

### Human Erythroid Progenitor Cells (ErPC) Editing and Differentiation

Human CD34+ cells were isolated from blood products by immunomagnetic-separation using the CD34 MicroBead Kit (130-046-702) from Miltenyi Biotec Inc. (Auburn, CA). ErPC were obtained through expansion of CD34+ cells, using a culture system previously described and frozen after 6-10 days. Upon thawing cells were allowed to recover for 48 hrs and exposed to anti-human CD117/LNP-ABE and CD117/LNP-sgRNA formulation (3 to 10 μg/cells dose, at 1:1 weight ratio) at a 1.5×10⁶/mL cell concentration for 6 hours (Breda, L., et al., 2021, Mol Ther, 29:1625-1638). Cell viability was measured before and after treatment by AOPI staining. ErPC were let expand 24 prior to inducing differentiation. Differentiated erythroblasts were collected after 7 days, using a protocol previously described (Breda, L., et al., 2021, Mol Ther, 29:1625-1638).

### Sickling Assay

The degree of cell sickling was measured using a modified version of a method already described (Breda, L., et al., 2016, Blood, 128:113-1143). Briefly, 1×10⁶ differentiated erythroblasts were suspended in 100 μL of isotonic TES, supplemented with 10 mM glucose and 0.2% bovine serum albumin, in individual wells of a Costar polystyrene 96-well microplate (No 9017; Corning, Corning, NY). The microplate was then transferred to a Thermomixer R shaker-incubator (Eppendorf, Enfield, CT), and maintained under hypoxia (2.5% Oxygen gas, balance Nitrogen gas), with continuous agitation at 900 rpm, at 37° C. for 2 hours. At conclusion, aliquots (~20 μL) of each sample were collected in 2% glutaraldehyde solution for immediate fixation without exposure to air. Subsequently, fixed cell suspensions were introduced into specialized glass microslides (Dawn Scientific, Inc., Newark, NJ) for acquisition of bright field images (at 20× magnification) of single layer cells on a Zeiss Axiovert 200M inverted microscope (Carl Zeiss Microscopy, LLC, White Plains, NY), fitted with an Infinity 2 camera (Teledyne Luminera, Ottawa, ON, Canada) and the coupled Image Capture software (Asakura, T. and Mayberry, J., 1984, J Lab Clin Med, 104:987-994).

### Quantification of Hbb^{G-Makassar} Protein

Single chain quantification of individual globins was assessed by reverse-phase HPLC on clarified cell lysates obtained by disrupting cell pellets in water. Hemoglobin samples were injected in a Nexera apparatus from Shimadzu Scientific Instruments, Inc. (Columbia, MD) using a 250 mm×4.6 mm Aeris 3.6 μm C4 200A column from Phenomenex (Torrance, CA) and a gradient from 32% to 47% of 0.1% trifluoroacetic acid in acetonitrile in in 60 minutes, with UV detection at 215 nm. Serial dilutions of a solution with known concentrations of Hbb A-F—S—C(Helena Laboratories, Beaumont, TX) were used to generate a calibration curve, where the peak areas were plotted against the concentration values. Types and relative quantity of Hbs in samples were assessed by comparison to standard hemoglobin controls.

### Quantification of Base Editing

Genomic DNA was extracted with QIAamp DNA Mini Kit (56304) from Qiagen (Hilden, Germany) or QuickExtract™ DNA Extraction Solution (QE09050 and QE0905T), from Lucigen (Middelton, WI). Quantification of base editing was performed on 50 ng of genomic DNA upon amplification of the region that includes the binding site of the gRNA using KAPA2G Fast ReadyMix from Kapa Biosystem (Wilmington, MA, USA). Primers for amplification were HBB3-F 5′-TCTGGAGACGCAGGAAGAGA-3′ (SEQ ID NO:1) and HBB3-R 5′-CTGTCTCCACATGCCCAGTT-3′ (SEQ ID NO:2). After Sanger sequencing (Azenta), the editing percentage was calculated using EditR (for A>G conversion, Adenine Base editor targeting), following a workflow previously described (Kluesner, M. G., et al., 2018, CRISPR J 1:239-250).

### CFU Assay

CFU assay was conducted using reagents from Stem Cell Technologies. Bone marrow harvested from animals was seeded using a at 30,000 cells/well in complete Methocult media (M3434), in meniscus-free 6-well SmartDish plates (27371), using 16-gauge blunt end needles (28110) per directions from Stem Cell Technologies. Colonies were incubated for 2 weeks at 37° C. in CO₂ incubators. Colonies were imaged using Evos FL Auto (AMAFD 1000) manufactured by Life Technologies (Waltham, MA) microscope and analyzed using superimposed bright field and Texas Red filter images.

### Lung and Liver Perfusion

In preparation for organ perfusion mice were induced to general anesthesia by injected by IP injection of a 200 mg/kg ketamine (NDC #13985-584-10 From Vet One, Boise, ID) and 20 mg/kg xylazine (NDA #139-236, from Akorn Inc., Lake Forest, IL) solution in PBS. Upon reaching complete loss of footpad reflexes, the abdominal cavity was cut open and the ribcage was dissected to open the chest cavity. The heart was slowly infused with 10 mL of 1% FBS PBS solution using a 27 G×½ inch needle (305109) supplied by Beckton Dickinson (Franklin Lakes, NJ) upon interruption of portal vein flow. Lungs and liver were rapidly removed and first incubated in 1× Buffer S, provided with GentleMACS Lung Dissociation Kit (130-095-927), or Corning DMEM (10-017-CV) media, respectively. After initial homogenization, lung and liver were processed following manufacturing instructions for GentleMACS Lung or Liver (130-105-807) Dissociation Kits, purchased from Miltenyi Biotec (Bergisch Gladbach, North Rhine-Westphalia, Germany). Tissues were homogenized using GentleMACs Dissociator system (130-093-235), following the recommended programs. Cell pellets underwent 1-2 cycles of RBC lysis with ACK buffer, followed by ice cold PEB (phosphate-buffered saline (PBS) pH 7.2, 0.5% bovine serum albumin (12659) from Sigma Aldrich (St. Louis, MO), and 2 mM EDTA (Invitrogen) buffer washes. Cell number and viability were assessed by AOPI staining with Cellometer, as described.

### Flow Cytometry for Analyses for BM and Peripheral Blood

Monthly assessments of peripheral blood cell tdTomato marking were carried out by direct measurement of tdTomato expression in whole blood for the RBC compartment, or after RBC lysis, using ACK lysis buffer at room temperature, for the WBCs analyses. tdTomato expression in WBC was assessed using the following antibodies: violetFluor™ 450 CD3 (clone 17A2, 75-0032-U025) from Tonbo Biosciences (San Diego, CA), CD45R/B220-FITC (clone RA3-6B2, 103205) from BioLegend (San Diego, CA), Ly-6G/Gr1 PE-Cyanine7 (clone RB6-8C5, 565033) from BD Biosciences (Franklin Lakes, NJ), for detection of T, B cells and Granulocytes, respectively, while CD45.2 PerCP-Cyanine5.5 (clone 104, 45-0454-82) from eBioscience (Waltham, MA), CD45.1 APC (clone A20, 110713) BioLegend antibodies were used to discriminate proportion of donor versus recipient chimerism, respectively. Bone marrow samples obtained after crushing were treated with ACK lysis buffer to remove RBC prior to analyses. The following biotinylated antibodies were used to discriminate lineage committed cells: CD45R (13-0452-82)/CD8 (13-0081-82)/CD4 (3-0042-82)/CD127 (13-1271-82)/Gr1-Ly6G (13-5931-82)/Ter119 (13-5921-82) from eBiosciences. An APC-eFluor 780 streptavidin (47-4317-82) from Invitrogen was used to bind lineage committed cells pre-incubated with biotinylated lineage antibody cocktail. To discriminate LSK cells, an APC anti-CD117 (clone 2B8, 17-1171-82) from Invitrogen was utilized along with a Ly6A/E ((ScaI)-PE-Cyanine7 antibody (clone D7, 25-5981-82) from eBioscience). LT-HSC (LSK CD150+CD48−) were gated using a Pacific Blue anti-CD48 antibody (clone HM48-1, 103418) and a BV650 anti-CD150 antibody (clone TC15-12F12.2, 115931), both from BioLegend. Peripheral blood and spleen samples were acquired on a CytoFLEX S analyzer with 4 lasers (405, 488, 561, 638 nm) made by Beckman Coulter (Brea, CA), while bone marrow cells were acquired on a CytoFLEX LX analyzer with 6 lasers (375, 405, 488, 561, 638, 808 nm) also manufactured by Beckman Coulter. All acquisition data was analyzed using FlowJo software from Tree Star Inc (Ashland, OR).

### The Experimental Results are Now Described

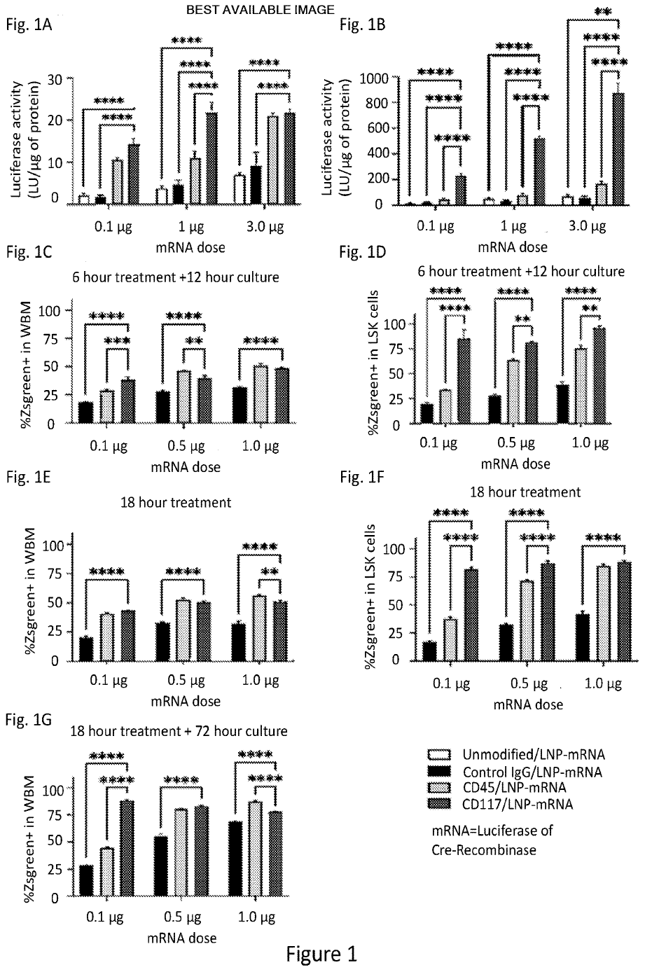

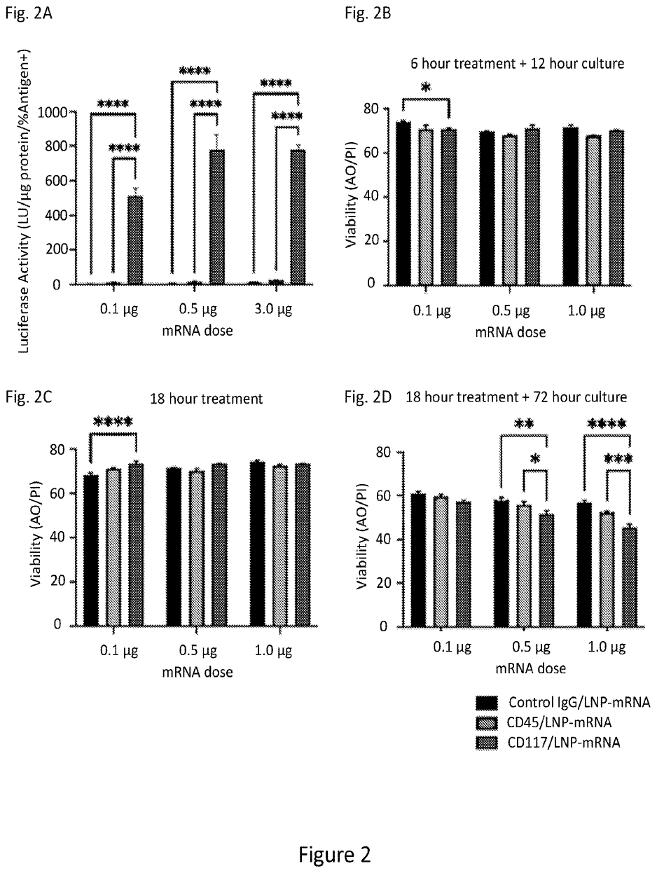

C57BL/6 whole BM (WBM) or lineage depleted BM cells (Lin⁻) were incubated in vitro with either unconjugated LNP encapsulating 0.1, 1, or 3 μg of nucleoside-modified luciferase mRNA (unmodified LNP-Luc), anti-CD45-conjugated LNP (CD45/LNP-Luc), anti-CD117-conjugated LNP (CD117/LNP-Luc), or isotype control IgG-conjugated LNP (control IgG/LNP-Luc). CD45/LNP and CD117/LNP were hypothesized to bind all hematopoietic-derived cells or stem and progenitor cells, respectively. Control IgG/LNP and unconjugated LNP were utilized as controls. The highest levels of luciferase activity in WBM were detected with CD117/LNP-Luc (FIG. 1A). Luciferase activity was further increased when Lin⁻ cells were treated with CD117/LNP-Luc (FIG. 1B). Increased activity of CD117/LNP-Luc in Lin⁻ cells was consistent with a 23-fold increase in the proportion of CD117⁺ in Lin-selected cells (2.8% CD117⁺ in WBM cells vs. 65% CD117⁺ in Lin⁻ cells). CD117/LNP luciferase activity was 500 and 700-fold higher than CD45/LNP luciferase activity in WBM and Lin, respectively, when normalized to the frequency of CD45 and CD117 positive cells in WBM and Lin⁻ (FIG. 2A). Normalized luciferase activity suggests that CD117 mediated targeting and delivery is superior to CD45 mediated targeting in vitro. This demonstrates efficient targeting and functional delivery of mRNA with CD117/LNP.

CD117/LNP encapsulating Cre recombinase mRNA (CD117/LNP-Cre) was used to test LNP-mediated genetic recombination in HSCs and persistence of the editing in conjunction with three reporter murine models. These murine models (Ai6, Ai9 and Ai14) are engineered with a Cre-responsive reporter allele comprised of a loxP-flanked STOP cassette preventing transcription of a CAG promoter-driven green or red fluorescent reporter gene (ZsGreen1 for Ai6 and tdTomato for Ai9 and Ai14, respectively) inserted into the Gt(ROSA)26Sor locus (Madisen, L., et al., 2010, Nat Neurosci, 13:133-140). The fraction of edited WBM cells (FIG. 1C), and the subset of edited Lin⁻Sca1⁺cKit⁺ cells (LSK) within the BM (FIG. 1D), exhibited a dose dependency (0.1 to 1 μg mRNA) when incubated with CD45/LNP-Cre and control IgG/LNP-Cre. The majority of LNP-mediated transfection occurred within 6 hours (FIG. 1C through FIG. 1F). Targeting rates in the LSK subset of WBM cells were consistently and significantly higher with CD117/LNP-Cre than with CD45/LNP-Cre or control IgG/LNP-Cre, suggesting saturation of cKit⁺ cells by CD117/LNP-Cre at the lowest dose tested (FIG. 1C and FIG. 1D). CD117/LNP-Cre showed higher efficacy in LSK cells at lower concentrations: treatment with 0.1 μg CD117/LNP-Cre was 2.5-fold more effective at targeting LSK cells compared to treatment with 0.1 μg CD45/LNP-Cre (FIG. 1D). There was no significant difference between targeted cell frequency in the LSK subset with the 0.1 μg and 0.5 μg dose or 0.5 μg and 1 μg dose. The media of cells treated for 18 hours with LNP-Cre and was also replaced and the cells kept for 3 additional days in culture to assess the maximum targeting achieved after exposing WBM to LNP. The rate of targeted cells increased over 3 days without additional LNP exposure (FIG. 1G): at a dose of 0.1 μg CD117/LNP-Cre, 88.5% WBM cells were ZsGreen+vs. 43.5% at 18 h (FIG. 1G and FIG. 1E), indicating additional mRNA translation, cre-mediated recombination, and ZsGreen⁺ transcription/translation occurred beyond the 18-hour LNP exposure. Importantly, LNP-Cre treatment had no consistent effect on cell viability across formulations, regardless of the targeting antibody (FIG. 2B through FIG. 2D). Hence, it was determined that the use of CD117/LNP-Cre was superior to that of CD45/LNP-Cre to modify HSCs and selected CD117-LNP-Cre for subsequent experiments.

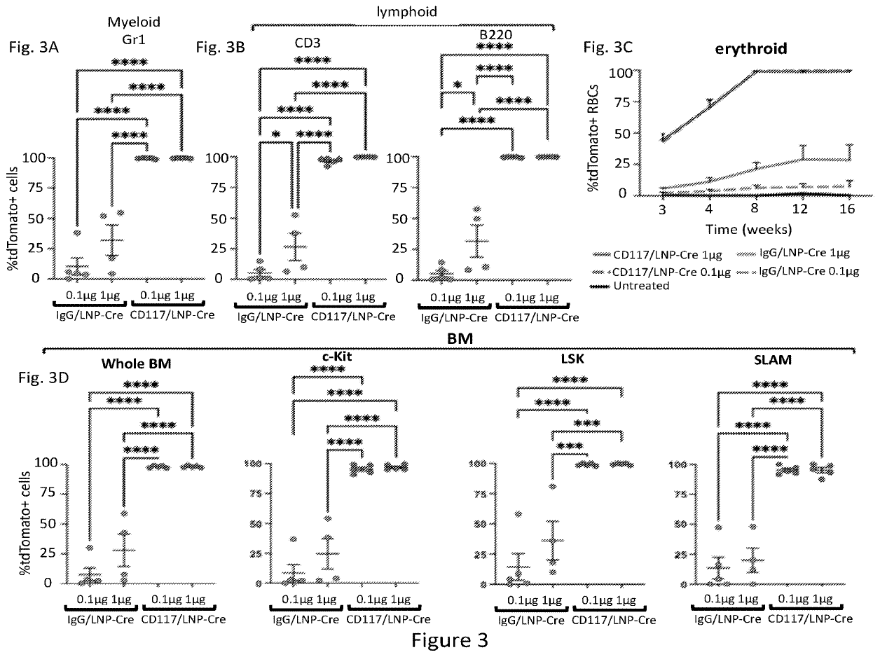

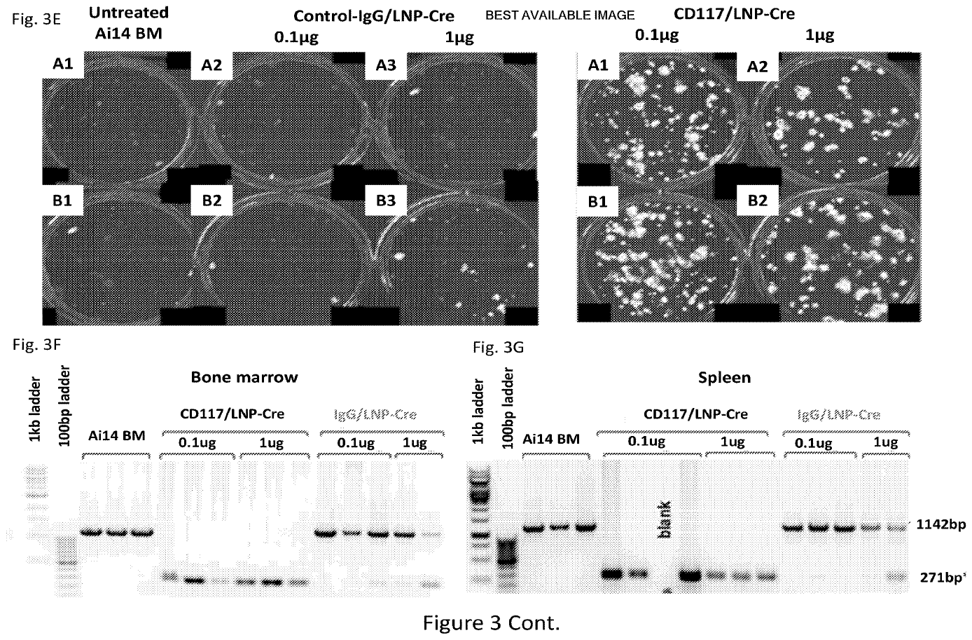

To evaluate multipotency in cells edited by CD117/LNP-Cre, lethally irradiated congenic C57BL/6 CD45.1 recipient mice were transplanted with Ai14 BM cells treated ex vivo with increasing doses of CD117/LNP-Cre and control IgG/LNP-Cre. Since HSCs give rise to all blood cell lineages, reporter gene expression was followed in peripheral blood cells over time and in the BM at the 4-month endpoint (FIG. 3). The percentage of CD117/LNP-Cre-mediated tdTomato positive Ai14 erythroid cells in recipient mice increased with time post-HSCT, consistent with engraftment of donor HSC (FIG. 3C). Mice had durable editing in all lineages, specifically myeloid cells (Gr1+, FIG. 3A), lymphoid cells (CD3+ and B220+, FIG. 3B), and erythroid cells (FIG. 3C) at 4 months post-HSCT, consistent with genome editing of multipotent HSC.

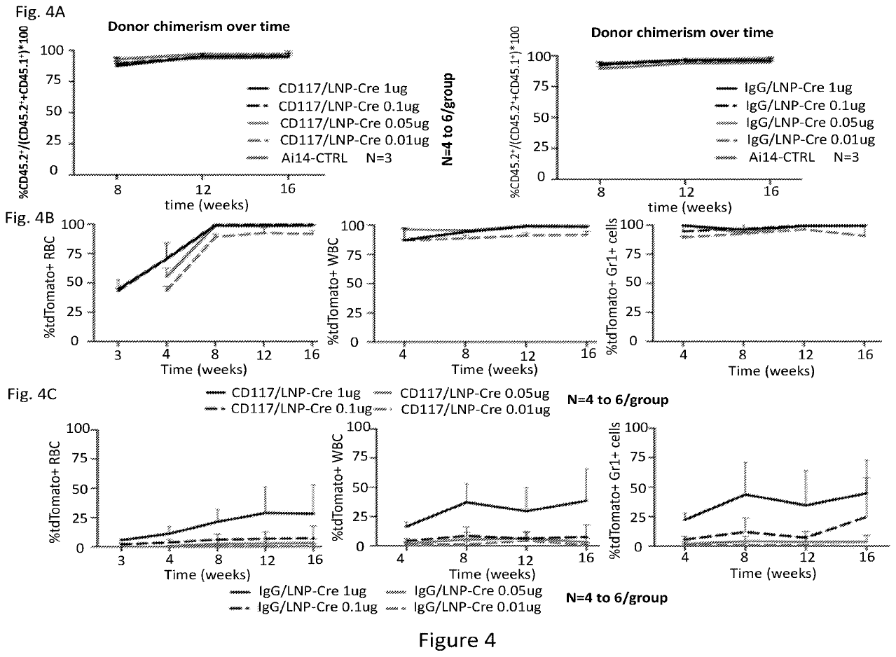

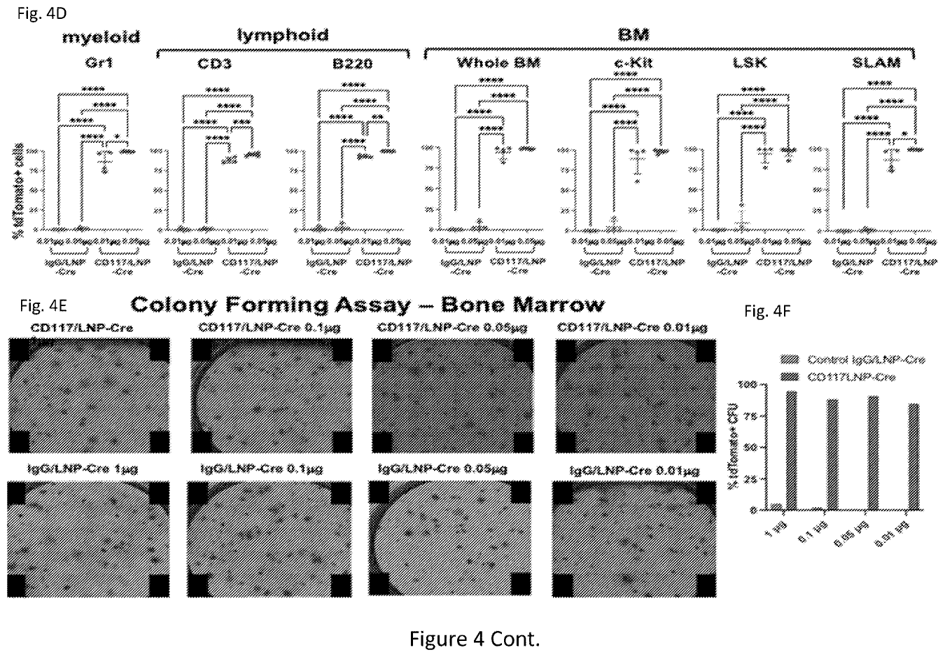

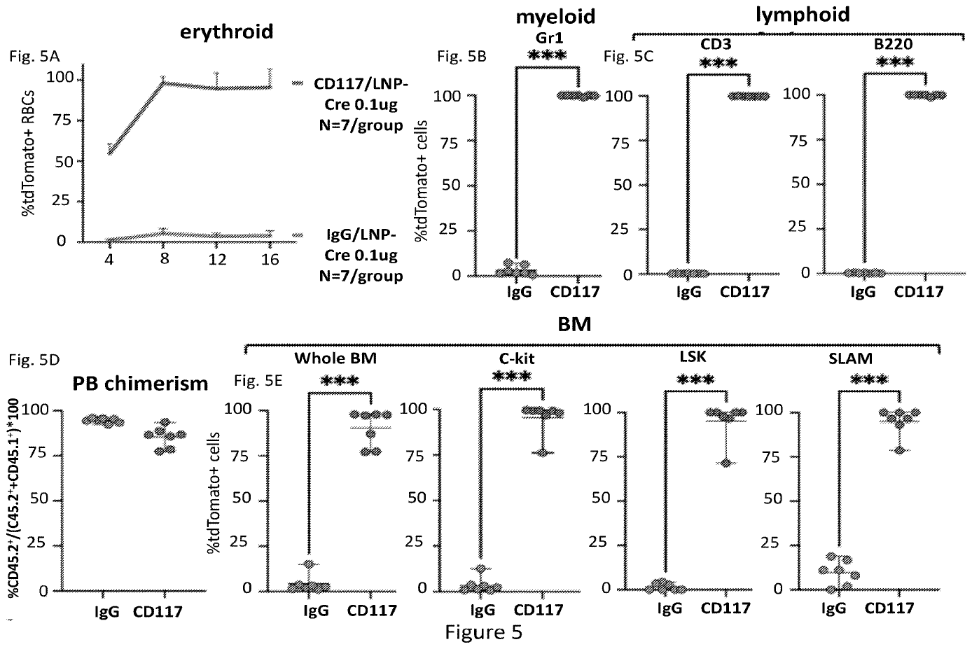

Editing rates in long-term HSC (LT-HSC, LSK CD150+CD48− aka SLAM) was 95% at the 0.1 and 1 μg mRNA dose with CD117/LNP-Cre compared to 13.5 and 20% with control IgG/LNP-Cre, respectively (FIG. 3D), which was similar to that seen in the whole BM (WBM), the c-Kit⁺, and the LSK subsets, respectively. Donor chimerism was consistently high among all groups (over 94% at 4 months, FIG. 4A). Gene editing rates of ex vivo treated bone marrow cells were dose dependent (FIG. 4B and FIG. 4C). RBC and leukocyte editing rates with CD117/LNP-Cre were ≥99% at 0.05, 0.1, and 1 μg mRNA dose, and 91.8% at the 0.01 μg dose (FIG. 3A through FIG. 3C and FIG. 4D). By comparison, targeting mediated by control IgG/LNP-Cre was near 0% at 0.01 μg (FIG. 4C and FIG. 4D). tdTomato⁺Gr1⁺ cells had the fastest rise (FIG. 4B and FIG. 4C), which is expected given their rapid turnover of 2-3 days. BM cells harvested from these animals showed similar editing rates in colony forming assays, a functional assay for clonogenic potential, thus corroborating the flow cytometry results of LT-HSC (FIG. 3E, FIG. 4E, and FIG. 4F). At four months post-HSCT, splenocytes had genome editing levels comparable to those in the WBM (FIG. 3F and FIG. 3G), consistent with migration of edited BM-derived cells to the spleen. To assess stem cell potential of ex vivo edited bone marrow cells, secondary transplants were performed using the BM from two primary chimeras that were recipients of Ai14 BM cells treated ex vivo with either CD117/LNP-Cre or control IgG/LNP-Cre (0.1 μg dose of mRNA). Editing levels in secondary chimeras phenocopied those observed in the primary transplantation, including sustained editing in the LT-HSC subset and editing in multiple hematopoietic lineages (FIG. 5).

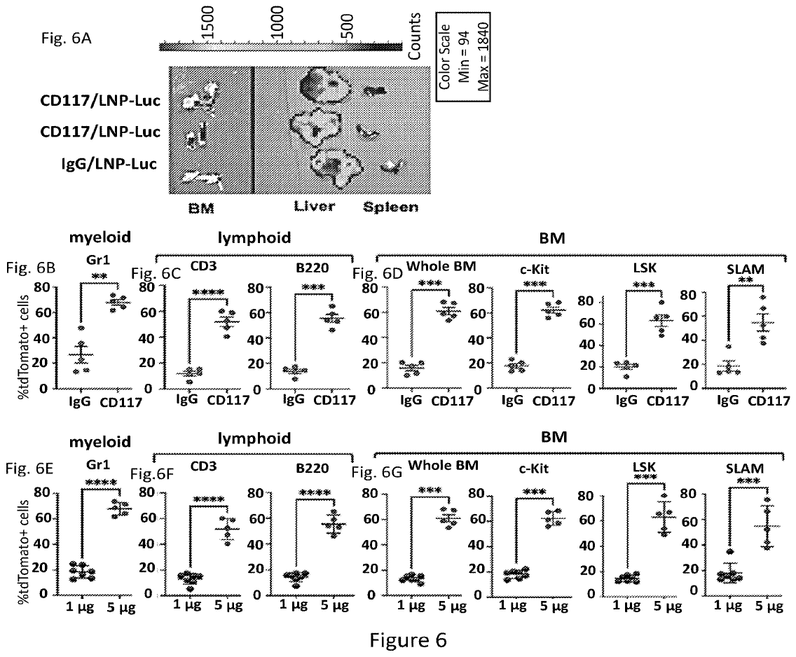

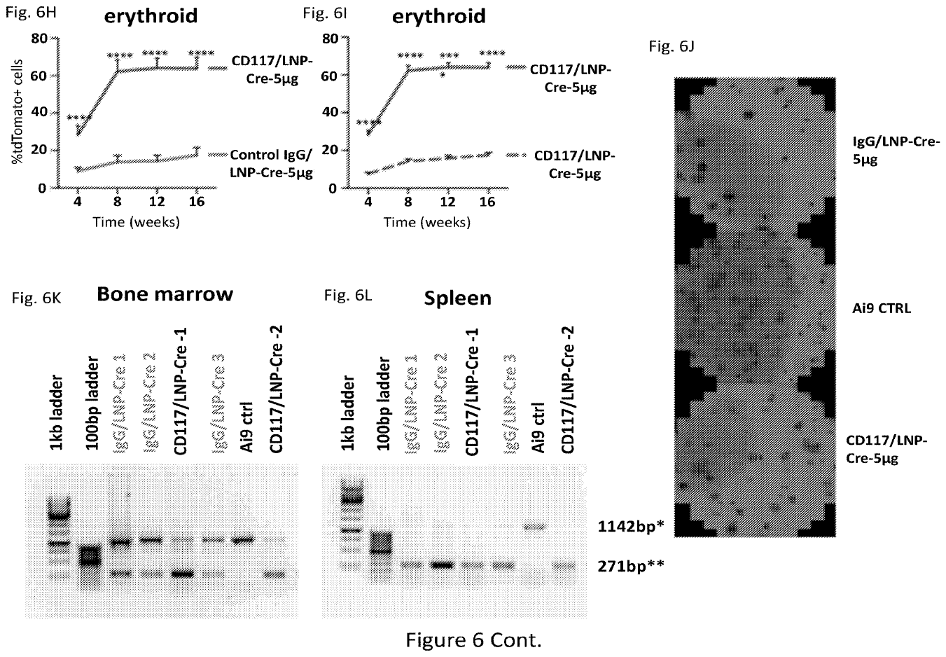

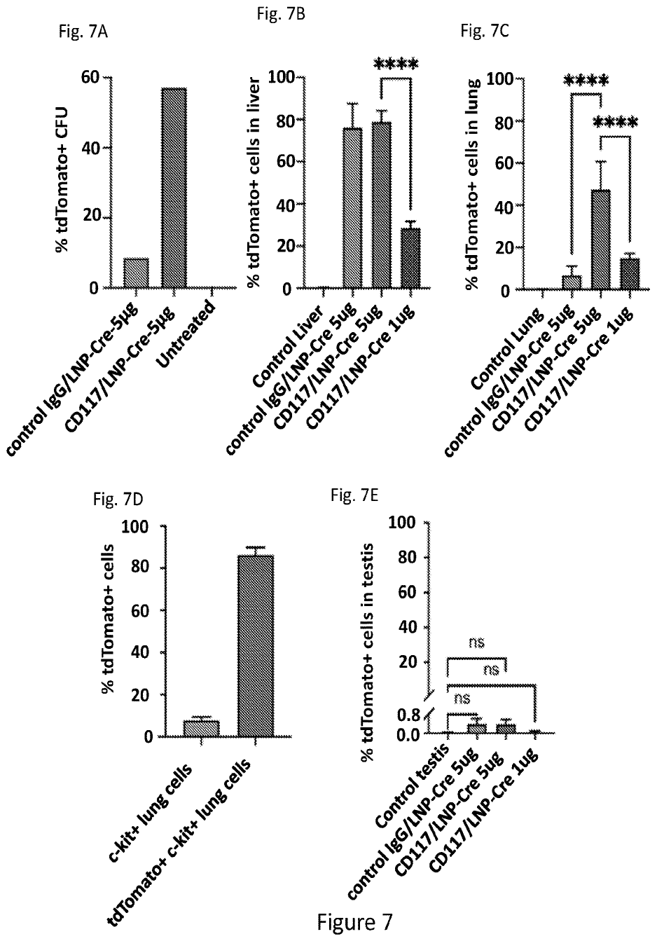

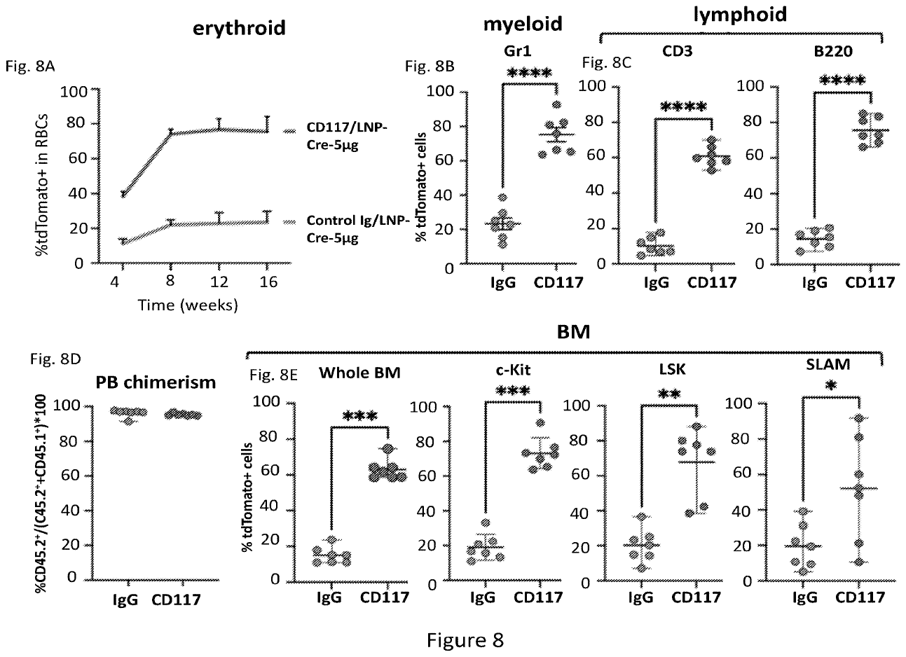

Given the near complete targeting of LT-HSC ex vivo with CD117/LNP-Cre and our prior ability to target lung endothelial and T cells in vivo, it was hypothesized that LT-HSC could be targeted in vivo, as well (Parhiz, H., et al., 2018, J Control Release, 291:106-115; Marcos-Contreras, O. A., et al., 2020, Proc Natl Acad Sci USA 117:3405-3414; Tombacz, I., et al., 2021, Mol Ther, 29:3293-33041 Rurik, J. G., et al., 2022, Science, 375:91-96). Intravenous (i.v.) administration of CD117/LNP-Luc generated luciferase activity in the femur at 24 hours, whereas IgG/LNP-Luc did not (FIG. 6A). Of note, both control IgG/LNP-Luc and CD117/LNP-Luc showed comparable luciferase activity in the liver, as LNP bind ApoE and are non-specifically targeted to the LDL receptor, which is expressed on hepatocytes (Akinc, A., et al., 2010, Mol Ther, 18:1357-1364). In vivo multilineage editing was tested by quantifying tdTomato expression in peripheral blood cells of i.v. CD117/LNP-Cre treated animals over time (up to 4 months) and tdTomato expression in bone marrow at 4 months. Peripheral blood multilineage and BM editing was evaluated at 16 weeks and, specifically, in LT-HSC. At the same dose (5 μg) CD117/LNP-Cre treated mice had consistently higher editing in all peripheral blood lineages (FIG. 6B and FIG. 6C), and importantly 3-fold higher editing in LT-HSC (55 vs 19%, respectively) compared to that observed in control IgG/LNP-Cre treated mice (FIG. 6D). HSC editing after in vivo treatment with CD117/LNP-Cre was dose dependent in peripheral blood and bone marrow at 16 weeks with a 5.5-fold increase in the percentage of gene-edited LT-HSC with 5 μg versus 1 μg (FIG. 6E through FIG. 6G). LNP-Cre in vivo editing led to appearance of edited RBC and WBC (not shown) with similar kinetics to transplantation of ex vivo treated bone marrow (FIG. 6H and FIG. 6I). At 4-months post-treatment with CD117/LNP-Cre, marking of HSC was confirmed by visual inspection of tdTomato⁺CFU (FIG. 6J and FIG. 7A), while Cre-mediated genomic deletion in the BM and splenic DNAs was confirmed by PCR (FIG. 6K and FIG. 6L). To demonstrate that these modifications targeted functional HSC, chimeras were generated by transplanting irradiated congenic (C₅₇BL/6 CD45.1) recipients with BM from two mice four months post-in vivo treatment with a 5 μg dose of CD117 or control IgG/LNP-Cre. Assessment of the hematopoietic-derived lineages, including LT-HSC in the BM, in these chimeras recapitulated editing found in the donor cells (FIG. 8).

To quantify non-specific cellular uptake, tdTomato expression levels in lung and liver cells were compared 4-months after in vivo treatment with a single dose of CD117/LNP-Cre (1 and 5 μg dose) or control IgG/LNP-Cre (5 μg dose). At 5 μg, liver editing was high (76-79% of cells), and editing was comparable between the two treatments (FIG. 7B), consistent with known non-specific ApoE and LDL receptor axis mediated LNP mediated uptake (Akinc, A., et al., 2010, Mol Ther, 18:1357-1364). In the lung, tdTomato expression mediated by CD117/LNP-Cre delivery was significantly higher (7-fold) than that of mice injected with control IgG/LNP-Cre (FIG. 7C). Editing observed in the perfused lung was 3-fold higher with 5 μg of CD117/LNP-Cre compared to 1 μg. This effect was partly “on-target” editing: ~8% of lung cells were ckit⁺ and ~90% of lung cKit⁺ cells were edited (FIG. 7D). Cells collected from the testis were also analyzed and did not show significant variations from baseline levels in control mice (FIG. 7E). Additionally, none of 89 offspring sired by male mice treated with CD117 or control IgG/LNP-Cre in vivo expressed tdTomato, N=38 pups from control-IgG male group and N=50 pups from CD117 male group.

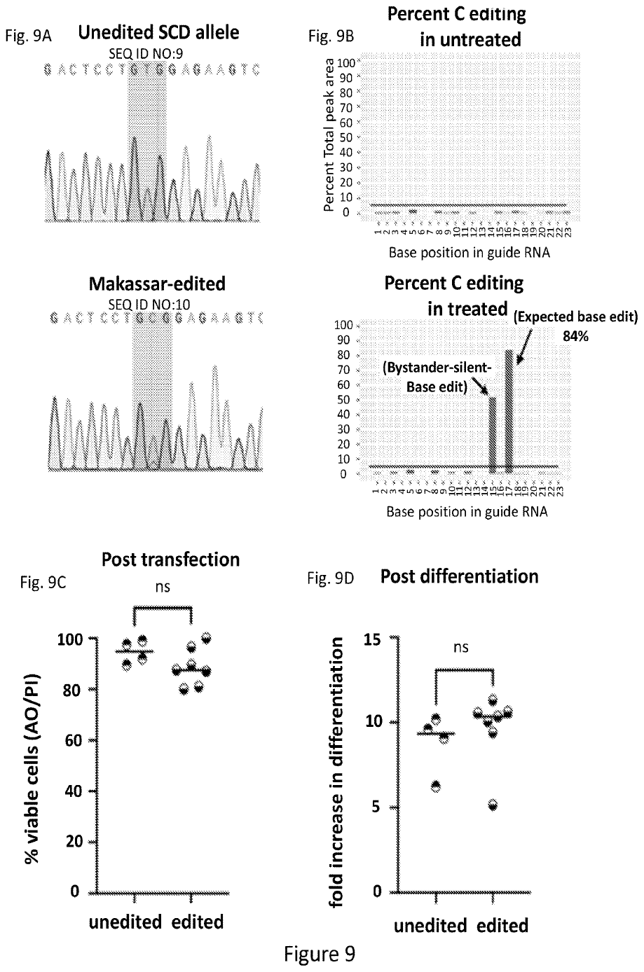

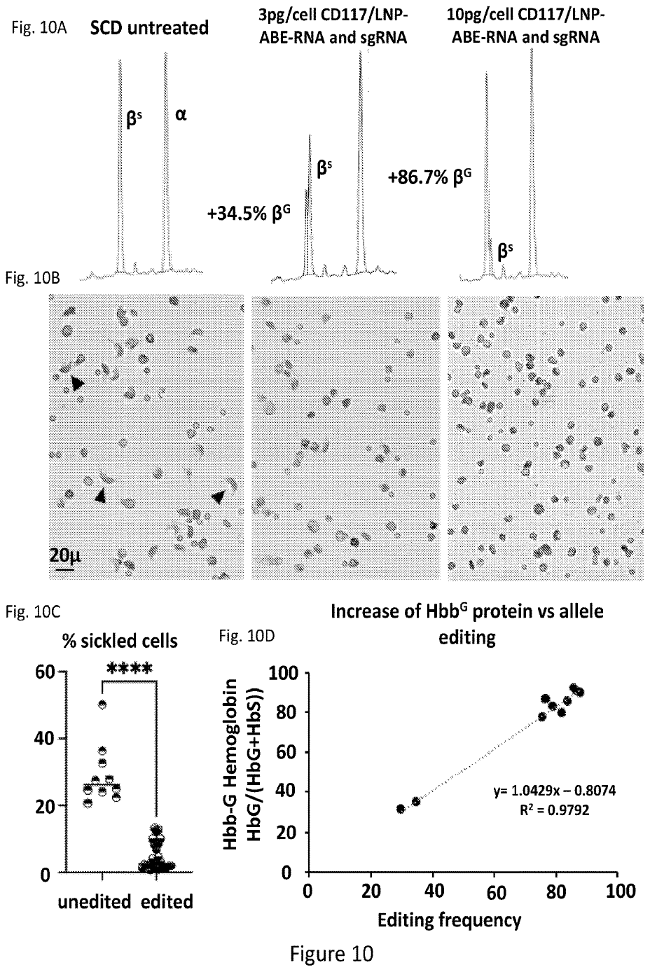

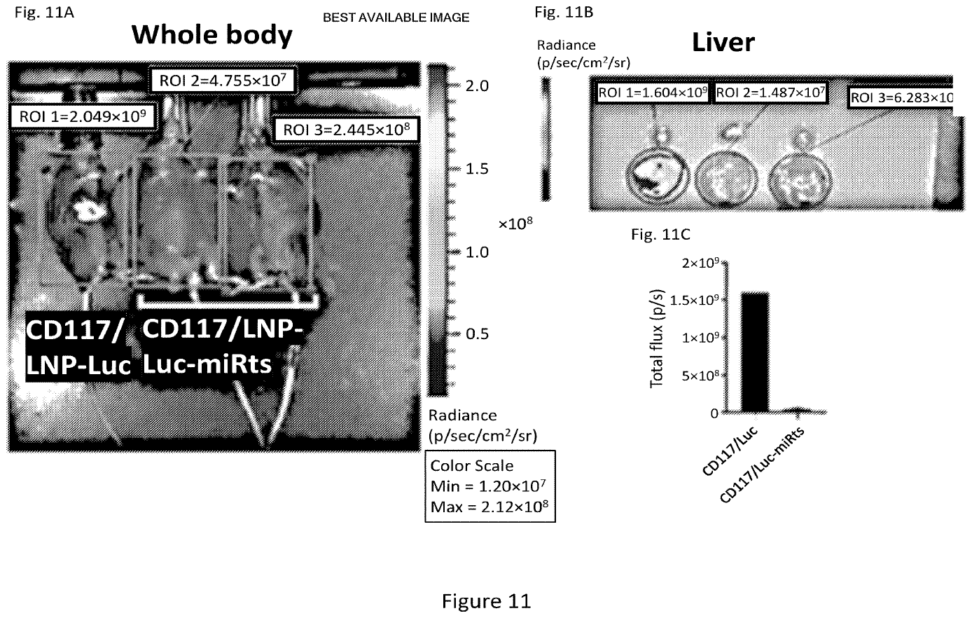

To assess the feasibility of using this platform for therapeutic human genome editing, targeting was adapted to human CD117 and utilized LNP containing mRNA encoding a cas9 adenine base editor (ABE) and LNP carrying a single guide RNA (sgRNA) targeted to the beta-globin sickle cell mutation. Adenine base editing of the A to G leads to conversion of the pathogenic E6V (HBB^{S}) mutation to a non-pathogenic E6A variant (HBB^{G-Makassar}) (Newby, G. A., et al., 2021, Nature, 595:295-302). This therapeutic strategy was applied to convert pathogenic sickle hemoglobin (HBB^{S}) to non-pathogenic G-Makassar hemoglobin (HBB^{G}) on 4 sickle cell specimens from separate donors (FIG. 9A and FIG. 9B). It was found that an excess of sgRNA to ABE mRNA containing LNPs lead to efficient editing with highest rates (88%) at 10 μg/cell dose (FIG. 10A). This led to a corresponding increase in HBB^{G} (up to 91.7%) and HBB^{S} decrease after in vitro erythroid differentiation, and a nearly complete absence of sickled cells upon exposure of the erythroblasts to hypoxic conditions (FIG. 10B and FIG. 10C). Editing levels and increase of HBB^{G} were directly correlated (FIG. 10D). It was observed that LNP doses from 3 μg/cell up to 10 μg/cell did not alter viability and proliferation rate of erythroid progenitor cells in vitro (FIG. 9C and FIG. 9D).

| | |
|---|---|
| eGFP | ATGGTGTCTAAGGGCGAGGAATTGTTTACAGGTGTGGTGCCC |
| | ATCCTGGTGGAGCTTGATGGCGATGTAAATGGACACAAATTC |
| | TCCGTTAGTGGGGAAGGCGAAGGGGATGCCACCTACGGTAA |
| | GCTTACGCTGAAATTCATCTGCACCACTGGTAAACTCCCCGT |
| | GCCATGGCCAACCCTGGTCACGACCCTTACTTATGGGGTGCA |
| | GTGTTTTTCAAGGTACCCCGACCATATGAAACAACATGATTT |
| | CTTCAAGTCCGCCATGCCGGAGGGGTACGTCCAGGAGAGAA |
| | CAATCTTTTTCAAAGATGACGGGAACTACAAGACTCGCGCA |
| | GAAGTCAAGTTTGAGGGAGACACTCTCGTAAACCGAATTGA |
| | ACTGAAAGGAATTGACTTTAAGGAAGACGGTAATATACTGG |
| | GCCACAAGCTGGAGTATAATTATAACAGCCATAATGTGTATA |
| | TCATGGCAGACAAGCAAAAGAACGGCATTAAAGTGAACTTC |
| | AAGATCCGTCACAATATCGAGGATGGCAGCGTCCAGCTGGC |
| | TGACCACTACCAGCAGAACACACCTATTGGAGACGGCCCAG |
| | TTTTACTACCTGACAACCACTATCTCAGTACACAGAGCGCCC |
| | TCTCTAAGGACCCTAATGAAAAGCGGGATCATATGGTTTTAC |
| | TGGAGTTTGTCACAGCTGCGGGAATAACCTTGGGCATGGATG |
| | AGTTGTACAAATGA (SEQ ID NO: 3) |
| Luc2 | ATGGAAGACGCCAAGAACATTAAGAAGGGCCCTGCACCCTT |
| | CTACCCACTGGAAGACGGTACTGCAGGGGAGCAGCTGCACA |
| | AGGCCATGAAGCGGTATGCCCTCGTTCCTGGCACTATCGCCT |
| | TCACAGATGCCCACATCGAAGTAGATATCACCTATGCTGAGT |
| | ACTTTGAGATGAGTGTGAGACTGGCAGAGGCAATGAAACGT |
| | TATGGACTGAACACCAACCATAGAATCGTAGTGTGCTCTGAG |
| | AACAGCTTGCAGTTCTTCATGCCTGTCTTAGGAGCACTGTTC |
| | ATAGGCGTCGCCGTGGCACCAGCCAATGACATTTACAATGA |
| | GAGGGAGCTCCTGAATAGTATGGGCATAAGCCAGCCAACAG |
| | TGGTTTTCGTTTCCAAGAAAGGGCTTCAAAAAATCCTGAATG |
| | TGCAAAAGAAGCTCCCTATCATCCAGAAGATCATTATAATGG |
| | ACTCAAAGACTGATTACCAGGGCTTCCAGTCCATGTATACAT |
| | TTGTCACCAGCCACCTTCCACCAGGCTTCAATGAATATGATT |
| | TCGTGCCTGAGTCATTTGACAGGGACAAAACAATTGCACTCA |
| | TTATGAATTCTTCCGGGTCAACAGGTTTGCCCAAGGGGGTGG |
| | CGCTACCACACAGAACGGCGTGTGTGCGCTTTAGCCATGCTC |
| | GAGACCCCATCTTCGGGAATCAGATCATTCCCGACACTGCCA |
| | TCTTGTCTGTCGTCCCTTTTCACCATGGTTTCGGTATGTTCAC |
| | CACGTTGGGCTACCTGATCTGTGGTTTCCGGGTAGTACTGAT |
| | GTACAGGTTTGAAGAAGAGCTCTTCCTGCGGAGCCTACAGG |
| | ACTACAAGATCCAGAGCGCACTGCTGGTGCCCACCCTTTTTT |
| | CGTTCTTTGCCAAATCCACCCTGATTGATAAATATGACCTAT |
| | CCAACCTTCATGAGATAGCATCTGGAGGTGCTCCTCTGAGTA |
| | AAGAAGTCGGAGAAGCTGTAGCCAAGAGGTTCCACCTGCCA |
| | GGCATTCGCCAAGGATATGGCCTGACAGAGACTACAAGTGC |
| | CATTTTAATAACTCCAGAGGGAGATGACAAGCCTGGGGCTG |
| | TGGGCAAAGTTGTTCCGTTCTTCGAAGCTAAGGTGGTTGACC |
| | TGGACACAGGAAAAACCCTGGGCGTCAACCAGCGTGGGGAA |
| | CTCTGCGTCCGAGGGCCCATGATCATGTCTGGCTACGTGAAC |
| | AACCCCGAGGCCACCAATGCCCTCATTGACAAGGATGGCTG |
| | GCTCCATTCAGGAGACATTGCCTACTGGGACGAGGATGAAC |
| | ACTTTTTTATTGTGGACAGGCTCAAGTCGCTTATCAAGTACA |
| | AAGGCTACCAGGTGGCTCCTGCTGAATTGGAATCCATCTTAC |
| | TTCAGCACCCCAACATATTTGATGCGGGTGTGGCCGGTCTAC |
| | CGGATGATGATGCAGGAGAGCTGCCCGCTGCTGTTGTTGTGC |
| | TGGAGCATGGTAAGACCATGACTGAGAAGGAGATTGTGGAC |
| | TATGTAGCGTCTCAAGTCACGACCGCTAAAAAACTAAGAGG |
| | GGGTGTGGTCTTTGTGGATGAGGTCCCAAAAGGATTGACTGG |
| | GAAGCTGGATGCTCGCAAAATAAGAGAAATCCTCATCAAAG |
| | CAAAGAAGGGAGGGAAAATTGCTGTCTGA (SEQ ID NO: 4) |
| Cre | ATGTCTAATCTCCTCACTGTGCATCAGAATCTTCCAGCTTTAC |
| | CGGTAGACGCCACGTCTGATGAAGTGCGCAAAAATCTCATG |
| | GACATGTTCAGGGACCGGCAAGCCTTCAGTGAGCACACATG |
| | GAAGATGTTGTTGTCTGTGTGTCGCTCCTGGGCTGCCTGGTG |
| | CAAACTTAACAACAGGAAGTGGTTCCCTGCAGAGCCTGAGG |
| | ACGTCAGAGACTATCTGCTCTACTTGCAAGCACGAGGACTCG |
| | CGGTAAAGACCATCCAGCAGCACCTGGGCCAGCTGAACATG |
| | CTGCACAGGAGGTCTGGGCTGCCCCGACCAAGTGACTCAAA |
| | TGCTGTGTCTCTGGTCATGAGACGCATCCGCAAGGAGAATGT |
| | GGATGCCGGAGAACGAGCCAAGCAGGCTCTGGCTTTTGAAC |
| | GGACAGACTTTGATCAGGTGAGATCCCTGATGGAGAACTCA |
| | GATAGATGCCAGGACATCCGGAACCTGGCCTTTCTTGGGATA |
| | GCCTACAACACCTTGCTGAGGATAGCAGAAATTGCCAGAAT |
| | TCGGGTCAAGGACATTAGCAGGACAGATGGGGGCAGGATGC |
| | TCATCCACATTGGCCGGACTAAAACCCTTGTTTCAACTGCAG |
| | GCGTGGAAAAAGCCTTGAGCTTAGGTGTCACCAAGCTGGTG |
| | GAGAGATGGATCAGCGTCTCCGGAGTTGCAGACGACCCAAA |
| | TAATTATCTCTTCTGTCGTGTTCGGAAGAACGGAGTTGCAGC |
| | GCCCTCGGCTACCAGCCAACTAAGCACGAGAGCTCTGGAGG |
| | GCATTTTTGAGGCCACTCATCGCCTGATCTATGGAGCAAAAG |
| | ATGACTCCGGGCAGAGATACCTGGCATGGAGTGGTCATAGT |
| | GCTCGTGTCGGTGCTGCAAGAGATATGGCCCGGGCTGGGGTT |
| | TCCATACCTGAAATCATGCAGGCTGGTGGCTGGACAAACGT |
| | GAACATTGTGATGAACTACATCAGGAATCTAGATTCTGAGAC |
| | AGGAGCCATGGTGCGATTACTGGAAGATGGCGATTGA |
| | (SEQ ID NO: 5) |
| miRts | TGATAATAGCAAACACCATTGTCACACTCCAGCTGGAGCCTC |
| | GGTGGCCATGCTTCTTGCCCCTTGGGCCCAAACACCATTGTC |
| | ACACTCCATCCCCCCAGCCCCTCCTCCCCTTCCTGCACCCGT |
| | ACCCCCCAAACACCATTGTCACACTCCAGTGGTCTTTGAATA |
| | AAGTCTGAGTGGGCGGC (SEQ ID NO: 6) |
| ABE8e- | ATGAAACGGACAGCCGACGGAAGCGAGTTCGAGTCACCAAA |
| NRCH | GAAGAAGCGGAAAGTCTCTGAGGTGGAGTTTTCCCACGAGT |
| | ACTGGATGAGACATGCCCTGACCCTGGCCAAGAGGGCACGG |
| | GATGAGAGGGAGGTGCCTGTGGGAGCCGTGCTGGTGCTGAA |
| | CAATAGAGTGATCGGCGAGGGCTGGAACAGAGCCATCGGCC |
| | TGCACGACCCAACAGCCCATGCCGAAATTATGGCCCTGAGA |
| | CAGGGCGGCCTGGTCATGCAGAACTACAGACTGATTGACGC |
| | CACCCTGTACGTGACATTCGAGCCTTGCGTGATGTGCGCCGG |
| | CGCCATGATCCACTCTAGGATCGGCCGCGTGGTGTTTGGCGT |
| | GAGGAACTCAAAAAGAGGCGCCGCAGGCTCCCTGATGAACG |
| | TGCTGAACTACCCCGGCATGAATCACCGCGTCGAAATTACCG |
| | AGGGAATCCTGGCAGATGAATGTGCCGCCCTGCTGTGCGATT |
| | TCTATCGGATGCCTAGACAGGTGTTCAATGCTCAGAAGAAG |
| | GCCCAGAGCTCCATCAACTCCGGAGGATCTAGCGGAGGCTC |
| | CTCTGGCTCTGAGACACCTGGCACAAGCGAGAGCGCAACAC |
| | CTGAAAGCAGCGGGGGCAGCAGCGGGGGGTCAGACAAGAA |
| | GTACAGCATCGGCCTGACCATCGGCACCAACTCTGTGGGCTG |
| | GGCCGTGATCACCGACGAGTACAAGGTGCCCAGCAAGAAAT |
| | TCAAGGTGCTGGGCAACACCGACCGGCACAGCATCAAGAAG |
| | AACCTGATCGGAGCCCTGCTGTTCGACAGCGGCGAAACAGC |
| | CGAGGCCACCCGGCTGAAGAGAACCGCCAGAAGAAGATACA |
| | CCAGACGGAAGAACCGGATCTGCTATCTGCAAGAGATCTTC |
| | AGCAACGAGATGGCCAAGGTGGACGACAGCTTCTTCCACAG |
| | ACTGGAAGAGTCCTTCCTGGTGGAAGAGGATAAGAAGCACG |
| | AGCGGCACCCCATCTTCGGCAACATCGTGGACGAGGTGGCC |
| | TACCACGAGAAGTACCCCACCATCTACCACCTGAGAAAGAA |
| | ACTGGTGGACAGCACCGACAAGGCCGACCTGCGGCTGATCT |
| | ATCTGGCCCTGGCCCACATGATCAAGTTCCGGGGCCACTTCC |
| | TGATCGAGGGCGACCTGAACCCCGACAACAGCGACGTGGAC |
| | AAGCTGTTCATCCAGCTGGTGCAGACCTACAACCAGCTGTTC |
| | GAGGAAAACCCCATCAACGCCAGCGGCGTGGACGCCAAGGC |
| | CATCCTGTCTGCCAGACTGAGCAAGAGCAGACGGCTGGAAA |
| | ATCTGATCGCCCAGCTGCCCGGCGAGAAGAAGAATGGCCTG |
| | TTCGGAAACCTGATTGCCCTGAGCCTGGGCCTGACCCCCAAC |
| | TTCAAGAGCAACTTCGACCTGGCCGAGGATGCCAAACTGCA |
| | GCTGAGCAAGGACACCTACGACGACGACCTGGACAACCTGC |
| | TGGCCCAGATCGGCGACCAGTACGCCGACCTGTTTCTGGCCG |
| | CCAAGAACCTGTCCGACGCCATCCTGCTGAGCGACATCCTGA |
| | GAGTGAACACCGAGATCACCAAGGCCCCCCTGAGCGCCTCT |
| | ATGGTGAAGAGATACGACGAGCACCACCAGGACCTGACCCT |
| | GCTGAAAGCTCTCGTGCGGCAGCAGCTGCCTGAGAAGTACA |
| | AAGAGATTTTCTTCGACCAGAGCAAGAACGGCTACGCCGGC |
| | TACATTGACGGCGGAGCCAGCCAGGAAGAGTTCTACAAGTT |
| | CATCAAGCCCATCCTGGAAAAGATGGACGGCACCGAGGAAC |
| | TGCTCGTGAAGCTGAACAGAGAGGACCTGCTGCGGAAGCAG |
| | CGGACCTTCGACAACGGCATTATCCCCCACCAGATCCACCTG |
| | GGAGAGCTGCACGCCATTCTGCGGCGGCAGGGCGATTTTTAC |
| | CCATTCCTGAAGGACAACCGGGAAAAGATCGAGAAGATCCT |
| | GACCTTCCGCATCCCCTACTACGTGGGCCCTCTGGCCAGGGG |
| | AAACAGCAGATTCGCCTGGATGACCAGAAAGAGCGAGGAAA |
| | CCATCACCCCCTGGAACTTCGAGGAAGTGGTGGACAAGGGC |
| | GCTTCCGCCCAGAGCTTCATCGAGCGGATGACCAACTTCGAT |
| | AAGAACCTGCCCAACGAGAAGGTGCTGCCCAAGCACAGCCT |
| | GCTGTACGAGTACTTCACCGTGTATAACGAGCTGACCAAAGT |
| | GAAATACGTGACCGAGGGAATGAGAAAGCCCGCCTTCCTGA |
| | GCGGCGAGCAGAAAAAGGCCATCGTGGACCTGCTGTTCAAG |
| | ACCAACCGGAAAGTGACCGTGAAGCAGCTGAAAGAGGACTA |
| | CTTCAAGAAAATCGAGTGCTTCGACTCCGTGGAAATCTCCGG |
| | CGTGGAAGATCGGTTCAACGCCTCCCTGGGCACATACCACG |
| | ATCTGCTGAAAATTATCAAGGACAAGGACTTCCTGGACAAT |
| | GAGGAAAACGAGGACATTCTGGAAGATATCGTGCTGACCCT |
| | GACACTGTTTGAGGACAGAGAGATGATCGAGGAACGGCTGA |
| | AAACCTATGCCCACCTGTTCGACGACAAAGTGATGAAGCAG |
| | CTGAAGCGGCTGAGATACACCGGCTGGGGCAGGCTGAGCCG |
| | GAAGCTGATCAACGGCATCCGGGACAAGCAGTCCGGCAAGA |
| | CAATCCTGGATTTCCTGAAGTCCGACGGCTTCGCCAACAGAA |
| | ACTTCATGCAGCTGATCCACGACGACAGCCTGACCTTTAAAG |
| | AGGACATCCAGAAAGCCCAGGTGTCCGGCCAGGGCGATAGC |
| | CTGCACGAGCACATTGCCAATCTGGCCGGCAGCCCCGCCATT |
| | AAGAAGGGCATCCTGCAGACAGTGAAGGTGGTGGACGAGCT |
| | CGTGAAAGTGATGGGCGGCCACAAGCCCGAGAACATCGTGA |
| | TCGAAATGGCCAGAGAGAACCAGACCACCCAGAAGGGACA |
| | GAAGAACAGCCGCGAGAGAATGAAGCGGATCGAAGAGGGC |
| | ATCAAAGAGCTGGGCAGCCAGATCCTGAAAGAACACCCCGT |
| | GGAAAACACCCAGCTGCAGAACGAGAAGCTGTACCTGTACT |
| | ACCTGCAGAATGGGCGGGATATGTACGTGGACCAGGAACTG |
| | GACATCAACCGGCTGTCCGACTACGATGTGGACCATATCGTG |
| | CCTCAGAGCTTTCTGAAGGACGACTCCATCGACAACAAGGT |
| | GCTGACCAGAAGCGACAAGAACCGGGGCAAGAGCGACAAC |
| | GTGCCCTCCGAAGAGGTCGTGAAGAAGATGAAGAACTACTG |
| | GCGGCAGCTGCTGAACGCCAAGCTGATTACCCAGAGAAAGT |
| | TCGACAATCTGACCAAGGCCGAGAGAGGCGGCCTGAGCGAA |
| | CTGGATAAGGCCGGCTTCATCAAGAGACAGCTGGTGGAAAC |
| | CCGGCAGATCACAAAGCACGTGGCACAGATCCTGGACTCCC |
| | GGATGAACACTAAGTACGACGAGAATGACAAGCTGATCCGG |
| | GAAGTGAAAGTGATCACCCTGAAGTCCAAGCTGGTGTCCGA |
| | TTTCCGGAAGGATTTCCAGTTTTACAAAGTGCGCGAGATCAA |
| | CAACTACCACCACGCCCACGACGCCTACCTGAACGCCGTCGT |
| | GGGAACCGCCCTGATCAAAAAGTACCCTAAGCTGGAAAGCG |
| | AGTTCGTGTACGGCGACTACAAGGTGTACGACGTGCGGAAG |
| | ATGATCGCCAAGAGCGAGCAGGAAATCGGCAAGGCTACCGC |
| | CAAGTACTTCTTCTACAGCAACATCATGAACTTTTTCAAGAC |
| | CGAGATTACCCTGGCCAACGGCGAGATCCGGAAGCGGCCTC |
| | TGATCGAGACAAACGGCGAAACCGGGGAGATCGTGTGGGAT |
| | AAGGGCCGGGATTTTGCCACCGTGCGGAAAGTGCTGAGCAT |
| | GCCCCAAGTGAATATCGTGAAAAAGACCGAGGTGCAGACAG |
| | GCGGCTTCAGCAAAGAGTCTATCCTGCCCAAGGGCAACAGC |
| | GATAAGCTGATCGCCAGAAAGAAGGACTGGGACCCTAAGAA |
| | GTACGGCGGCTTCAACAGCCCCACCGTGGCCTATTCTGTGCT |
| | GGTGGTGGCCAAAGTGGAAAAGGGCAAGTCCAAGAAACTGA |
| | AGAGTGTGAAAGAGCTGCTGGGGATCACCATCATGGAAAGA |
| | AGCAGCTTCGAGAAGAATCCCATCGACTTTCTGGAAGCCAA |
| | GGGCTACAAAGAAGTGAAAAAGGACCTGATCATCAAGCTGC |
| | CTAAGTACTCCCTGTTCGAGCTGGAAAACGGCCGGAAGAGA |
| | ATGCTGGCCTCTGCCGGCGTGCTGCAGAAGGGAAACGAACT |
| | GGCCCTGCCCTCCAAATATGTGAACTTCCTGTACCTGGCCAG |
| | CCACTATGAGAAGCTGAAGGGCTCCCCCGAGGATAATGAGC |
| | AGAAACAGCTGTTTGTGGAACAGCACAAGCACTACCTGGAC |
| | GAGATCATCGAGCAGATCAGCGAGTTCTCCAAGAGAGTGAT |
| | CCTGGCCGACGCTAATCTGGACAAAGTGCTGTCCGCCTACAA |
| | CAAGCACCGGGATAAGCCCATCAGAGAGCAGGCCGAGAATA |
| | TCATCCACCTGTTTACCCTGACCAATCTGGGAGCCCCTGCCG |
| | CCTTCAAGTACTTTGACACCACCATCAACCGGAAGCAATACA |
| | ACACGACCAAAGAGGTGCTGGACGCCACCCTGATCCGTCAG |
| | AGCATCACCGGCCTGTACGAGACACGGATCGACCTGTCTCA |
| | GCTGGGAGGTGACTCTGGCGGCTCAAAAAGAACCGCCGACG |
| | GCAGCGAATTCGAGCCCAAGAAGAAGAGGAAAGTCTAA |
| | (SEQ ID NO: 7) |
| HBBG- | 5′-UUCUCCACAGGAGUCAGGUG-3′ (SEQ ID NO: 8) |
| Makassar | |
| sgRNA | |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition for targeted delivery of gene editing agents to a target hematopoietic stem cell (HSC), the composition comprising a first delivery vehicle comprising an mRNA molecule encoding a Cas9 base editor and a second delivery vehicle comprising a short guide RNA (sgRNA), wherein each of the first and second delivery vehicle comprises a CD117 targeting moiety.

2. The composition of claim 1, wherein each of the first and second delivery vehicles comprises a lipid nanoparticle (LNP).

3. The composition of claim 2, wherein the gene editing agents are encapsulated within the LNPs.

4. The composition of claim 3, wherein the mRNA molecule encoding a Cas9 base editor encodes an adenine base editor (ABE) or a cytidine base editor (CBE).

5. The composition of claim 4, wherein the mRNA encoding the ABE is transcribed from a nucleic acid molecule comprising SEQ ID NO:7.

6. The composition of claim 5, wherein the mRNA molecule is an isolated nucleoside-modified mRNA molecule.

7. The composition of claim 6, wherein the at least one isolated nucleoside-modified RNA comprises at least one selected from the group consisting of pseudouridine and 1-methyl-pseudouridine.

8. The composition of claim 3, wherein the targeting moiety of each of the first and second delivery vehicle comprises a CD117 antibody.

9. The composition of claim 1, wherein the second delivery vehicle is in excess of the first delivery vehicle.

10. A method of treating a disease or disorder in a subject in need thereof, the method comprising administering a composition of claim 1 to the subject.

11. The method of claim 10, wherein the disease or disorder is selected from the group consisting of blood monogenic disorders, genetic defects, bone marrow genetic defects, cancers, platelet disorders, immunodeficiencies, metabolic disease and autoimmune diseases.

12. The method of claim 11, wherein the disease or disorder is a sickle cell disease.

13. The method of claim 12, wherein the sickle cell disease is sickle cell anemia.

14. The method of claim 13, wherein the sgRNA targets a hemoglobin subunit beta (HBB) gene.

15. The method of claim 14, wherein the sgRNA comprises the nucleotide sequence of SEQ ID NO:8.

16. The method of claim 10, wherein the composition is administered by a delivery route selected from the group consisting of intravenous (IV), intraosseous infusion (IO), intraperitoneal (IP), intradermal, subcutaneous, inhalation, intranasal, and intramuscular.
